(19) United States
(12) Patent Application Publication
(10) Pub. No.: US 2022/0227776 A1
(43) Pub. Date: Jul. 21, 2022

(54) PRODRUGS OF 1'-SUBSTITUTED CARBA-NUCLEOSIDE ANALOGUES FOR ANTIVIRAL TREATMENT

(71) Applicant: Copycat Sciences Inc., Brighton, MA (US)

(72) Inventors: Victoria Yan, Hillsborough, CA (US); Matthew Yan, Hillsborough, CA (US)

(73) Assignee: Copycat Sciences Inc., Brighton, MA (US)

(21) Appl. No.: 17/712,635

(22) Filed: Apr. 4, 2022

Related U.S. Application Data
(63) Continuation-in-part of application No. PCT/US2020/059724, filed on Nov. 9, 2020.

(51) Int. Cl. C07D 487/04; C07F 9/117

(52) U.S. Cl. CPC C07D 487/04; C07F 9/117

(57) ABSTRACT

The present invention provides novel compounds and pharmaceutically acceptable salts or esters thereof. For example, the compound has the structure of Formula V. Also provided is a pharmaceutical composition comprising the compound or a pharmaceutically acceptable salt or ester thereof and a pharmaceutically acceptable carrier. Further provided a method for inhibiting a polymerase of Severe acute respiratory syndrome coronavirus 2 (SARS-CoV-2) (SARS-CoV-2 polymerase) or treating viral infection in a subject in need thereof, comprising administering an effective amount of the pharmaceutical composition to the subject, for example, orally.

### CROSS-REFERENCE TO RELATED APPLICATION

This application is a continuation-in-part of International Application No. PCT/US2020/059724, filed Nov. 9, 2020, the contents of which are incorporated herein by reference.

### FIELD OF THE INVENTION

The present disclosure relates generally to compounds with antiviral activity, more particularly nucleosides active against Coronaviridae infections and most particularly to inhibitors of SARS-CoV-2 RNA-dependent RNA polymerase.

### BACKGROUND OF THE INVENTION

Viruses comprising the Coronaviridae family comprise two subfamilies, including coronaviruses and toroviruses (Payne, Viruses, 2017, 149-158). The coronavirus subfamily comprises at least four distinguishable genera, including Alpha coronavirus, Beta coronavirus, Gamma coronavirus, and Delta coronavirus. The torovirus subfamily comprises at least two distinguishable genera, including torovirus and bafinivirus. Toroviruses primarily infect vertebrates such as cattle, pig, and horses and can also infect humans (Hoet, A., Encyc. Virol., 2008, 151-57). The main disease associated with toroviruses is gastroenteritis (Wilhelmi, I., et al. Clin. Microb. Infect. 2003, 9:4; 47-262). In humans, coronaviruses primarily cause respiratory tract infections that can range from mild to lethal severity. They are responsible for important human diseases such as the common cold, severe acute respiratory syndrome (SARS), Middle East respiratory syndrome (MERS) (Fehr, A. R., and Perlman, S. Coronaviruses. 2015, 1282, 1-23) and coronavirus disease 2019 (COVID-19) (Wiersinga, W. J., et al. J. Am. Med. Assoc. 2020, 324:8; 782-793). There are currently no vaccines or antiviral drugs that have been successfully developed to prevent or treat human coronavirus infections. Therefore, there is a need to develop effective treatments for Coronaviridae virus infections.

Severe acute respiratory syndrome coronavirus 2 (SARS-CoV-2) is the cause of the COVID-19 pandemic (Wiersinga, W. J., et al. J. Am. Med. Assoc. 2020, 324; 8, 782-793), so a significant focus of current antiviral research has been directed toward the identification and development of therapeutics and methods of treatment of COVID-19 in humans (Wang, M., et al. Cell Res. 2020, 30, 269-271; Cao, B., et al. N. Engl. J. Med. 2020, 382, 1787-1799). A number of COVID-19 treatments that have been or are currently being investigated are reviewed by Lythgoe and Middleton in Trends in Pharmacological Sciences, 41:6; 363-382 (2020).

RNA-dependent RNA polymerase (RdRp) is one of more well-studied targets for development of novel COVID-19 therapeutic agents (Gordon, C. J., et al. J. Biol. Chem. 2020, 295:20; 6785-6797; Tchesnokov, E. P., et al. J. Biol. Chem. 2020; Yin, W., et al. Science 2020, 368:6498, 1499-1504). The SARS-CoV-2 RdRp is a target for inhibitors in early and late clinical trials (Beigel, J. B., et. al. N. Engl. J. Med., 2020; NEJMoa2007764; NCT04405739, NCT03891420). This enzyme has been extensively characterized at the biochemical and structural levels (Gordon. C. J., et al. J. Biol. Chem. 2020, 295:20; 6785-6797; Tchesnokov, E. P., et al. J. Biol. Chem. 2020; Yin, W., et al. Science 368:6498, 1499-1504; Pruijssers, A. J., et al. Cell Rep. 32:3;107940), with screening assays for identifying selective inhibitors (NCATS OpenData Portal).

Currently, there are two therapies that have demonstrated some efficacy in treating humans with severe COVID-19. These include the FDA-approved antiviral nucleotide analogue prodrug, remdesivir (GS-5734, Veklury®), and the steroid dexamethasone. Remdesivir has shown efficacy in reducing time for hospitalization (Beigel, J. B., et. al. N. Engl. J. Med., 2020; NEJMoa2007764) and dexamethasone has shown efficacy for reducing mortality (RC Group et al. N. Engl. J. Med., 2020; NEJMMoa2021436). While remdesivir has demonstrated the ability to reduce viral RNA levels in preclinical models of SARS-CoV-2 (Williamson, B. N., et al. Nature, 2020, 585:7824, 273-276), no published data exist on its ability to reduce viral RNA levels in humans treated for COVID-19. A significant shortcoming with remdesivir is its requirement for intravenous administration, which impedes therapeutic or prophylactic treatment in the outpatient setting. Another significant shortcoming with remdesivir is that its moderate efficacy in humans with severe COVID-19 cannot be improved by further increasing the dose administered due to dose-limiting toxicities to the liver and kidneys (FDA, Fact Sheet for Health Care Providers for Emergency Use Authorization (EUA) of Veklury® (remdesivir)). Dexamethasone is a corticosteroid that reduces inflammation associated with severe manifestations of COVID-19 (Huang, C., et al. Lancet, 2020, 395; 10223, 497-506; Moore, J. B., and June, C. H., Science, 2020, 368; 6490, 473-473) and does not directly intervene with the SARS-CoV-2 replication machinery. Therefore, it is unclear whether humans with mild forms of COVID-19 that lack inflammatory organ injury would benefit from treatment with dexamethasone. Formal studies on the combination of remdesivir and dexamethasone for the treatment of humans with mild or severe COVID-19 have not been published. Currently, the only reported case of a human with severe COVID-19 administered the combination treatment has been the President of the United States, Donald J. Trump. Other patent applications disclosing the use of nucleoside analogues to treat coronaviridae virus infections include US2017/0071964, US2015/0291596, US2004/0259934, US2019/0255085, KR2145197, CN111454270, CN111233929, WO2019/173602, WO2019/027501, WO2019/053696, WO2019/027501, WO2018/169946, WO 2016/123318, WO2003/018030. However, most of the compounds and methods described in these the patent applications are have not demonstrated efficacy against COVID-19, with few currently being investigated in clinical trials. Given the modest efficacy and limited scope of humans who would benefit from remdesivir and or dexamethasone, the development of antiviral agents with improved pharmacokinetic properties, improved oral bioavailability, greater efficacy, fewer undesirable side effects, and extended half-life in vivo (Yan, V. C. and Muller, F. L., 2020, ACS Med. Chem. Lett., 11; 7, 1361-1366) is urgently needed.

Certain ribosides of the nucleobases pyrrolo[1,2-f][1,2,4]triazine have been disclosed in WO 2009132135; Bioorganic Medicinal Chemistry Letters 2012, 22(8), 2705-270; Nature 2016, 531(7594), 381-385; Journal of Medicinal Chemistry, 2017 60(5), 1648-1661; US/20170071964; Veterinary Microbiology, 2018 219, 226-233; Journal of Feline Medicine Surgery, 2019, 21(4), 271-281; US2019/0255085; Journal of Veterinary Internal Medicine, 2020 34(4), 1587-1593. However, the compounds disclosed in this patent application have neither been previously been reported in these publications nor have they been previously disclosed as useful for the treatment of COVID-19. Clarke, M. O., US2019/0255085 and Perron, M. J., WO2018/169946 discloses ribosides of pyrrolo[1,2-f][1,2,4]triazine nucleobases with antiviral, anti-MERS-CoV, anti-SARS-CoV, and anti-feline coronavirus (FCoV) activity.

GS-443902 (also known as GS-441524 triphosphate) is an adenosine triphosphate (ATP) analogue that is specific for RNA viruses that express RNA-dependent RNA polymerase (RdRp) because it is a potent viral RdRp inhibitor. GS-443902 is a polyanion that has poor cell-permeability and is unstable in plasma; as a result, there is interest in developing nucleoside and nucleotide analogue precursor prodrugs of GS-443902. Monophosphate and phosphoramidate-containing prodrugs have been developed for intracellular delivery of GS-443902. An example of an FDA-approved prodrug of GS-443902 is remdesivir (GS-5734) as discussed above. There remains a need for orally bioavailable nucleoside prodrugs that, in contrast to most clinically advanced prodrugs of GS-443902, are not monophosphate or phosphoramidate prodrugs.

### SUMMARY OF THE INVENTION

The present invention provides novel compounds, or pharmaceutically acceptable salts or esters thereof, and their uses as prodrugs of GS-443902 in antiviral treatments. The invention is based on the surprising discovery by the inventors that CAT002 was efficiently metabolized to GS-441524 when administered orally to human subjects. GS-441524 is the core nucleoside of CAT002 and does not have the 5′ amino acid ester in CAT002.

A first compound or a pharmaceutically acceptable salt or ester thereof is provided. The first compound has the structure of Formula I:

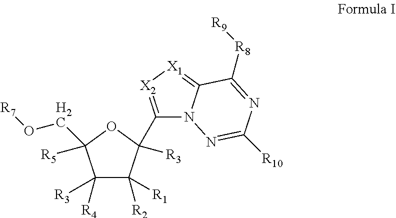

wherein:

R¹, R², R², R³, R⁴, and R⁵ are each independently H, OR^{a}, N(R^{a})₂, N₃, CN, NO₂, S(O)ₙR^{a}, halogen, (C₁-C₈)alkyl, (C₄-C₈)carbocyclalkyl, (C₁-C₈) substituted alkyl, (C₂-C₈) alkenyl, (C₂-C₈)substituted alkenyl, (C₂-C₈)alkynyl, (C₂-C₈)substituted alkynyl or aryl(C₂-C₈)alkyl; or any two R¹, R², R³, R⁴, or R⁵ on adjacent carbon atoms when taken together are —O(CO)O— or when taken together with the ring carbon atoms to which they are attached form a double bond;

R⁶ is OR^{a}, N(R^{a})₂, N₃, CN, NO₂, S(O)ₙR^{a}, —C(═O)OR¹¹, —C(═O)NR¹¹R¹², —C(═O)SR¹¹, ═S(O)R¹¹, ═S(O)₂R¹¹, —S(O)(OR¹¹), —S(O)₂(OR¹¹), —SO₂NR¹¹R¹², halogen, (C₁-C₈)alkyl, (C₁-C₈)carbocyclylalkyl, (C₁-C₈)substituted alkyl, (C₂-C₈)alkenyl, (C₂-C₈)substituted alkenyl, (C₂-C₈)alkynyl, (C₂-C₈)substituted alkynyl, or (C₆-C₂₀)aryl(C₁-C₈)alkyl;

R⁷ and R⁹ are each independently selected from a group consisting of:

a) H, —C(═O)R¹¹, —C(═O)NR¹¹R¹², C(═O)SR¹¹, ═S(O)R¹¹, ═S(O)₂R¹¹, —S(O)(OR¹¹), —S(O)₂(OR¹¹), —SO₂NR¹¹R¹²; and

b)

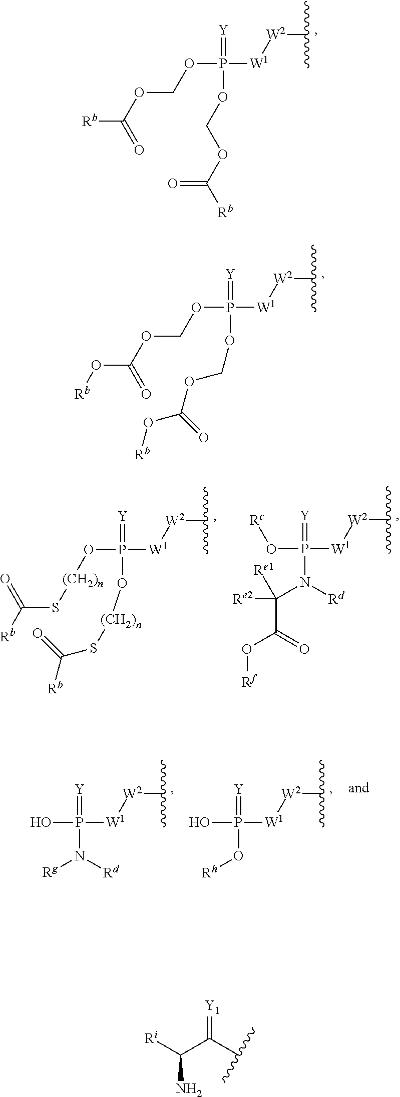

wherein:

Y is O, S, NR, ⁺N(O)(R), N(OR), ⁺N(O)(OR), or N—NR₂;

Y¹ is O or S;

W¹ and W² are each independently O, S, NR, N(OR), CR₂, or C(X⁴)₂;

R^{b} is (C₁-C₈)alkyl, (C₁-C₈)carbocyclylalkyl, (C₁-C₈)substituted alkyl, or (C₆-C₂₀)aryl(C₁-C₈)alkyl;

R^{c} is phenyl, 1-naphthyl, 2-naphthyl,

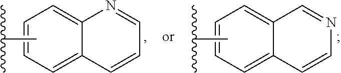

R^{d} is H or CH₃;

R^{e1} and R^{e2} are each independently H, (C₁-C₆)alkyl, benzyl, or halogen;

R^{f} is H, (C₁-C₈)alkyl, benzyl, (C₃-C₆)cycloalkyl, or —CH₂—(C₃-C₆)cycloalkyl;

R^{g} is H, CH₃, (C₁-C₁₂)alkyl,(C₁-C₈)carbocyclylalkyl, (C₁-C₈)substituted alkyl or (C₆-C₂₀)aryl(C₁-C₈)alkyl; alkyl;
R^{h} is

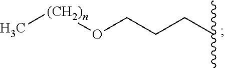

R⁸ is NH, or NR;

R¹⁰ is H, halogen, NR¹¹R¹², N(R¹¹)OR¹¹, N R¹¹N R¹¹1R¹², N₃, NO, NO₂, CHO, CN, —CH(═NR¹¹), —CH═NHNR¹¹, —CH═N(OR¹¹), —CH(OR¹¹)2, —C(═O)NR¹¹R¹², —C(═S)NR¹¹R¹², —C(═OR)OR¹¹, R¹¹, OR¹¹, or SR″;

R^{a} is H, (C₁-C₈)alkyl, (C₂-C₈) alkenyl, (C₂-C₈)alkynyl, aryl(C₁-C₈)alkyl, (C₄-C₈)carbocyclylalkyl, —C(═O)R¹¹, —C(═O)OR¹¹, —C(═O)NR¹¹R¹², —C(═O)SR¹¹, —S(O)R¹¹, —S(O)₂R¹¹, —S(O)(OR¹¹), —S(O)₂(OR¹¹), or SO₂NR¹¹R¹²;

R¹¹ and R¹² are each independently H, (C₁-C₈)alkyl, (C₂-C₈)alkenyl, or (C₁-C₈)carbocyclylalkyl;

n is 0, 1, or 2;

X¹ and X² are each independently C, C—R¹³, or N; and

R¹³ is H or halogen.

A second compound or a pharmaceutically acceptable salt or ester thereof is provided. The second compound has the structure of Formula II:

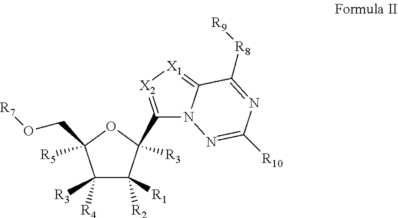

wherein:

R¹, R², R³, R⁴, and R⁵ are each independently H, OR^{a}, N(R^{a})₂, N₃, CN, NO₂, S(O)ₙR^{a}, halogen, (C₁-C₈)alkyl, (C₄-C₈)carbocyclalkyl, (C₁-C₈) substituted alkyl, (C₂-C₈) alkenyl, (C₂-C₈)substituted alkenyl, (C₂-C₈)alkynyl, (C₂-C₈)substituted alkynyl or aryl(C₂-C₈)alkyl; or any two R¹, R², R³, R⁴, or R⁵ on adjacent carbon atoms when taken together are —O(CO)O— or when taken together with the ring carbon atoms to which they are attached form a double bond;

R⁶ is OR^{a}, N(R^{a})₂, N₃, CN, NO₂, S(O)ₙR^{a}, —C(═O)OR¹¹, —C(═O)NR¹¹R¹², —C(═O)SR¹¹, ═S(O)R¹¹, ═S(O)₂R¹¹, —S(O)(OR¹¹), —S(O)₂(OR¹¹), —SO₂NR¹¹R¹², halogen, (C₁-C₈)alkyl, (C₁-C₈)carbocyclylalkyl, (C₁-C₈)substituted alkyl, (C₂-C₈)alkenyl, (C₂-C₈)substituted alkenyl, (C₂-C₈)alkynyl, (C₂-C₈)substituted alkynyl, or (C₆-C₂₀)aryl(C₁-C₈)alkyl;

R⁷ and R⁹ are each independently selected from a group consisting of:

a) H, —C(═O)R¹¹, —C(═O)NR¹¹R¹², C(═O)SR¹¹, ═S(O)R¹¹, ═S(O)₂R¹¹, —S(O)(OR¹¹), —S(O)₂(OR¹¹), —SO₂NR¹¹R¹²;
b)

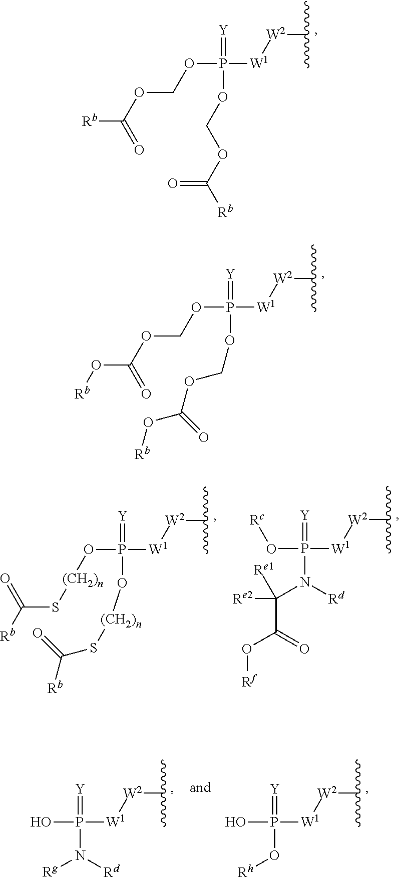

and

c.)

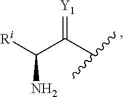

wherein:
Y is O;
Y¹ is O;
W¹ and W² are each independently O, S, NR, N(OR), CR₂, or C(X⁴)₂;
R^{b} is (C₁-C₈)alkyl, (C₁-C₈)carbocyclylalkyl, (C₁-C₈)substituted alkyl, or (C₆-C₂₀)aryl(C₁-C₈)alkyl;
R^{c} is phenyl, 1-naphthyl, 2-naphthyl,

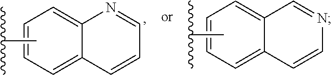

R^{d} is H or CH₃;
R^{e1} and R^{e2} are each independently H, (C₁-C₆)alkyl, benzyl, or halogen;
R^{f} is H, (C₁-C₈)alkyl, benzyl, (C₃-C₆)cycloalkyl, or —CH₂—(C₃-C₆)cycloalkyl;
R^{g} is H, CH₃, (C₁-C₁₂)alkyl,(C₁-C₈)carbocyclylalkyl, (C₁-C₈)substituted alkyl or (C₆-C₂₀)aryl(C₁-C₈)alkyl; alkyl;
R^{h} is

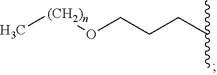

Rⁱ is (C₁-C₈)alkyl, (C₁-C₈)substituted alkyl, (C₁-C₈)carbocyclylalkyl, or (C₆-C₂₀)aryl(C₁-C₈)alkyl;

R⁸ is NH, or NR;

R¹⁰ is H, halogen, NR¹¹R¹², N(R¹¹)OR¹¹, N R¹¹N R¹¹1R¹², N₃, NO, NO₂, CHO, CN, —CH(═NR¹¹), —CH═NHNR¹¹, —CH═N(OR¹¹), —CH(OR¹¹)2, —C(═O)NR¹¹R¹², —C(═S)NR¹¹R¹², —C(═OR)OR¹¹, R¹¹, OR¹¹, or SR¹¹;

R^{a} is H, (C₁-C₈)alkyl, (C₂-C₈) alkenyl, (C₂-C₈)alkynyl, aryl(C₁-C₈)alkyl, (C₄-C₈)carbocyclylalkyl, —C═O)R¹¹, —C═O)OR¹¹, —C(═C)NR¹¹R¹², —C(═O)SR¹¹, —S(O)R¹¹, —S(O)2R¹¹, —S(O)(OR¹¹), —S(O)₂(OR¹¹), or SO₂NR¹¹R¹²;

R¹¹ and R¹² are each independently H, (C₁-C₈)alkyl, (C₂-C₈)alkenyl, or (C₁-C₈)carbocyclylalkyl;

n is 0, 1, or 2;

X¹ is C, C—R¹³, or N;

X² is C—R¹³; and

R¹³ is H or halogen.

With respect to a first compound or a pharmaceutically acceptable salt or ester thereof according to the present invention, the compound may have the structure of Formula III:

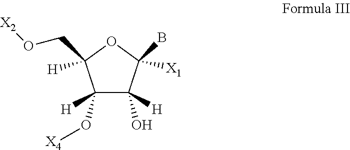

wherein X1 is defined in table 1, X2 is defined in table 2, X4 is defined in table 4, B1 is defined in table 5 and X3 is defined in table 3. X2 may be selected from the group consisting of X2a, X2b, X2c and X2d as defined in table 2.

A third compound or a pharmaceutically acceptable salt or ester thereof is provided. The third compound may have the structure of Formula V:

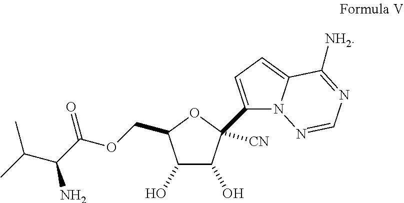 [SMILES: CC(C)[C@H](N)C(=O)OC[C@H]1O[C@@](C#N)(c2ccc3c(N)ncnn23)[C@H](O)[C@@H]1O]

With respect to a first compound or a pharmaceutically acceptable salt or ester thereof according to the present invention, the compound may be

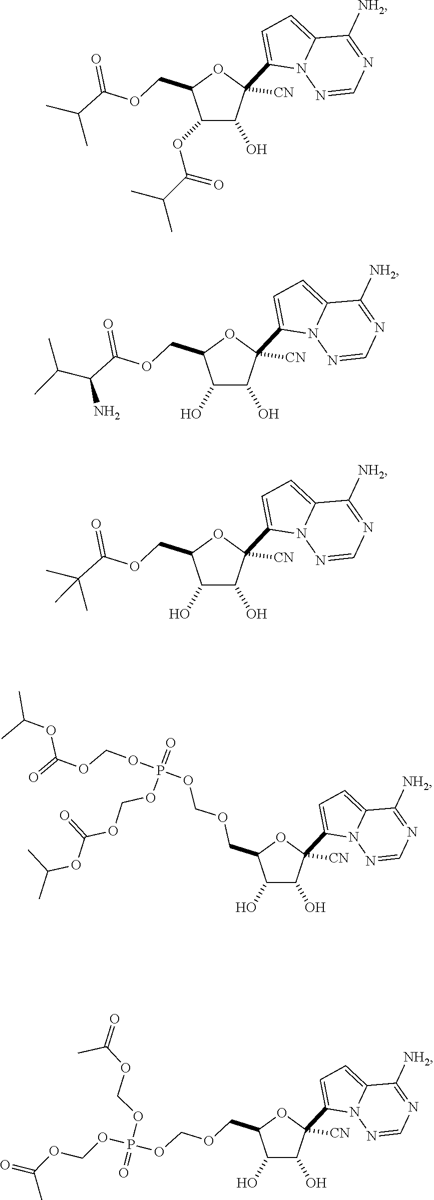

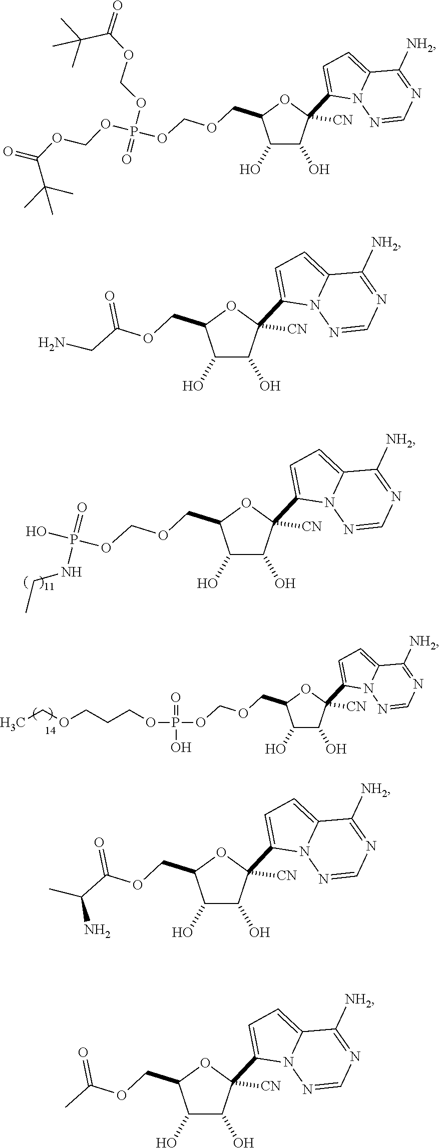

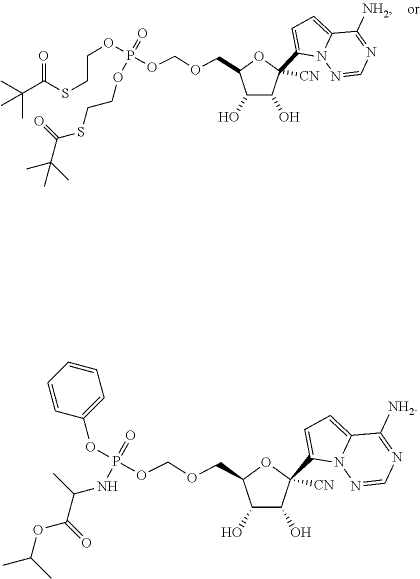

A pharmaceutically acceptable salt of a compound according to the present invention may be an acid or base salt. The pharmaceutically acceptable salt of the compound may be of sufficient purity and quality for use in formulation of a pharmaceutical composition or medicament. The pharmaceutically acceptable salt of the compound may be tolerated and sufficiently non-toxic to be used in a pharmaceutical preparation. Suitable pharmaceutically acceptable salts include acid addition salts, which may, for example, be formed by reacting the compound with a suitable pharmaceutically acceptable acid such as hydrochloric acid, hydrobromic acid, sulfuric acid, fumaric acid, maleic acid, succinic acid, acetic acid, benzoic acid, citric acid, tartaric acid, carbonic acid or phosphoric acid. In one embodiment, the pharmaceutical acceptable salt is a halide salt, for example, a chloride salt.

A pharmaceutically acceptable ester of a compound according to the present invention may be esters formed from carboxy, sulfonyloxy, and phosphonoxy groups present in the compound, e.g., -alkyl esters. In one embodiment, the pharmaceutically acceptable ester is an amino acid ester.

Where two acidic groups are present, a pharmaceutically acceptable salt or ester may be a mono-acid-mono-salt or ester or a di-salt or ester. Similarly, where more than two acidic groups are present, some or all of such acidic groups may be in a salt or ester form.

A pharmaceutical composition is provided for inhibiting a polymerase of Severe acute respiratory syndrome coronavirus 2 (SARS-CoV-2) (SARS-CoV-2 polymerase). The pharmaceutical composition comprises an effective amount of a compound or a pharmaceutically acceptable salt or ester thereof according to the present invention and a pharmaceutically acceptable carrier. The compound may have the structure of Formula I, Formula II, Formula III, Formula IV or Formula V. In one embodiment, the pharmaceutical composition comprises the compound having the structure of Formula V. The pharmaceutical composition may further comprise an additional therapeutic agent. The additional therapeutic agent may be selected from the group consisting of interferons, monoclonal antibodies, corticosteroids, 3CL protease inhibitors, SSRIs, IL-6 inhibitors, JAK inhibitors, antihyperuricemic agents, non-nucleoside inhibitors of SARS-CoV-2, and other drugs for treating SARS-CoV-2.

A pharmaceutical composition is provided for treating a viral infection caused by a virus of the Coronaviridae family. The pharmaceutical composition comprises an effective amount of a compound or a pharmaceutically acceptable salt or ester thereof according to the present invention and a pharmaceutically acceptable carrier. The compound may have the structure of Formula I, Formula II, Formula III, Formula IV or Formula V. In one embodiment, the pharmaceutical composition comprises the compound having the structure of Formula V. The pharmaceutical composition may further comprise an additional therapeutic agent. The additional therapeutic agent may be selected from the group consisting of interferons, monoclonal antibodies, corticosteroids, 3CL protease inhibitors, SSRIs, IL-6 inhibitors, JAK inhibitors, antihyperuricemic agents, non-nucleoside inhibitors of Severe acute respiratory syndrome coronavirus 2 (SARS-CoV-2), and other drugs for treating SARS-CoV-2.

A pharmaceutically acceptable carrier may be selected from the group consisting of a pharmaceutically acceptable solvent, a suspending agent, a vehicle for delivering the compound or a pharmaceutically acceptable salt or ester thereof according to the present invention, and combinations thereof. The pharmaceutically acceptable carrier may be an ingredient, excipient or component suitable for use with humans and/or animals without undue adverse side effects, for example, toxicity, irritation and allergic response, commensurate with a reasonable balance of associated benefits and risks.

A method of inhibiting a polymerase of Severe acute respiratory syndrome coronavirus 2 (SARS-CoV-2) (SARS-CoV-2 polymerase) in a subject in need thereof is provided. The method comprises administering to the subject an effective amount of the pharmaceutical composition of the present invention. The pharmaceutical composition may be administered to the subject orally. The effective amount of the pharmaceutical composition may be selected to provide a maximum plasma concentration (Cmax) of the compound of Formula V a pharmaceutically acceptable salt or ester thereof in the subject in the range of 3-10 μM. The effective amount of the pharmaceutical composition may be selected to provide a maximum plasma concentration (Cmax) of a metabolite of the compound of Formula V, or a pharmaceutically acceptable salt or ester thereof, in the subject in the range of 3-10 μM. The metabolite may be GS-441524. The pharmaceutical composition may be administered to the subject in an amount effective for inhibiting at least 10%, 20%, 30%, 40%, 50%, 60%, 70%, 80%, 90%, 95% or 99% of the activity of the SARS-CoV-2 polymerase.

A method of treating a viral infection in a subject in need thereof is provided. The viral infection is caused by a virus of the Coronaviridae family. The method comprises administering to the subject an effective amount of the pharmaceutical composition of the present invention. The pharmaceutical composition may be administered to the subject orally. The effective amount of the pharmaceutical composition may be selected to provide a maximum plasma concentration (Cmax) of the compound of Formula V a pharmaceutically acceptable salt or ester thereof in the subject in the range of 3-10 μM. The effective amount of the pharmaceutical composition may be selected to provide a maximum plasma concentration (Cmax) of a metabolite of the compound of Formula V, or a pharmaceutically acceptable salt or ester thereof, in the subject in the range of 3-10 μM. The metabolite may be GS-441524. The virus may be selected from the group consisting of dengue virus, yellow fever virus, West Nile virus, Japanese encephalitis virus, St. Louis encephalitis virus, Omsk hemorrhagic fever, Ebola virus, bovine viral diarrhea virus, Zika virus, Marburg virus, Hepatitis C virus, human coronavirus 229E, human coronavirus OC43, Middle East Respiratory Syndrome virus, Severe Acute Respiratory Syndrome virus, and Severe Acute Respiratory Syndrome coronavirus 2 (SARS-CoV-2). In one embodiment, the virus is SARS-CoV-2. The method may further comprise administering to the subject an additional therapeutic agent selected from the group consisting of interferons, monoclonal antibodies, corticosteroids, 3CL protease inhibitors, SSRIs, IL-6 inhibitors, JAK inhibitors, antihyperuricemic agents, non-nucleoside inhibitors of SARS-CoV-2, and other drugs for treating SARS-CoV-2. The pharmaceutical composition may be administered to the subject in an amount effective for reducing at least 10%, 20%, 30%, 40%, 50%, 60%, 70%, 80%, 90%, 95% or 99% of the viral infection. The effectiveness of treatment may be determined by conventional techniques known in the art for the viral infection, for example, by quantification of viral load in a sample of plasma or bronchial alveolar lavage fluid from the subject, or negative PCR or rapid antigen test.

A method for preparing the pharmaceutical composition for inhibiting a polymerase of Severe acute respiratory syndrome coronavirus 2 (SARS-CoV-2) (SARS-CoV-2 polymerase) is provided. The preparation method comprises mixing a compound or a pharmaceutically acceptable salt or ester thereof according to the present invention with the pharmaceutically acceptable carrier. The compound may have the Formula I, Formula II, Formula III, Formula IV or Formula V. For example, the pharmaceutical composition comprises the compound having the structure of Formula V.

A method for preparing the pharmaceutical composition for treating a viral infection caused by a virus of the Coronaviridae family is provided. The preparation method comprises mixing a compound or a pharmaceutically acceptable salt or ester thereof according to the present invention with the pharmaceutically acceptable carrier. The compound may have the Formula I, Formula II, Formula III, Formula IV or Formula V. For example, the pharmaceutical composition comprises the compound having the structure of Formula V.

The subject may be a mammal, for example, a human. The subject may be a healthy individual or a patient. The subject may be in need of a compound or a pharmaceutically acceptable salt or ester thereof according to the present invention. The subject may be in need of inhibition of a polymerase of Severe acute respiratory syndrome coronavirus 2 (SARS-CoV-2) (SARS-CoV-2 polymerase). The subject may be in need of treatment of viral infection.

The term “an effective amount” refers to an amount of a pharmaceutical composition comprising a compound or a pharmaceutically acceptable salt or ester thereof according to the present invention required to achieve a stated goal (e.g., inhibiting a polymerase of Severe acute respiratory syndrome coronavirus 2 (SARS-CoV-2) (SARS-CoV-2 polymerase) or treating viral infection in a subject in need thereof). The effective amount of the pharmaceutical composition may be selected to achieve a maximum plasma concentration of the compound in the subject in the range of about 3-10 μM, with one or multiple doses of the composition. The effective amount of the pharmaceutical composition may be selected to provide a maximum plasma concentration (Cmax) of a metabolite of the compound of Formula V, or a pharmaceutically acceptable salt or ester thereof, in the subject in the range of 3-10 μM. The metabolite may be GS-441524. The effective amount of the pharmaceutical composition comprising the compound or the pharmaceutically acceptable salt or ester thereof may vary depending upon the stated goals, the physical characteristics of the subject, the nature and severity of the viral infection, the existence of related or unrelated medical conditions, the nature of the compound or the pharmaceutically acceptable salt or ester thereof, the composition comprising the compound or the pharmaceutically acceptable salt or ester thereof, the means of administering the composition to the subject, and the administration route. A specific dose for a given subject may generally be set by the judgment of a physician. The pharmaceutical composition may be administered to the subject in one or multiple doses. Each dose may be at about 100-1,000 mg.

### BRIEF DESCRIPTION OF THE DRAWINGS

FIG. 1 shows plasma pharmacokinetics of GS-441524 following oral administration of CAT002 in Sprague-Dawley rats.

FIG. 2 shows plasma pharmacokinetics of GS-441524 following oral administration of CAT002 in cynomolgus macaques.

FIG. 3 shows plasma pharmacokinetics of GS-441524 following oral administration of CAT002 in humans.

### DETAILED DESCRIPTION OF THE INVENTION

### I. COMPOUNDS OF THE PRESENT INVENTION

Reference will now be made in detail to certain embodiments of the invention, examples of which are illustrated in the accompanying description, structures and formulas. While the invention will be described in conjunction with the enumerated embodiments, it will be understood that they are not intended to limit the invention to those embodiments. On the contrary, the invention is intended to cover all alternatives, modifications, and equivalents which may be included within the scope of the present invention.

This instant invention provides compounds that inhibit viruses of the Coronaviridae family. This invention also comprises compounds that inhibit viral nucleic acid polymerases, particularly SARS-CoV-2 RNA-dependent RNA polymerase (RdRp), rather than cellular nucleic acid polymerases. Therefore, the compounds of the instant invention are useful for treating Coronaviridae infections in humans and other animals.

In one aspect, this invention provides a compound of Formula I:

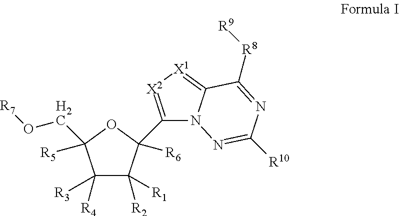

or a pharmaceutically acceptable salt or ester thereof;
wherein:

each R¹, R², R³, R⁴, or R⁵ is independently H, OR^{a}, N(R^{a})₂, N₃, CN, NO₂, S(O)ₙR^{a}, halogen, (C₁-C₈)alkyl, (C₄-C₈)carbocyclalkyl, (C₁-C₈) substituted alkyl, (C₂-C₈) alkenyl, (C₂-C₈)substituted alkenyl, (C₂-C₈)alkynyl, (C₂-C₈)substituted alkynyl or aryl(C₂-C₈)alkyl;
or any two R¹, R², R³, R⁴, or R⁵ on adjacent carbon atoms when taken together are —O(CO)O— or when taken together with the ring carbon atoms to which they are attached form a double bond;

R⁶ is OR^{a}, N(R^{a})₂, N₃, CN, NO₂, S(O)ₙR^{a}, —C(═O)OR¹¹, —C(═O)NR¹¹R¹², —C(═O)SR¹¹,
═S(O)R¹¹, ═S(O)₂R¹¹, —S(O)(OR¹¹), —S(O)₂(OR¹¹), —SO₂NR¹¹R¹², halogen, (C₁-C₈)alkyl, (C₁-C₈)carbocyclylalkyl, (C₁-C₈)substituted alkyl, (C₂-C₈)alkenyl, (C₂-C₈)substituted alkenyl, (C₂-C₈)alkynyl, (C₂-C₈)substituted alkynyl, or (C₆-C₂₀)aryl(C₁-C₈)alkyl;

R⁷ or R⁹ is selected from a group consisting of
a.) H, —C(═O)R11, —C(═O)NR¹¹R¹², C(═O)SR¹¹, ═S(O)R¹¹, ═S(O)₂R¹¹, —S(O)(OR¹¹), —S(O)₂(OR¹¹), —SO₂NR¹¹R¹², wherein each (C₁-C₈)alkyl, (C₂-C₈)alkenyl, (C₂-C₈)alkynyl, (C₂-C₈)substituted alkynyl, or (C₆-C₂₀)aryl(C₁-C₈)alkyl of each R¹¹ or R¹² is independently, optionally substituted with one or more halo, hydroxy, CN, N₃, N(R^{a})₂, or OR^{a}; and wherein one or more of the non-terminal carbon atoms of each said (C₁-C₈)alkyl may be optionally replaced with —O—, —S—, or NR^{a}—.
b.)

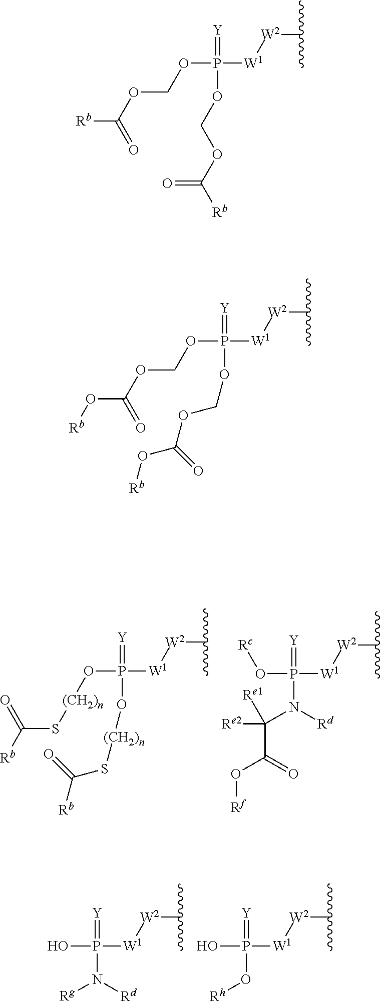

wherein:
   Y is independently, O, S, NR, ⁺N(O)(R), N(OR), ⁺N(O)(OR), or N—NR₂;
   W¹ and W² is each independently O, S, NR, N(OR), CR₂, or C(X⁴)₂;
   R^{b} is (C₁-C₈)alkyl, (C₁-C₈)carbocyclylalkyl, (C₁-C₈)substituted alkyl, or (C₆-C₂₀)aryl(C₁-C₈)alkyl;
   R^{c} is selected from phenyl, 1-naphthyl, 2-naphthyl,

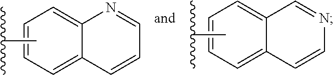

R^{d} is H or CH₃;
R^{e1} and R^{e2} are each independently H, (C₁-C₆)alkyl or benzyl, or halogen
R ^{f} is selected from H, (C₁-C₈)alkyl, benzyl, (C₃-C₆)cycloalkyl, and —CH₂—(C₃-C₆)cycloalkyl;
R^{g} is H, CH₃, (C₁-C₁₂)alkyl, (C₁-C₈)carbocyclylalkyl, (C₁-C₈)substituted alkyl or (C₆-C₂₀)aryl(C₁-C₈)alkyl; alkyl
R^{h} is

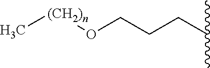

R⁸ is independently NH, or NR

R¹⁰ is independently H, halogen, NR¹¹R¹², N(R¹¹)OR¹¹, N R¹¹N R¹¹1R¹², N₃, NO, NO₂, CHO, CN, —CH(═NR¹¹), —CH═NHNR¹¹, —CH═N(OR¹¹), —CH(OR¹¹)2, —C(═O)NR¹¹R¹², —C(═S)NR¹¹R¹², —C(═OR)OR¹¹, R¹¹, OR¹¹, or SR¹¹;

Each R^{a} is independently H, (C₁-C₈)alkyl, (C₂-C₈) alkenyl, (C₂-C₈)alkynyl, aryl(C₁-C₈)alkyl, (C₄-C₈)carbocyclylalkyl, —C(═O)R¹¹, —C(═O)OR¹¹, —C(═O)NR¹¹R¹², —C(═O)SR¹¹, —S(O)R¹¹, —S(O)₂R¹¹, —S(O)(OR¹¹), —S(O)₂(OR¹¹), or SO₂NR¹¹R¹²;

Each R^{a} is independently H, (C₁-C₈) alkyl, substituted alkyl, (C₂-C₈)alkenyl, (C₂-C₈) substituted alkenyl, (C₂-C₈) substituted alkynyl, C₆-C₂₀ aryl, C₆-C₂₀ substituted aryl, C₂-C₈ heterocyclyl, C₂-C₈ substituted heterocyclyl, arylalkyl, or substituted arylalkyl;

Each n is independently 0, 1, or 2; and

Wherein each (C₁-C₈) alkyl, (C₂-C₈) alkenyl, or aryl(C₁-C₈)alkyl of each R₁, R₂, R₃, R₄, R₅, R₆, is, independently, or optionally substituted with one or more halo, hydroxy, CN, N₃, N(R^{a})₂, or OR^{a}; and wherein one or more of the non-terminal carbon atoms of each said (C₁-C₈) alkyl may be optionally replaced with —O—, —S—, or —NR^{a}—.

R¹¹ or R¹² is independently H, (C₁-C₈)alkyl, (C₂-C₈)alkenyl, (C₁-C₈)carbocyclylalkyl, optionally substituted aryl, optionally substituted heteroaryl, —C(═O)(C₁-C₈)alkyl, —S(O),(C₁-C₈)alkyl or aryl(C₁-C₈)alkyl. In another embodiment, R¹¹ and R¹² taken together with a nitrogen to which they are both attached form a 3 to 7 membered heterocyclic ring wherein any one carbon atom of said heterocyclic ring can optionally be replaced with —O—, —S—, or —NR^{a}—. Therefore, by way of example and not limitation, the moiety —NR¹¹R¹² can be represented by the heterocycles:

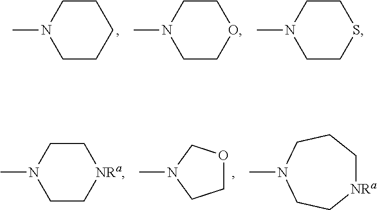

and the like.
Each X¹ or X² is independently C, C—R¹³, or N.

In another aspect, the present invention includes compounds of Formula I and pharmaceutically acceptable salts thereof and all racemates, enantiomers, diastereomers, tautomers, polymorphs, pseudopolymorphs and amorphous form, hydrate, solvate, or ester thereof.

In another aspect, the present invention provides novel compounds of Formula I with activity against infectious Coronaviridae viruses. Without wishing to be bound by theory, the compounds of this invention may inhibit viral RNA-dependent RNA polymerase and thus inhibit the replication of the virus. They are useful for treating human patients infected with a human virus such as SARS-CoV-2.

In another aspect, the invention the invention provides a pharmaceutical composition comprising an effective amount of Formula I compound, or a pharmaceutically acceptable salt thereof, in combination with a pharmaceutically acceptable diluent or carrier.

In another embodiment, the present application provides for combination pharmaceutical agent comprising:

a) a first pharmaceutical composition comprising a compound of Formula I; or a pharmaceutically acceptable salt, solvate, or ester thereof; and

b) a second pharmaceutical composition comprising at least one additional therapeutic agent selected form the group consisting of interferons, monoclonal antibodies, 3CL protease inhibitors, corticosteroids, or at least one additional therapeutic agent active against infectious Coronaviridae viruses

In another embodiment, the present application provides for a method of inhibiting Coronaviridae RNA-dependent RNA polymerase, comprising contacting a cell infected with Coronaviridae virus with an effective amount of compound of Formula I; or a pharmaceutically acceptable salt, solvate, and/or ester thereof.

In another embodiment, the present application provides for a method of inhibiting Coronaviridae RNA-dependent RNA polymerase, comprising contacting a cell infected with Coronaviridae virus with an effective amount of compound of Formula I; or a pharmaceutically acceptable salt, solvate, and/or ester thereof; and at least one additional therapeutic agent.

In another embodiment, the present application provides for a method of treating and/or preventing a disease caused by a viral infection wherein the viral infection is caused by a virus selected from the group consisting of dengue virus, yellow fever virus, West Nile virus, Japanese encephalitis virus, St. Louis encephalitis virus, Omsk hemorrhagic fever, Ebola virus, bovine viral diarrhea virus, Zika virus, Marburg virus, Hepatitis C virus, human coronavirus 229E, human coronavirus 0C_{43,} Middle East Respiratory Syndrome virus, Severe Acute Respiratory Syndrome virus, and Severe Acute Respiratory Syndrome virus 2; by administering to a subject in need thereof a therapeutically effective amount of a compound of Formula I or a pharmaceutically acceptable salt thereof.

In another embodiment, the present application provides a method of treating Coronaviridae infection in a human in need thereof, comprising administering a therapeutically effective amount of a pharmaceutical composition comprising an effective amount of Formula I compound; or a pharmaceutically acceptable salt, solvate, and/or ester thereof, in combination with a pharmaceutically acceptable diluent or carrier.

In another embodiment, the present application provides a method of treating Coronaviridae infection in a human in need thereof, comprising administering a therapeutically effective amount of a pharmaceutical composition comprising an effective amount of Formula I compound; or a pharmaceutically acceptable salt, solvate, and/or ester thereof, in combination with at least one additional therapeutic agent.

Another aspect of the invention provides a method for the treatment or prevention of the symptoms or effects of a Coronaviridae infection in an infected animal which comprises administering to, i.e. treating said animal with a pharmaceutical combination composition or formulation comprising an effective amount of a Formula I compound, and a second compound having anti-Coronaviridae properties.

In another aspect, the invention also provides a method of inhibiting Coronaviridae virus, comprising administering to a mammal infected with Coronaviridae virus an amount of a Formula I or Formula II compound, effective to inhibit the replication of SARS-CoV-2 in infected cells in said mammal.

In another aspect, the invention also provides processes and novel intermediates disclosed herein which are useful for preparing Formula I compounds of the invention.

In other aspects, novel methods for synthesis, analysis, separation, isolation, purification, characterization and testing of the compounds of this invention are provided.

In another aspect, compounds of Formula I are represented by Formula II:

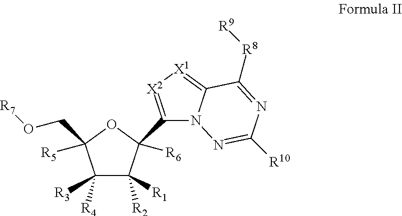

or a or a pharmaceutically acceptable salt or ester thereof;
wherein:

each R¹ is H or halogen

each R², R³, R⁴, or R⁵ is independently H, OR^{a}, N(R^{a})₂, N₃, CN, NO₂, S(O)ₙR^{a}, halogen, (C₁-C₈)alkyl, (C₄-C₈)carbocyclalkyl, (C₁-C₈) substituted alkyl, (C₂-C₈) alkenyl, (C₂-C₈)substituted alkenyl, (C₂-C₈)alkynyl, (C₂-C₈)substituted alkynyl or aryl(C₂-C₈)alkyl;
or any two R¹, R², R³, R⁴, or R⁵ on adjacent carbon atoms when taken together are —O(CO)O— or when taken together with the ring carbon atoms to which they are attached form a double bond;

R⁶ is OR^{a}, N(R^{a})₂, N₃, CN, NO₂, S(O)ₙR^{a}, —C(═O)OR¹¹, —C(═O)NR¹¹R¹², —C(═O)SR¹¹, ═S(O)R¹¹, ═S(O)₂R¹¹, —S(O)(OR¹¹), —S(O)₂(OR¹¹), —SO₂NR¹¹R¹², halogen, (C₁-C₈)alkyl, (C₁-C₈)carbocyclylalkyl, (C₁-C₈)substituted alkyl, (C₂-C₈)alkenyl, (C₂-C₈)substituted alkenyl, (C₂-C₈)alkynyl, (C₂-C₈)substituted alkynyl, or (C₆-C₂₀)aryl(C₁-C₈)alkyl;

R⁷ or R⁹ is selected from a group consisting of
a.) H, —C(═O)R¹¹, —C(═O)NR¹¹R¹², C(═O)SR¹¹, ═S(O)R¹¹, ═S(O)₂R¹¹, —S(O)(OR¹¹), —S(O)₂(OR¹¹), —SO₂NR¹¹R¹², wherein each (C₁-C₈)alkyl, (C₂-C₈)alkenyl, (C₂-C₈)alkynyl, (C₂-C₈)substituted alkynyl, or (C₆-C₂₀)aryl(C₁-C₈)alkyl of each R¹¹ or R¹² is independently, optionally substituted with one or more halo, hydroxy, CN, N₃, N(R^{a})₂, or OR^{a}; and wherein one or more of the non-terminal carbon atoms of each said (C₁-C₈)alkyl may be optionally replaced with —O—, —S—, or NR^{a},
b.)

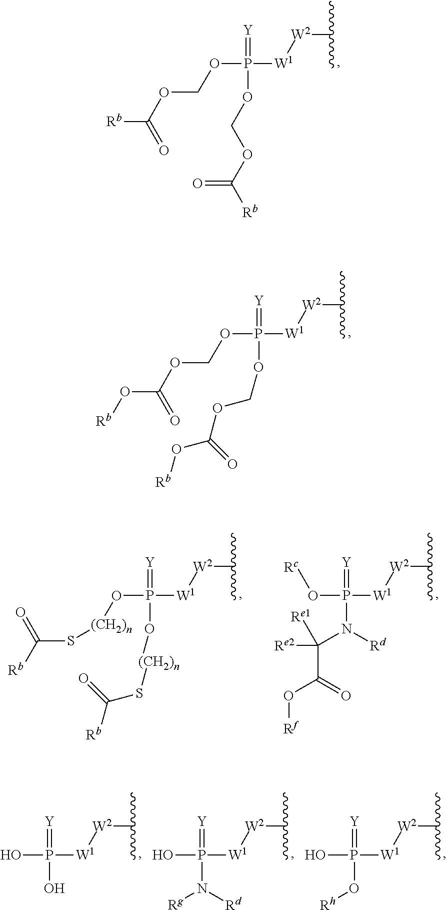

and
c.)

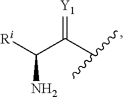

wherein:
   Y and Y¹ is independently, O, S, NR, ⁺N(O)(R), N(OR), ⁺N(O)(OR), or N—NR₂;
   W¹ and W² is each independently O, S, NR, N(OR), CR₂, or C(X⁴)₂;
   R^{b} is (C₁-C₈)alkyl, (C₁-C₈)carbocyclylalkyl, (C₁-C₈)substituted alkyl, or (C₆-C₂₀)aryl(C₁-C₈)alkyl;
   R^{c} is selected from phenyl, 1-naphthyl, 2-naphthyl,

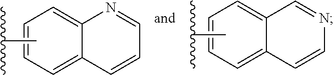

R^{d} is H or CH₃;
R^{e1} and R^{e2} are each independently H, (C₁-C₆)alkyl or benzyl, or halogen
R^{f} is selected from H, (C₁-C₈)alkyl, benzyl, (C₃-C₆)cycloalkyl, and —CH₂—(C₃-C₆)cycloalkyl;
R^{g} is H, CH₃, (C₁-C₁₂)alkyl,(C₁-C₈)carbocyclylalkyl, (C₁-C₈)substituted alkyl or (C₆-C₂₀)aryl(C₁-C₈)alkyl; alkyl
R^{h} is

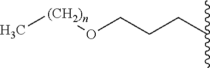

Rⁱ is (C₁-C₈)alkyl, (C₁-C₈)substituted alkyl, (C₁-C₈)carbocyclylalkyl, or (C₆-C₂₀)aryl(C₁-C₈)alkyl

R⁸ is independently NH, or NR

R¹⁰ is independently H, halogen, NR¹¹R¹², N(R¹¹)OR¹¹, N R¹¹N R¹¹1R¹², N₃, NO, NO₂, CHO, CN, —CH(═NR11), —CH═NHNR¹¹, —CH═N(OR₁₁), —CH(OR₁₁)2, —C(═O)NR¹¹R¹², —C(═S)NR¹¹R¹², —C(═OR)OR¹¹, R¹¹, OR¹¹, or SR¹¹;

Each R^{a} is independently H, (C₁-C₈)alkyl, (C₂-C₈) alkenyl, (C₂-C₈)alkynyl, aryl(C₁-C₈)alkyl, (C₄-C₈)carbocyclylalkyl, —C(═O)R¹¹, —C(═O)OR¹¹, —C(═O)NR¹¹R¹², —C(═O)SR¹¹, —S(O)R¹¹, —S(O)₂R¹¹, —S(O)(OR¹¹), —S(O)₂(OR¹¹), or SO₂NR¹¹R¹²;

Each R^{a} is independently H, (C₁-C₈) alkyl, substituted alkyl, (C₂-C₈)alkenyl, (C₂-C₈) substituted alkenyl, (C₂-C₈) substituted alkynyl, C₆-C₂₀ aryl, C₆-C₂₀ substituted aryl, C₂-C₈ heterocyclyl, C₂-C₈ substituted heterocyclyl, arylalkyl, or substituted arylalkyl;

Each n is independently 0, 1, or 2; and

Wherein each (C₁-C₈) alkyl, (C₂-C₈) alkenyl, or aryl(C₁-C₈)alkyl of each R₁, R₂, R₃, R₄, R₅, R₆, is, independently, or optionally substituted with one or more halo, hydroxy, CN, N₃, N(R^{a})₂, or OR^{a}; and wherein one or more of the non-terminal carbon atoms of each said (C₁-C₈) alkyl may be optionally replaced with —O—, —S—, or —NR^{a}—.

Each X¹ or X² is independently C, C-R¹³, or N.

In one embodiment of the invention of Formula II, R¹ is (C₁-C₈) alkyl, (C₁-C₈) substituted alkyl, (C₂-C₈) alkenyl, (C₂-C₈)substituted alkenyl, (C₂-C₈)alkynyl, or (C₂-C₈)substituted alkynyl. In another aspect of this embodiment, R¹ is (C₁-C₈) alkyl. In one aspect of this embodiment, R¹ is H. In a preferred aspect of this embodiment, R¹ is H.

In one embodiment of the invention of Formula II, R² is H, OR^{a}, N(R^{a})₂, N₃, CN, NO₂, S(O)ₙR^{a}, halogen, (C₁-C₈)alkyl, (C₄-C₈)carbocyclalkyl, (C₁-C₈) substituted alkyl, (C₂-C₈) alkenyl, (C₂-C₈)substituted alkenyl, (C₂-C₈)alkynyl, (C₂-C₈)substituted alkynyl or aryl(C₂-C₈)alkyl. In one aspect of this embodiment, R² is OR^{a} or OH. In a preferred aspect of this embodiment, R² is OR^{a} and R¹ is H. In another preferred aspect of this embodiment, R² is OH and R¹ is H.

In one embodiment of Formula II, R³ is H, OR^{a}, N(R^{a})₂, N₃, CN, NO₂, S(O)ₙR^{a}, halogen, (C₁-C₈)alkyl, (C₄-C₈)carbocyclalkyl, (C₁-C₈) substituted alkyl, (C₂-C₈) alkenyl, (C₂-C₈)substituted alkenyl, (C₂-C₈)alkynyl, (C₂-C₈)substituted alkynyl or aryl(C₂-C₈)alkyl. In one aspect of this embodiment, R³ is H. In a preferred aspect of this embodiment, R³ is H, R² is OR^{a} and R¹ is H. In another preferred aspect of this embodiment, R³ is H, R² is OH and R¹ is H.

In one embodiment of Formula II, R⁴ is H, OR^{a}, N(R^{a})₂, N₃, CN, NO₂, S(O)ₙR^{a}, halogen, (C₁-C₈)alkyl, (C₄-C₈)carbocyclalkyl, (C₁-C₈)substituted alkyl, (C₂-C₈) alkenyl, (C₂-C₈)substituted alkenyl, (C₂-C₈)alkynyl, (C₂-C₈)substituted alkynyl or aryl(C₂-C₈)alkyl. In one aspect of this embodiment, R⁴ is OR^{a} or OH. In a preferred aspect of this embodiment, R⁴ is OR^{a}, R³ is H, R² is OR^{a} and R¹ is H. In another preferred aspect of this embodiment, R⁴ is OH, R³ is H, R² is OR^{a} and R¹ is H. In another preferred aspect of this embodiment, R⁴ is OH, R³ is H, R² is OH and R¹ is H.

In one embodiment of Formula II, R⁵ is H, OR^{a}, N(R^{a})₂, N₃, CN, NO₂, S(O)ₙR^{a}, halogen, (C₁-C₈)alkyl, (C₄-C₈)carbocyclalkyl, (C₁-C₈) substituted alkyl, (C₂-C₈) alkenyl, (C₂-C₈)substituted alkenyl, (C₂-C₈)alkynyl, (C₂-C₈)substituted alkynyl or aryl(C₂-C₈)alkyl. In one aspect of this embodiment, R⁵ is H. In a preferred aspect of this embodiment, R⁵ is H, R⁴ is OR^{a}, R³ is H, R² is OR^{a} and R¹ is H. In another preferred aspect of this embodiment, R⁵ is H, R⁴ is OH, R³ is H, R² is OR^{a} and R¹ is H. In another preferred aspect of this embodiment, R⁵ is H, R⁴ is OR^{a}, R³ is H, R² is OH and R¹ is H. In another preferred aspect of this embodiment, R⁵ is H, R⁴ is OH, R³ is H, R² is OH and R¹ is H.

In one embodiment of Formula II, R⁶ is OR^{a}, N(R^{a})₂, N₃, CN, NO₂, S(O)ₙR^{a}, —C(═O)OR¹¹, —C(═O)NR¹¹R¹², —C(═O)SR¹¹, ═S(O)R¹¹, ═S(O)₂R¹¹, —S(O)(OR¹¹) —S(O)₂(OR¹¹), —SO₂NR¹¹R¹², halogen, (C₁-C₈)alkyl, (C₁-C₈)carbocyclylalkyl, (C₁-C₈)substituted alkyl, (C₂-C₈)alkenyl, (C₂-C₈)substituted alkenyl, (C₂-C₈)alkynyl, (C₂-C₈)substituted alkynyl, or (C₆-C₂₀)aryl(C₁-C₈)alkyl. In one aspect of this embodiment, R⁶ is CN. In a preferred aspect of this embodiment, R⁶ is CN, R⁵ is H, R⁴ is OR^{a}, R³ is H, R² is OR^{a} and R¹ is H. In another preferred aspect of this embodiment, R⁶ is CN, R⁵ is H, R⁴ is OH, R³ is H, R² is OR^{a} and R¹ is H. In another preferred aspect of this embodiment, R⁶ is CN, R⁵ is H, R⁴ is OR^{a}, R³ is H, R² is OH and R¹ is H. In another preferred aspect of this embodiment, R⁶ is CN, R⁵ is H, R⁴ is OH, R³ is H, R² is OH and R¹ is H.

In one embodiment of Formula II, R⁷ is —C(═O)R¹¹,

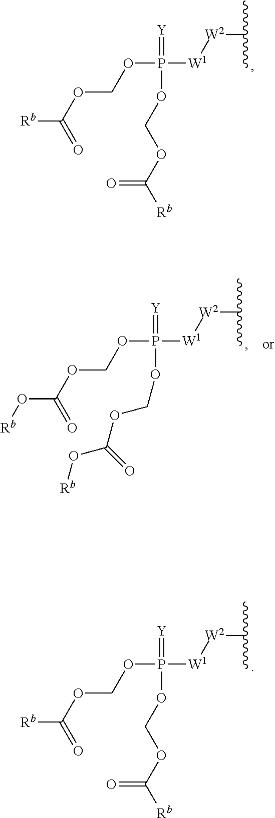

In a preferred aspect of this embodiment, R⁷ is H. In another preferred aspect of this embodiment, R⁷ is —C(═O)R¹¹. In another preferred aspect of this embodiment, R⁷ is —C(═O)R¹¹ wherein R¹¹ is (C₁-C₈)alkyl. In another preferred aspect of this embodiment, R⁷ is —C(═O)R¹¹ wherein R¹¹ is (C₁-C₈)substituted alkyl.

In another preferred aspect of this embodiment, R⁷ is

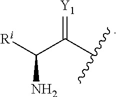

In another preferred aspect of this embodiment, R⁷ is

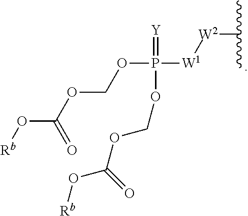

In another preferred aspect of this embodiment, R⁷ is

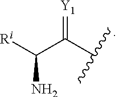

In another preferred aspect of this embodiment, R⁷ is

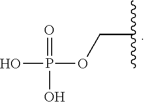 [SMILES: *COP(=O)(O)O]

In one embodiment of Formula II, X¹ is C—R¹³. In one aspect of this embodiment, X¹ is C—R¹³, wherein R¹³ is H. In another aspect of this embodiment, X² is C—H. In another aspect of this embodiment, X¹ is C—H and X² is C—H. In another aspect of this embodiment, X¹ is C—H, X² is C—H, R⁶ is CN, R⁵ is H, R⁴ is OR^{a}, R³ is H, R² is OR^{a} and R¹ is H. In another aspect of this embodiment, X¹ is C—H, X² is C—H, R⁶ is CN, R⁵ is H, R⁴ is OH, R³ is H, R² is OR^{a} and R¹ is H. In another aspect of this embodiment, X¹ is C—H, X² is C—H, R⁶ is CN, R⁵ is H, R⁴ is OR^{a}, R³ is H, R² is OH and R¹ is H. In another aspect of this embodiment, X¹ is C—H, X² is C—H, R⁶ is CN, R⁵ is H, R⁴ is OH, R³ is H, R² is OH and R¹ is H.

In another embodiment of Formula II, each R⁸ is independently NH or NR. In one aspect of this embodiment, R⁸ is NH. In another aspect of this embodiment, R⁸ is independently NH and R⁹ is H,

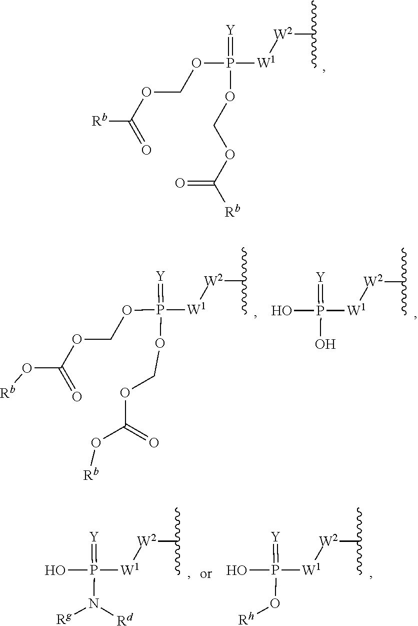

In a preferred aspect of this embodiment R⁸ is NH and R⁹ is H.

In another embodiment of Formula II, each R¹⁰ is independently H, halogen, NR¹¹R¹², N(R¹¹)OR¹¹, N R¹¹N R¹¹1R¹², N₃, NO, NO₂, CHO, CN, —CH(═NR¹¹), —CH═NHNR¹¹, —CH═N(OR¹¹), —CH(OR¹¹)2, —C(═O)NR¹¹R¹², C(═S)NR¹¹R¹², C(═OR)OR¹¹, R¹¹, OR¹¹, or SR¹¹. In another aspect of this embodiment, each R¹⁰ is H. In a preferred aspect of this embodiment, R¹⁰ is H.

In one embodiment of Formulas I-II, R¹¹ or R¹² is independently H, (C₁-C₈)alkyl, (C₂-C₈)alkenyl, (C₁-C₈)carbocyclylalkyl, optionally substituted aryl, optionally substituted heteroaryl, —C(═O)(C₁-C₈)alkyl, —S(O)ₙ(C₁-C₈)alkyl or aryl(C₁-C₈)alkyl. In another embodiment, R¹¹ and R¹² taken together with a nitrogen to which they are both attached form a 3 to 7 membered heterocyclic ring wherein any one carbon atom of said heterocyclic ring can optionally be replaced with —O—, —S—, or —NR^{a}—. Therefore, by way of example and not limitation, the moiety —NR¹¹R¹² can be represented by the heterocycles:

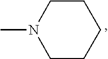

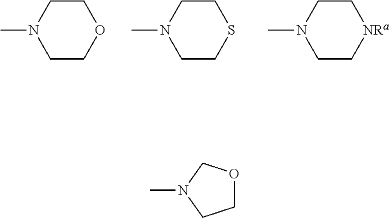

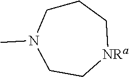

and the like.

In another embodiment of Formulas I-II, R^{2′}, R³, R⁴, or R⁵, R⁶, R¹¹ or R¹² is independently (C₁-C₈)alkyl, (C₂-C₈)alkenyl, (C₂-C₈)alkynyl, or aryl(C₁-C₈)alkyl, wherein said (C₁-C₈)alkyl, (C₂-C₈)alkenyl, (C₂-C₈)alkynyl, or aryl(C₁-C₈)alkyl are, independently, optionally substituted with one or more halo, hydroxy, CN, N₃, N(R^{a})₂ or OR^{a}. Therefore, by way of example and not limitation, R², R³, R⁴, or R⁵, R⁶, R¹¹ or R¹² is (C₁-C₈)alkyl wherein one or more of the non-terminal carbon atoms of each said (C₁-C₈)alkyl may be optionally replaced with —O—, —S—, or —NR^{a}—. Therefore, by way of example and not limitation, R², R³, R⁴, or R⁵, R⁶, R¹¹ or R¹² could represent moieties such as —CH₂OCH₃, —CH₂OCH₂CH₃, —CH₂OCH(CH₃)₂, —CH₂SCH₃, —(CH₂)₆OCH₃, —(CH₂)₆N(CH₃)₂ and the like.

In still another embodiment, the compounds of Formula I or Formula II are named below in tabular format (Table 6) as compounds of general Formula III:

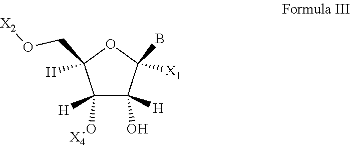

wherein X1 represents a substituent attached to the tetrahydrofuranyl ring as defined in Table 1 below; X2 represents a substituent attached to the primary alcohol as defined in Table 2 below; B is a purine defined in Table 5, below; and X3 represents a substituent attached to the amine of the purine base B as described in Table 3 below.

The point of attachment of the core structure ribose is indicated in each of the structures of X1, X2, and B. The point of attachment of the core structure purine is indicated in each of the structures X3. The point of attachment at the 3′ hydroxyl group on the ribose is indicated in each of the structures X4. Each structure in Tables 1-4 is represented by an alphanumeric “code”. Each structure of a compound of Formula III can thus be designated in tabular form by combining the “code” representing each structural moiety using the following syntax: X1.X2.B. Thus, for example, X1a.X2c.X3a.X4a.B1 represents the following structure:

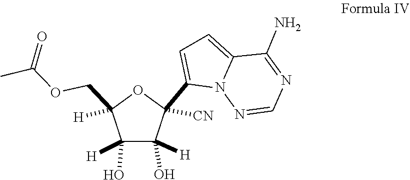 [SMILES: CC(=O)OC[C@H]1O[C@@](C#N)(c2ccc3c(N)ncnn23)[C@H](O)[C@@H]1O]

TABLE 1
X1 Structures

| Code | Structure |
|---|---|
| X1a | CN |

TABLE 2
X2 Structures

| Code | Structure |
|---|---|
| X2a | 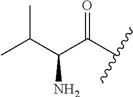 [SMILES: *C(=O)[C@@H](N)C(C)C] |
| X2b | 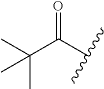 [SMILES: *C(=O)C(C)(C)C] |
| X2c | 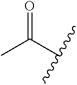 [SMILES: *C(C)=O] |
| X2d | 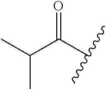 [SMILES: *C(=O)C(C)C] |
| X2e | 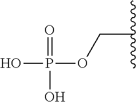 [SMILES: *COP(=O)(O)O] |
| X2f | 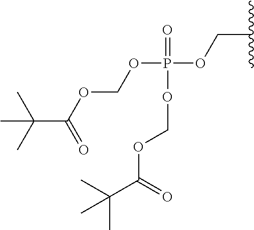 [SMILES: *COP(=O)(OCOC(=O)C(C)(C)C)OCOC(=O)C(C)(C)C] |
| X2g | 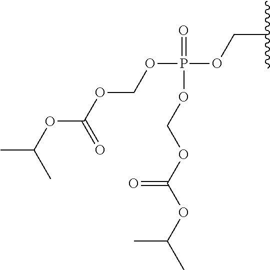 [SMILES: *COP(=O)(OCOC(=O)OC(C)C)OCOC(=O)OC(C)C] |
| X2h | 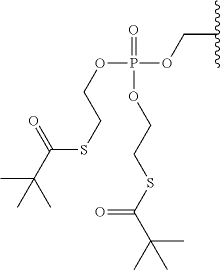 [SMILES: *COP(=O)(OCCSC(=O)C(C)(C)C)OCCSC(=O)C(C)(C)C] |
| X2i | 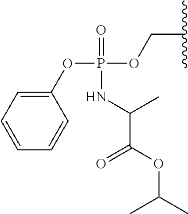 [SMILES: *COP(=O)(NC(C)C(=O)OC(C)C)Oc1ccccc1] |
| X2j | 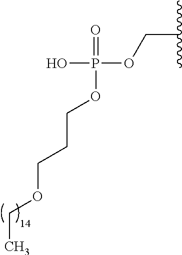 |
| X2k | 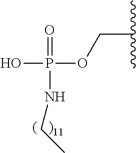 |
| X2l | 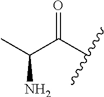 [SMILES: *C(=O)[C@H](C)N] |
| X2m | 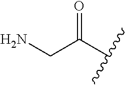 [SMILES: *C(=O)CN] |
| X2n | 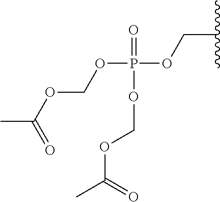 [SMILES: *COP(=O)(OCOC(C)=O)OCOC(C)=O] |

TABLE 3
X3 Structures

| Code | Structure |
|---|---|
| X3a | H |
| X3b | 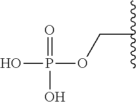 [SMILES: *COP(=O)(O)O] |
| X3c | 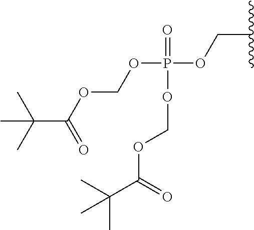 [SMILES: *COP(=O)(OCOC(=O)C(C)(C)C)OCOC(=O)C(C)(C)C] |
| X3d | 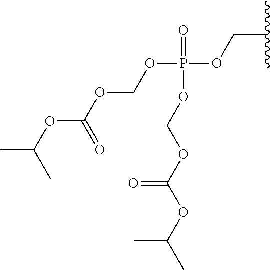 [SMILES: *COP(=O)(OCOC(=O)OC(C)C)OCOC(=O)OC(C)C] |
| X3e | 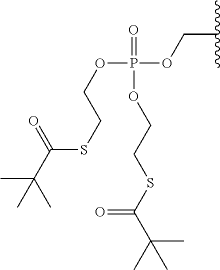 [SMILES: *COP(=O)(OCCSC(=O)C(C)(C)C)OCCSC(=O)C(C)(C)C] |
| X3f | 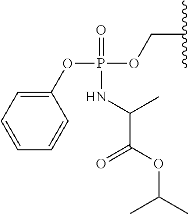 [SMILES: *COP(=O)(NC(C)C(=O)OC(C)C)Oc1ccccc1] |
| X3g | 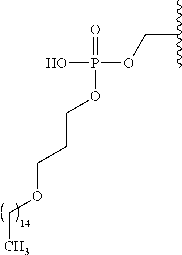 |
| X3h | 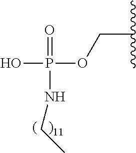 |

TABLE 4
X4 Structures

| Code | Structure |
|---|---|
| X4a | H |
| X4b | 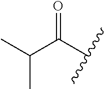 [SMILES: *C(=O)C(C)C] |

TABLE 5
B Structures

| Code | Structure |
|---|---|
| B1 | 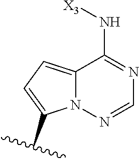 |

TABLE 6
List of Compounds of Formula III

| | | |
|---|---|---|
| X1a.X2a.X3a.X4a.B1 | X1a.X2b.X3a.X4a.B1 | X1a.X2c.X3a.X4a.B1 |
| X1a.X2d.X3a.X4a.B1 | X1a.X2e.X3a.X4a.B1 | X1a.X2f.X3a.X4a.B1 |
| X1a.X2g.X3a.X4a.B1 | X1a.X2h.X3a.X4a.B1 | X1a.X2i.X3a.X4a.B1 |
| X1a.X2j.X3a.X4a.B1 | X1a.X2k.X3a.X4a.B1 | X1a.X2l.X3a.X4a.B1 |
| X1a.X2m.X3a.X4a.B1 | X1a.X2n.X3a.X4a.B1 | X1a.X2d.X3a.X4b.B1 |

In another embodiment, Formulas I-II is a compound selected from the group consisting of

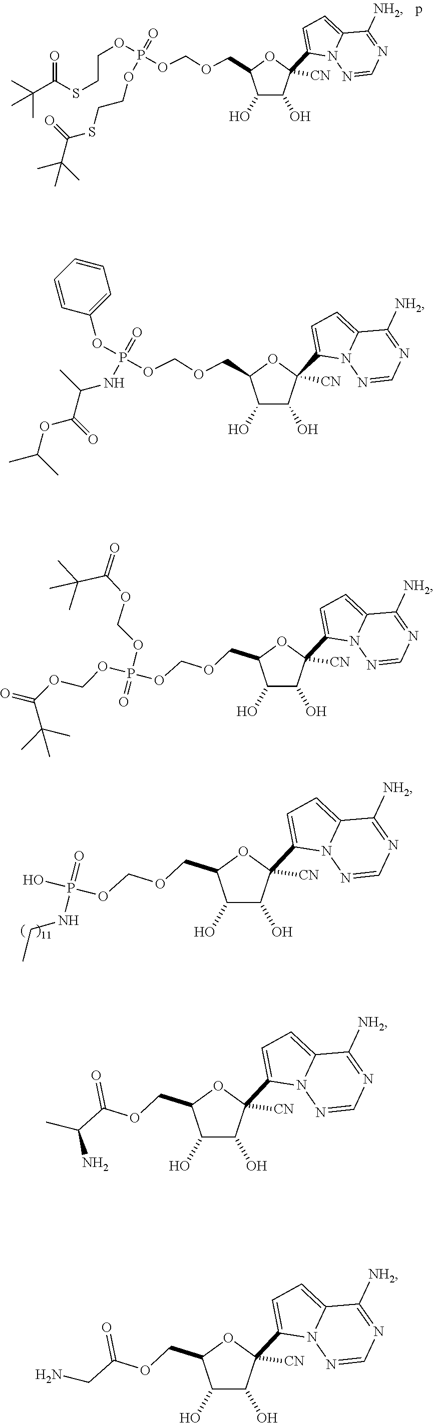

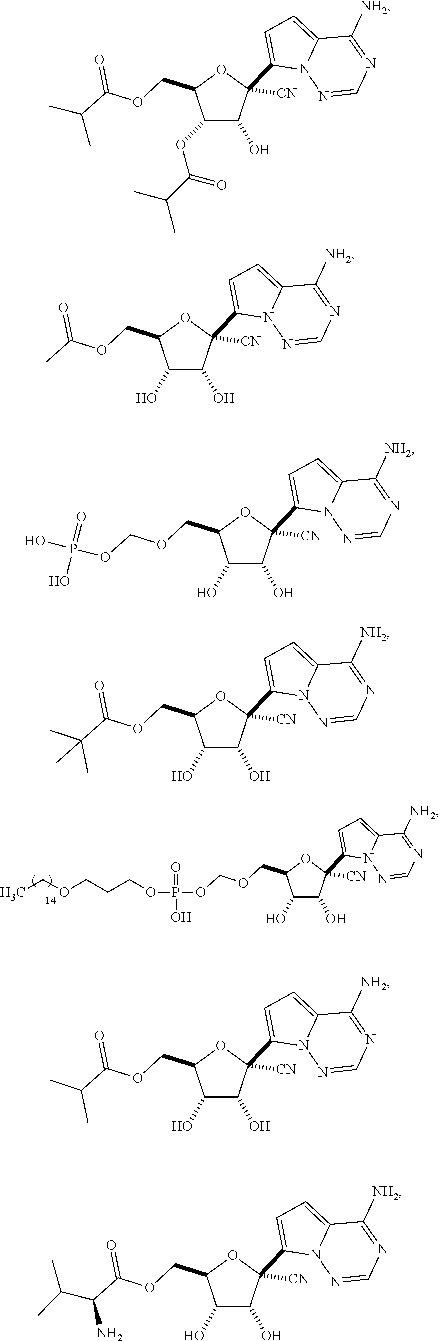

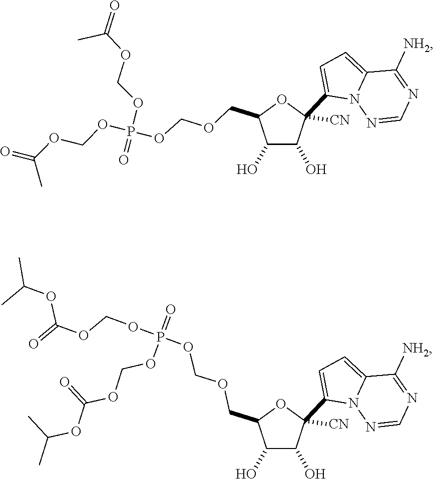

or a pharmaceutically acceptable salt thereof.

The compound of the present invention has a structure of Formula III, wherein X2 is selected from the group consisting of X2a, X2b, X2c and X2d as shown in Table 2. For example, the compound may be selected from the group of compounds consisting of X1a.X2a.X3a.X4a.B1, X1a.X2a.X3a.X4b.B1, X1a.X2a.X3b.X4a.B1, X1a.X2a.X3b.X4b.B1, X1a.X2a.X3c.X4a.B1, X1a.X2a.X3c.X4b.B1, X1a.X2a.X3d.X4a.B1, X1a.X2a.X3d.X4b.B1, X1a.X2a.X3e.X4a.B1, X1a.X2a.X3e.X4b.B1, X1a.X2a.X3f.X4a.B1, X1a.X2a.X3f.X4b.B1, X1a.X2a.X3g.X4a.B1, X1a.X2a.X3g.X4b.B1, X1a.X2a.X3h.X4a.B1, X1a.X2a.X3h.X4b.B1, X1a.X2b.X3a.X4a.B1, X1a.X2b.X3a.X4b.B1, X1a.X2b.X3b.X4a.B1, X1a.X2b.X3b.X4b.B1, X1a.X2b.X3c.X4a.B1, X1a.X2b.X3c.X4b.B1, X1a.X2b.X3d.X4a.B1, X1a.X2b.X3d.X4b.B1, X1a.X2b.X3e.X4a.B1, X1a.X2b.X3e.X4b.B1, X1a.X2b.X3f.X4a.B1, X1a.X2b.X3f.X4b.B1, X1a.X2b.X3g.X4a.B1, X1a.X2b.X3g.X4b.B1, X1a.X2b.X3h.X4a.B1, X1a.X2b.X3h.X4b.B1, X1a.X2c.X3a.X4a.B1, X1a.X2c.X3a.X4b.B1, X1a.X2c.X3b.X4a.B1, X1a.X2c.X3b.X4b.B1, X1a.X2c.X3c.X4a.B1, X1a.X2c.X3c.X4b.B1, X1a.X2c.X3d.X4a.B1, X1a.X2c.X3d.X4b.B1, X1a.X2c.X3e.X4a.B1, X1a.X2c.X3e.X4b.B1, X1a.X2c.X3f.X4a.B1, X1a.X2c.X3f.X4b.B1, X1a.X2c.X3g.X4a.B1, X1a.X2c.X3g.X4b.B1, X1a.X2c.X3h.X4a.B1, X1a.X2c.X3h.X4b.B1, X1a.X2d.X3a.X4a.B1, X1a.X2d.X3a.X4b.B1, X1a.X2d.X3b.X4a.B1, X1a.X2d.X3b.X4b.B1, X1a.X2d.X3c.X4a.B1, X1a.X2d.X3c.X4b.B1, X1a.X2d.X3d.X4a.B1, X1a.X2d.X3d.X4b.B1, X1a.X2d.X3e.X4a.B1, X1a.X2d.X3e.X4b.B1, X1a.X2d.X3f.X4a.B1, X1a.X2d.X3f.X4b.B1, X1a.X2d.X3g.X4a.B1, X1a.X2d.X3g.X4b.B1, X1a.X2d.X3h.X4a.B1 and X1a.X2d.X3h.X4b.B1. For example, the compound may be X1a.X2a.X3a.X4a.B1, X1a.X2b.X3a.X4a.B1, X1a.X2c.X3a.X4a.B1, X1a.X2d.X3a.X4a.B1 or X1a.X2d.X3a.X4b.B1.

In one embodiment, the compound of the present invention has the structure of Formula V, which is also referred to as CAT002 and corresponds to X1a.X2a.X3a.X4a.B1 of Formula III:

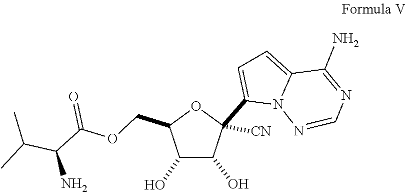

### I. DEFINITIONS

Unless stated otherwise, the following terms and phrases as used herein are intended to have the following meanings:

When trade names are used herein, applicants intend to independently include the trade name product and the active pharmaceutical ingredient(s) of the trade name product.

As used herein, a “compound of the invention” or a “compound of Formula I” means a compound of Formula I or a pharmaceutically acceptable salt, thereof. Similarly, with respect to isolatable intermediates, the phrase “a compound of Formula (number)” means a compound of that formula and pharmaceutically acceptable salts, thereof.

“Alkyl” is a hydrocarbon containing normal, secondary, tertiary, or cyclic carbon atoms. For example, an alkyl group can have 1 to 20 carbon atoms (i.e., C₁-C₂₀ alkyl), 1 to 8 carbon atoms (i.e., C₁-C₈ alkyl), or 1 to 6 carbon atoms (i.e., C₁-C₆ alkyl). Examples of suitable alkyl groups include, but are not limited to methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂ CH₂ CH₂CH₃), 2-methyl-1-propyl (i-Bu, i-butyl, —CH₂CH(CH₃)₂), 2-butyl (s-Bu, s-butyl, —CH(CH₃) CH₂CH₃), 2-methyl-2-propyl (t-Bu, t-butyl, —C(CH₃)₃), 1-pentyl (n-pentyl, —CH₂CH₂CH₂ CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (1CH₂CH₂CH₂CH₂CH₂CH₃), 2-hexyl (—CH(CH₃)CH₂CH₂CH₂CH₃), 3-hexyl (—CH(CH₂CH₃) (CH₂CH₂CH₃)), 2-methyl-2pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)CH(CH₃)₂), 3,3-dimethyl-2-butyl (—CH(CH₃)C(CH₃)₃), and octyl (—(CH₂)₇CH₃).

“Alkoxy” means a group having the formula —O-alkyl, in which an alkyl group, as defined above, is attached to the parent molecule via an oxygen atom. The alkyl portion of an alkoxy group can have 1 to 20 carbon atoms (i.e., C₁-C₂₀ alkoxy), 1 to 12 carbon atoms (i.e., C₁-C₁₂ alkoxy), or 1 to 6 carbon atoms (i.e., C₁-C₆ alkoxy). Examples of suitable alkoxy groups include, but are not limited to, methoxy (—O—CH₃ or —OMe), ethoxy (—OCH₂CH₃ or —OEt), t-butoxy (—O—C(CH₃)₃ or —OtBu) and the like.

“Haloalkyl” is an alkyl group, as defined above, in which one or more hydrogen atoms of the alkyl group is replaced with a halogen atom. The alkyl portion of a haloalkyl group can have 1 to 20 carbon atoms (i.e., C₁-C₂₀ haloalkyl) 1 to 12 carbon atoms (i.e., C₁-C₁₂ haloalkyl), or 1 to 6 carbon atoms (i.e., C₁-C₆ haloalkyl). Examples of suitable haloalkyl groups include, but are not limited to —CF₃, —CHF₂, —CFH₂, —CH₂CF₃, and the like.

“Alkenyl” is a hydrocarbon containing normal, secondary, tertiary, or cyclic carbon atoms with at least one site of unsaturation, i.e. a carbon-carbon sp² double bond. For example, an alkenyl group can have 2 to 20 carbon atoms (i.e., C₂-C₂₀ alkenyl), 2 to 8 carbon atoms (i.e., C₂-C₈ alkenyl), or 2 to 6 carbon atoms (i.e., C₂-C₆ alkenyl). Examples of suitable alkenyl groups include, but are not limited to, ethylene or vinyl (—CH═CH₂), allyl (—CH₂CH═CH₂), cyclopentyl (—C₅H₇), and 5-hexenyl (—CH₂CH₂CH₂CH₂CH₂═CH₂).

“Alkynyl” is a hydrocarbon containing normal, secondary, tertiary or cyclic carbon atoms with at least one site of unsaturation, i.e. a carbon-carbon sp triple bond. For example, an alkynyl group can have at least 2 to 20 carbon atoms (i.e., C₂-C₂₀ alkynyl), 2 to 8 carbon atoms(i.e., C₂-C₈ alkynyl), or 2 to 6 carbon atoms (i.e., C₂-C₆ alkynyl). Examples of suitable alkynyl groups include, but are not limited to, acetylenic (—C≡CH), propargyl (—CH₂C≡CH), and the like.

“Alkylene” refers to a saturated, branched or straight chain or cyclic hydrocarbon radical having two monovalent radical centers derived by the removal of two hydrogen atoms from the same or two different carbon atoms of a parent alkane. For example, an alkylene group can have 1 to 20 carbon atoms, 1 to 10 carbon atoms, or 1 to 6 carbon atoms. Typical alkylene radicals include, but are not limited to, methylene (—CH₂—), 1,1-ethyl (—CH(CH₃)—), 1,2-ethyl (—CH₂CH₂—), 1,1-propyl (—CH(CH₂CH₃)—, 1,2-propyl (—CH₂CH(CH₃)—), 1,3-propyl (—CH₂CH₂CH₂—), 1,4-butyl (—CH₂CH₂CH₂CH₂—), and the like.

“Alkenylene” refers to an unsaturated, branched or straight chain or cyclic hydrocarbon radical having two monovalent radical centers derived by the removal of two hydrogen atoms from the same or two different carbon atoms of a parent alkene. For example, an alkenylene group can have 1 to 20 carbon atoms, 1 to 10 carbon atoms, or 1 to 6 carbon atoms. Typical alkenylene radicals include, but are not limited to 1,2-ethylene (—CH═CH—).

“Alkynylene” refers to an unsaturated, branched or straight chain or cyclic hydrocarbon radical having two monovalent radical centers derived by the removal of two hydrogen atoms from the same or two different carbon atoms of a parent alkyne. For example, an alkynylene group can have 1 to 20 carbon atoms, 1 to 10 carbon atoms, or 1 to 6 carbon atoms. Typical alkynylene radicals include, but are not limited to, acetylene (—C≡C—), propargyl (—CH₂C≡C—) and 4-pentynyl (—CH₂CH₂CH₂C≡C—).

“Amino” refers generally to a nitrogen radical which can be considered a derivative of ammonia, having the formula —N(X)₂, where each “X” is independently H, substituted or unsubstituted alkyl, substituted or unsubstituted carbocyclyl, substituted or unsubstituted heterocycle, etc. The hybridization of the nitrogen is approximately sp³. Nonlimiting types of amino include —NH₂, —N(alkyl)₂, —NH(alkyl), —N(cabocylyl)₂, —NH(carbocyclyl), —N(heterocyclyl)₂, —N(heterocyclyl), —N(aryl)₂, —NH(aryl), —N(alkyl)(aryl), —N(alkyl)(heterocyclyl), —N(carbocyclyl)(heterocyclyl), —N(aryl)(heteroaryl), —N(alkyl)(heteroaryl), etc. The term “alkylamino” refers to an amino group substituted with at least one alkyl group. Non-limiting examples of amino groups include —NH₂, —NH(CH₃), —N(CH₃), —N(CH₃)₂, —NH(CH₂CH₃), —N(CH₂CH₃)₂, —NH(phenyl), —N(phenyl)₂, —NH(benzyl), —N(benzyl)₂, etc. Substituted alkylamino refers generally to alkylamino groups, as defined above, in which at least one substituted alkyl, as defined herein, is attached to the amino nitrogen atom. Non-limiting examples of substituted alkylamino includes —NH(alkylene-C(O)—OH), —NH(alkylene-C(O)—O—alkyl), —N(alkylene-C(O)—OH)₂, —N(alkylene-C(O)—O-alkyl)₂, etc.

“Aryl” means an aromatic hydrocarbon radical derived by the removal of one hydrogen atom from a single carbon atom of a parent aromatic ring system. For example, an aryl group can have 6 to 20 carbon atoms, 6 to 14 carbon atoms, or 6 to 10 carbon atoms. Typical aryl groups include, but are not limited to, radicals derived from benzene (e.g., phenyl), substituted benzene, naphthalene, anthracene, biphenyl, and the like.

“Arylalkyl” refers to an acyclic alkyl radical in which one of the hydrogen atoms bonded to a carbon atom, typically a terminal or sp³ carbon atom, is replaced with an aryl radical. Typical arylalkyl groups include, but are not limited to, benzyl, 2-phenylethan-1-yl, naphthylmethyl, 2-naphthylethan-1-yl, naphthobenzyl, 2-naphthophenylethan-1-yl, and the like. The arylalkyl group can comprise 7 to 20 carbon atoms, e.g., the alkyl moity is 1 to 6 carbon atoms and the aryl moiety is 6 to 14 carbon atoms.

“Arylalkenyl” refers to an acyclic alkyl radical in which one of the hydrogen atoms bonded to a carbon atom, typically a terminal or sp³ carbon atom, but also an sp² carbon atom, is replaced with an aryl radical. The aryl portion of the arylakenyl can include, for example, any of the aryl groups disclosed herein, and the alkenyl portion of the arylalkenyl can include, for example, any of the alkenyl groups disclosed herein. The arylalkenyl group can comprise 8 to 20 carbon atoms e.g. the alkenyl moiety is 2 to 6 carbon atoms and the aryl moiety is 6 to 14 carbon atoms.

“Arylalkynyl” refers to an acyclic alkyl radical in which one of the hydrogen atoms bonded to a carbon atom, typically a terminal or sp³ carbon atom, but also an sp carbon atom, is replaced with an aryl radical. The aryl portion of the arylakynyl can include, for example, any of the aryl groups disclosed herein, and the alkynyl portion of the arylalkynyl can include, for example, any of the alkynyl groups disclosed herein. The arylalkynyl group can comprise 8 to 20 carbon atoms e.g. the alkynyl moiety is 2 to 6 carbon atoms and the aryl moiety is 6 to 14 carbon atoms.

The term “substituted” in reference to alkyl, alkene, aryl, arylalkyl, alkoxy, heterocyclyl, heteraryl, carbocyclyl, etc., for example, “substituted alkyl”, “substituted alkylene”, “substituted aryl”, “substituted arylalkyl”, “substituted heterocyclyl”, and “substituted carbocyclyl” means alkyl, alkylene, aryl, arylalkyl, heterocyclyl, carbocyclyl respectivelyl, in which one or more hydrogen atoms are each independently replaced with a non-hydrogen substituent. Typical substituents include, but are not limited to —X, —R^{b}, —O⁻, ═O, —OR^{b}, —SR^{b}, —S⁻, —NR^{b}₂, —N⁺R^{b}₃═NR^{b}, —CX₃, —CN, —OCN, —SCN, —N═C═O, —NCS, —NO, —NO₂, ═N2, N3, —NHC(═O)R^{b}, —OC(═O)R^{b}, —NHC(═O)NR^{b}₂, —S(═O)₂—, —S(═O)₂OH, —S(═O)₂R^{b}, —OS(═O)₂OR^{b}, —OS(═O)₂NR^{b}₂, —S(═O)R^{b}, —OP(═O)(OR^{b})₂, —P(═O)(OR^{b})₂, —P(═O)(O⁻)₂, —P(═O)(OH)₂, —P(═O)(OR^{b})(O⁻), —C(═O)R^{b}, —C(═O)X, —C(S)R^{b}, —C(O)OR^{b}, —C(O)O⁻, —C(S)OR^{b}, —C(O)SR^{b}, —C(S)SR^{b}, —C(O)NR^{b}, —C(S)NR^{b}₂, —C(═NR^{b})NR^{b}₂, where each X is independently a halogen: F, Cl, Br, or I; and each R^{b} is independently H, alkyl, aryl, arylalkyl, a heterocycle, or a protecting group or prodrug moiety. Alkylene, alkenylene, and alkynylene groups may also be similarly substituted. Unless otherwise indicated, when the term “substituted” is used in conjunction with groups such as arylalkyl, which have two or more moieties capable of substitution, the substituents can be attached to the aryl moiety, the alkyl moiety, or both.

A “prodrug” is defined in the pharmaceutical field as a biologically inactive derivative of a drug that upon administration to the human body is converted to the biologically active parent drug according to some chemical or enzymatic pathway (Bundgaard, Hans, “Design and Application of Prodrugs” in Textbook of Drug Design and Development (1991), P. Krosgaard-Larsen and H. Bundgaard, Eds. Harwood Academic Publishers, pp. 113-191). Enzymes which are capable of an enzymatic activation mechanism with the phosphate or phosphonate prodrug compounds of the invention include, but are not limited to, amidases, esterases, microbial enzymes, phospholipases, cholinesterases, and phosphatases. Prodrug moieties can serve to enhance solubility, absorption and lipophilicity to optimize drug delivery, bioavailability and efficacy.

A prodrug moiety may include an active metabolite or drug itself.

Exemplary prodrug moieties include the hydrolytically sensitive or labile acyloxymethyl esters —CH₂₀C(═O)R³⁰ and acyloxymethyl carbonates CH₂OC(═O)O R³⁰ where R³⁰ is C₁-C₆ alkyl, C₁-C₆ substituted alkyl, C₆-C₂₀ aryl, or C₆-C₂₀ substituted aryl. The acyloxyalkyl ester was used as a prodrug strategy for carboxylic acids and then applied to phosphates and phosphonates by Farquhar et al. (1983) J. Pharm. Sci. 72: 324; also U.S. Pat. Nos. 4,816,570, 4,9687,88, 5,663,159 and 5,792,756. In certain compounds of the invention, a prodrug moiety is part of a phosphate or phosphonate group. The acyloxyalkyl ester may be used to deliver phosphates across cell membranes and to enhance oral bioavailability. A close variant of the acyloxyalkyl ester, the alkoxycarbonyl ester (carbonate) may also enhance oral bioavailability as a prodrug moiety in the compounds of the combinations of the invention. An exemplary acyloxymethyl ester is pivaloyloxymethyl (POM) —CH₂OC(═O)C(CH₃)₃. An exemplary acyloxymethyl carbonate prodrug is pivaloyloxymethylcarbonate (POC) —CH₂OC(═O)OCH(CH₃)₂.

The phosphate group may be a phosphate prodrug moiety. The prodrug moiety may be sensitive to hydrolysis, such as, but not limited to those comprising a pivaloyloxymethyl carbonate (POC) or POM group. Alternatively, the prodrug moiety may be sensitive to enzymatic potentiated cleavage, such as a lactate ester or a phosphoramidate-ester group.

Aryl esters of phosphorous groups, especially phenyl esters are reported to enhance oral bioavailability. (DeLambert et al. (1994) J. Med. Chem. 37: 498). Phenyl esters containing a carboxylic ester ortho to the phosphate have also been described (Khamnei and Torrence, (1996) J. Med. Chem. 39:4109-4115). Benzyl esters are reported to generate the parent phosphoric or phosphonic acid. In some cases, substituents at the ortho- or para-position may accelerate the hydrolysis. Benzyl analogues with an acylated phenol or an alkylated phenol may generate the phenolic compound through the action of enzymes e.g., esterases, oxidases, etc., which in turn undergoes cleavage at the benzylic C—O bond to generate the phosphoric or phosphonic acid and the quinone methide intermediate. Examples of this class of prodrugs are described by Mitchell et al. (1992) J. Chem. Soc. Perkin Trans. 12345; Brook et al. WO 91/19721. Still other benzylic prodrugs have been described containing a carboxylic ester-containing group attached to the benzylic methylene (Glazier et al. WO 91/19721). Thiol-containing prodrugs are reported to be useful for the intracellular delivery of phosphate or phosphonate drugs. These pro-esters contain an ethylthiol group in which the thiol group is either esterified with an acyl group or combined with another thiol group to form a disulfide. De-esterification or reduction of the disulfide generates the free thiol intermediate which subsequently breaks down to the phosphoric or phosphonic acid and episulfide (Puech et al. (1993) Antiviral Res., 22:155-174; Benzaria et al. (1996) J. Med. Chem. 39: 4958). Cyclic phosphonate or phosphate esters have also been described as prodrugs of phosphorus-containing compounds (Erion et al. U.S. Pat. No. 6,312,662).

One skilled in the art will recognize that substituents and other moieties of the compounds of Formula I-III should be selected in order to provide a compound which is sufficiently stable to provide a pharmaceutically useful compound which can be formulated into an acceptably stable pharmaceutical composition. Compounds of Formula I-III which have such stability are contemplated as falling within the scope of the present invention.

“Heteroalkyl” refers to an alkyl group where one or more carbon atoms have been replaced with a heteroatom such as O, N, or S. For example, if the carbon atom of the alkyl group which is attached to the parent molecule is replaced with a heteroatom (e.g., O, N, or S), the resulting heteroalkyl groups, are, respectively an alkoxy group (e.g., —OCH₃, etc.), an amine (e.g. —NHCH₃, —N(CH₃)₂, etc.), or a thioalkyl group (e.g., —SCH₃). If a non-terminal carbon atom of the alkyl group which is not attached to the parent molecule is replaced with a heteroatom (e.g., O, N, or S) the resulting heteroalkyl groups are, respectively, an alky ether (e.g., —CH₂CH₂—O—CH₃, etc.), an alkyl amine (e.g., —CH₂NHCH₃, —CH₂N(CH₃)₂, etc.), or a thioalkyl ether (e.g. —CH₂—S—CH₃). If a terminal carbon atom of the alkyl group is replaced with a heteroatom (e.g., O, N, or S), the resulting heteroalkyl groups are, respectively, a hydroxyalkyl group (e.g., —CH₂CH₂—OH), an minoalkyl group (e.g. —CH₂NH₂), or an alkyl thiol group (e.g., —CH₂CH₂—SH). A heteroalkyl group can have, for example, 1 to 20 carbon atoms, 1 to 10 carbon atoms, or 1 to 6 carbon atoms. A C₁-C₆ heteroalkyl group means a heteroalkyl group having 1 to 6 carbon atoms.

“Heterocycle” or “heterocyclyl” as used herein includes by way of example and not limitation those heterocycles described in Paquette, Leo A.; Principles of Modern Heterocyclic Chemistry (W. A. Benjamin, New York, 1968), particularly Chapters 1, 3,4, 6, 7, and 9; The Chemistry of Heterocyclic Compounds, A Series of Monogrpahs (John Wiley & Sons, New York, 1950 to present), in particular Volumes 13, 14, 16, 19, and 28; and J. Am. Chem. Soc. (1960) 82:5566. The terms “heterocycle” or “heterocyclyl” includes saturated rings, particularly unsaturated rings, and aromatic rings (i.e., heteroaromatic rings). Substituted heterocycles include, for example, heterocyclic rings substituted with any of the substituents disclosed herein including carbonyl groups. A non-limiting example of a carbonyl substituted heterocyclyl is:

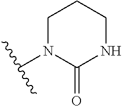 [SMILES: *N1CCCNC1=O]

Examples of heterocycles include by way of example and not limitation pyridyl, dihydropyridyl, tetrahydropyridyl (piperidyl), thiazolyl, tetrahydrothiophenyl, sulfur oxidized tetrahydrothiophenyl, pyrimidinyl, furanyl, thienyl, pyrrolyl, pyrazolyl, imidazolyl, tetrazolyl, benzofuranyl, thianaphthalenyl, indolyl, indolenyl, quinolinyl, isoquinolinyl, benzimidazolyl, piperidinyl, 4-piperidonyl, pyrrolidinyl, 2-pyrrolidonyl, pyrrolinyl, tetrahydrofuranyl, tetrahydroquinolinyl, tetrahydroisoquinolinyl, decahydroquinolinyl, octahydroisoquinolinyl, azocinyl, triazinyl, 6H-1,2,5-thiadiazinyl, 2H, 6H-1,5,2-dithiazinyl, thienyl, thianthrenyl, pyranyl, isobenzofuranyl, chromenyl, xanthenyl, phenoxathinyl, 2H-pyrrolyl, isothiazolyl, isoxazolyl, pyrazinyl, pyridazinyl, indolizinyl, isoindolyl, 3H-indolyl, 1H-indazolyl, purinyl, 4H-quinolizinyl, pthalazinyl, naphthyridinyl, quinoxalinyl, quinazolinyl, cinnolinyl, pteridinyl, 4H-carbazolyl, carbazolyl, β-carbolinyl, phenathridinyl, acridinyl, phenanthrolinyl, phenazinyl, phenothiazinyl, furazanyl, phenoxazinyl, isochromanyl, chromanyl, imidazolidinyl, imidazolinyl, pyrazolidinyl, pyrazolinyl, piperzinyl, indolinyl, isoindolinyl, quinuclidinyl, morpholinyl, oxazolidinyl, benzotriazolyl, benzioxazolyl, oxindolyl, benzoazolinyl, isatinoyl, and bis-tetrahydrofuranyl:

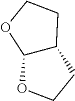

Other non-limiting examples of heterocycles include, for example, dihydropyridyl isomers, piperidine, pyridazinyl, pyrimidinyl, pyrazinyl, s-triazinyl, oxazolyl, thiazolyl, isoxazolyl, pyrazolyl, isothiazolyl, thiofuranyl,

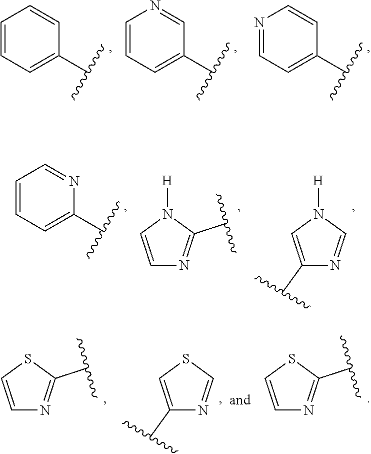

Heterocycles may be independently substituted with 0 to 3 R groups, as defined above.

By way of example and not limitation, carbon bonded heterocycles are bonded at position 2, 3, 4, 5, or 6 of a pyridine position 3, 4, 5, or 6 of a pyridazine, position 2, 4, 5, or 6 of a pyrimidine, position 2, 3, 5, or 6 of a pyrazine, position 2, 3, 4, or 5 of a furan, tetrahydrofuran, thiofuran, thiophene, pyrrole or tetrahydropyrrole, position 2, 4, or 5 of an oxazole, imidazole, or thiazole, position 3, 4, or 5 of an isoxazole, pyrazole, or isothiazole, position 2 or 3 of an aziridine, position 2, 3, or 4 of an azetidine, position 2, 3, 4, 5, 6, 7, or 8 of a quinoline or position 1, 3, 4, 5, 6, 7, or 8 of an isoquinoline. Still more typically, carbon bonded heterocycles include 2-pyridyl, 3-pyridyl, 4-pyridyl, 5-pyridyl, 6-pyridyl, 3-pyridazinyl, 4-pyridazinyl, 5-pyridazinyl, 6-pyridazinyl, 2-pyrimidinyl, 4-pyrimidinyl, 5-pyrimidinyl, 6-pyrimidinyl, 2-pyraizinyl, 3-pyrazinyl, 5-pyrazinyl, 6-pyrazinyl, 2-thiazolyl, 4-thiazolyl, or 5-thiazolyl.

By way of example and not limitation, nitrogen bonded heterocycles are bonded at position 1 of an aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, 1H-indazole, position 2 of an isoindole or isoindoline, position 4 of a morpholine, and position 9 of a carbazole or β-carboline. Still more typically, nitrogen bonded heterocycles include 1-aziridyl, 1-azetedyl, 1-pyrrolyl, 1-imidazolyl, 1-pyrazolyl, and 1-piperidinyl.

“Heterocyclylalkyl” refers to an acyclic alkyl radical in which one of the hydrogen atoms bonded to a carbon atom, typically a terminal or sp³ carbon atom, is replaced with a heterocyclyl radical (i.e., a heterocyclyl-alkylene-moiety). Typical heterocyclyl alkyl groups include, but are not limited to, heterocyclyl-CH₂-2(heterocyclypethan-1-yl, and the like, wherein the “heterocyclyl” portion includes any of the heterocyclyl groups described above, including those described in Principles of Modern Heterocyclic Chemistry. One skilled in the art wil also understand that the heterocyclyl group can be attached to the alkyl portion of the heterocyclyl alkyl by means of a carbon-carbon bond or a carbon-heteroatom bond, with the proviso that the resulting group is chemically stable. The heterocyclyl alkyl group comprises 3 to 20 carbon atoms, e.g., the alkyl portion of the arylalkyl group is 1 to 6 carbon atoms and the heterocyclyl moiety is 2 to 14 carbon atoms. Examples of heterocyclylalkyls include, by way of example and not limitation, 5-membered sulfur-, oxygen-, and/or nitrogen-containing heterocycles such as thiazolylmethyl, 2-thiazolylethan-1-yl, imidazolylmethyl, oxazolylmethyl, thiadiazolylmethyl, etc., 6-membered sulfur-, oxygen-, and/or nitrogen-containing heterocycles such as piperidinylmethyl, piperazinylmethyl, morpholinylmethyl, piperidinylmethyl, pyridinylmethyl, pyridizylmethyl, pyrimidylmethyl, pyrazinylmethyl, etc.

“Heterocyclylalkenyl” refers to an acyclic alkenyl radical in which one of the hydrogen atoms bonded to a carbon atom, typically a terminal or sp³ carbon atom, but also an sp² carbon atom, is replaced with a heterocyclyl radical (i.e., a heterocyclyl-alkenylene-moiety). The heterocyclyl portion of the heterocyclyl alkenyl group includes any of the heterocyclyl groups described herein, including those described in Principles of Modern Heterocyclic Chemistry, and the alkenyl portion of the heterocyclyl alkenyl group includes any of the alkenyl groups disclosed herein. One skilled in the art will also understand that the heterocyclyl group can be attached to the alkenyl portion of the heterocyclyl alkenyl by means of a carbon-carbon bond or a carbon-heteroatom bond, with the proviso that the resulting group is chemically stable. The heterocyclyl alkenyl group comprises of 4 to 20 carbon atoms, e.g., the alkenyl portion of the heterocyclyl alkenyl group is 2 to 6 carbon atoms and the heterocyclyl moiety is 2 to 14 carbon atoms.

“Heterocyclylalkynyl” refers to an acyclic alkenyl radical in which one of the hydrogen atoms bonded to a carbon atom, typically a terminal or sp³ carbon atom, but also an sp carbon atom, is replaced with a heterocyclyl radical (i.e., a heterocyclyl-alkynylene-moiety). The heterocyclyl portion of the heterocyclyl alkynyl group includes any of the heterocyclyl groups described herein, including those described in Principles of Modern Heterocyclic Chemistry, and the alkynyl portion of the heterocyclyl alkynyl group includes any of the alkynyl groups disclosed herein. One skilled in the art will also understand that the heterocyclyl can be attached to alkynyl portion of the heterocyclyl alkynyl by means of a carbon-carbon bond or a carbon-heteroatom bond, with the proviso that the resulting group is chemically stable. The heterocyclyl alkynyl group comprises 4 to 20 carbon atoms, e.g., the alkynyl portion of the heterocyclyl alkynyl group is 2 to 6 carbon atoms and the heterocyclyl moiety is 2 to 14 carbon atoms.

“Heteroaryl” refers to an aromatic heterocyclyl having at least one heteroatom in the ring. Non-limiting examples of suitable heteroatoms which can be included in the aromatic ring include oxygen, sulfur, and nitrogen. Non-limiting examples of heteraryl rings include all of those aromatic rings listed in the definition of “heterocyclyl”, including pyridinyl, pyrrolyl, oxazolyl, indolyl, isoindolyl, purinyl, furanyl, thienyl, benzofuranyl, benzothiophenyl, carbazoylyl, imidazolyl, thiazolyl, isoxazolyl, pyrazolyl, isothiazolyl, quinolyl, isoquinolyl, pyradazyl, pyrimidyl, pyrazyl, etc.

“Carbocycle” or “carbocyclyl” refers to a saturated (i.e., cycloalkyl), partially unsaturated (e.g., cycloalkenyl, cycloalkadienyl, etc.) or aromatic ring having 3 to 7 carbon atoms as a monocycle, 7 to 12 carbon atoms as a bicycle, and up to about 20 carbon atoms as a polycycle. Monocyclic carbocycles have 3 to 7 ring atoms, still more typically 5 or 6 ring atoms. Bicyclic carbocycles have 7 to 12 ring atoms, e.g., arranged as a bicyclo [4,5], [5,5], [5,6] or [6,6] system, or 9 or 10 ring atoms arranged as a bicyclo [5,6] or [6,6] system, or spiro-fused rings. Non-limiting examples of monocyclic carbocycles include cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, and phenyl. Non-limiting examples of bicyclo carbocycles include naphthyl, tetrahydronaphthalene, and decaline. Carbocycles may be independently substituted with 0 to 3 R groups, as defined above. Non-limiting examples of carbocycles include:

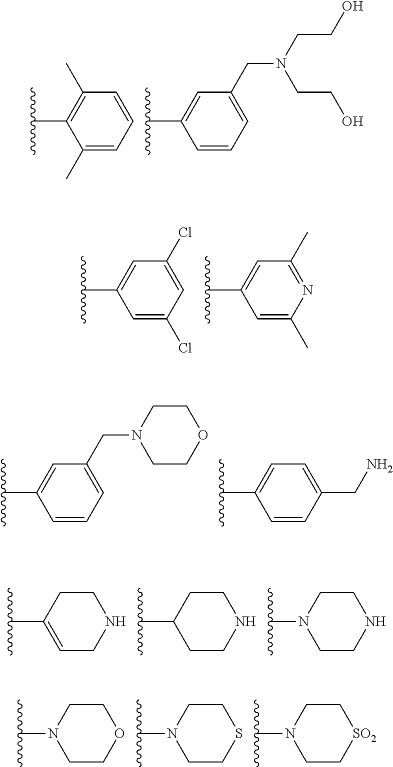

Examples of substituted phenyl carbocycles include:

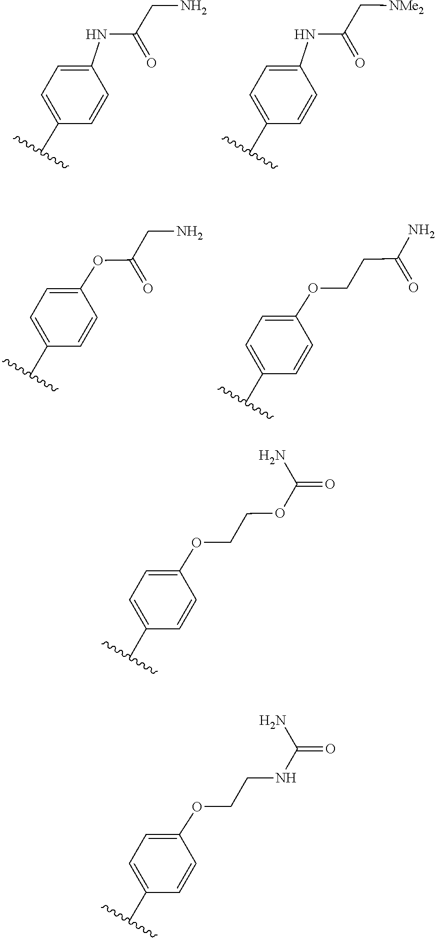 [SMILES: *c1ccc(NC(=O)CN)cc1 ; *c1ccc(NC(=O)CN(C)C)cc1 ; *c1ccc(OC(=O)CN)cc1 ; *c1ccc(OCCC(N)=O)cc1 ; *c1ccc(OCCOC(N)=O)cc1 ; *c1ccc(OCCNC(N)=O)cc1]

“Carbocyclylalkyl” refers to an acyclic alkyl radical in which one of the hydrogen atoms bonded to a carbon atom is replaced with a carbocyclyl radical as described herein. Typical, but non-limiting examples of carbocyclylalkyl group sinclude cyclopropylmethyl, cyclopropylethyl, cyclobutylmethyl, cyclopentylmethyl, and cyclohexylmethyl.

“Arylheteroalkyl” refers to a heteroalkyl as defined herein, in which a hydrogen atom (which may be attached either to a carbon atom or a heteroatom) has been replaced with an aryl group as defined herein. The aryl groups may be bonded to a carbon atom of the heteroalkyl group, or to a heteroatom of the heteroalkyl group, provided that the resulting arylheteroalkyl group provides a chemically stable moiety. For example, an arylheteroalkyl group can have the general formulae -alkylene-O-aryl, -alkylene-O-alkylene-aryl, -alkylene-NH-aryl, -alkylene-NH-alkylene-aryl, -alkylene-S-aryl, -alkylene-S-alkylene-aryl, etc. In addition, any of the alkylene moieties in the general formulae above can be further substituted with any of the substituents defined or exemplified herein.

“Heteroarylalkyl” refers to an alkyl group, as defined herein, in which a hydrogen atom has been replaced with a heteroaryl group as defined herein. Non-limiting example sof heteroaryl alkyl include —CH₂-pyridinyl, —CH₂-pyrrolyl, —CH₂-oxazolyl, —CH₂-indolyl, —CH₂-isoindolyl, —CH₂-purinyl, —CH₂-furanyl, —CH₂-thienyl, —CH₂-benzofuranyl, —CH₂-benzothiophenyl, —CH₂-carbazolyl, —CH₂-imidazolyl, —CH₂-thiazolyl, —CH₂-isoxazolyl, —CH₂-pyrazolyl, —CH₂-isothiazolyl, —CH₂-quinolyl, —CH₂-isoquinolyl, —CH₂-pyridazyl, —CH₂-pyrimidyl, —CH₂-pyrazyl, —CH(CH₃)-pyridinyl, —CH(CH₃)-pyrrolyl, —CH(CH₃)-oxazolyl, —CH(CH₃)-indolyl, —CH(CH₃)-isoindolyl, —CH(CH₃)-purinyl, —CH(CH₃)-furanyl, —CH(CH₃)-thienyl, —CH(CH₃)-benzofuranyl, —CH(CH₃)-benzothiophenyl, —CH(CH₃)-carbazolyl, —CH(CH₃)-imidazolyl, —CH(CH₃)-thiazolyl, —CH(CH₃)-isoxazolyl, —CH(CH₃)-pyrazolyl, —CH(CH₃)-isothiazolyl, —CH(CH₃)-quinolyl, —CH(CH₃)-isoquinolyl, —CH(CH₃)-pyridazyl, —CH(CH₃)-pyrimidyl, —CH(CH₃)-pyrazyl, etc.

The term “optionally substituted” in reference to a particular moiety of the compound of Formula I-III (e.g., an optionally substituted aryl group) refers to a moiety wherein all substituents are hydrogen or wherein one or more of the hydrogens of the moiety may be replaced by substituents such as those listed under the definition of “substituted”.

The term “optionally replaced” in reference to a particular moiety of the compound of Formula I-III (e.g., the carbon atoms of said (C₁-C₈)alkyl may be optionally replaced by —O—, —S—, or —NR^{a}—) means that one or more of the methylene groups of the (C₁-C₈)alkyl may be replaced by 0, 1, 2, or more of the groups specified (e.g., —O—, —S—, or —NR^{a}—).

The term “non-terminal carbon atom(s)” in reference of an alkyl, alkenyl, alkynyl, alkylene, alkenylene, or alkynylene moiety refers to the carbon atoms in the moiety that intervene between the first carbon atom of the moiety and the last carbon atom in the moiety. Therefore, by way of example and not limitation, in the alkyl moiety —CH₂(C*)H₂(C*)H₂CH₃ or the alkylene moiety —CH₂(C*)H₂CH₂— the C* atoms would be considered to be the non-terminal carbon atoms.

Certain Q and Q¹ alternatives are nitrogen oxides such as ⁺N(O)(R) or ⁺N(O)(OR). These nitrogen oxides, as shown here attached to a carbon atom, can also be represented by charge separated groups such as

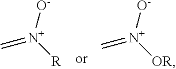

respectively, and are intended to be equivalent to the aforementioned representations for the purposes of describing this invention.

“Linker” or “link” means a chemical moiety comprising a covalent bond or a chain of atoms. Linkers include repeating units of alkyloxy (e.g. polyethyleneoxy, PEG, polymethyleneoxy) and alkylamino (e.g. polyethyleneamino, Jeffamine™); and diacid ester and amides including succinate, succinimide, diglycolate, malonate, and caproamide.

The terms such as “oxygen-linked”, “nitrogen-linked”, “carbon-linked”, “sulfur-linked”, or “phosphorous-linked” mean that if a bond between two moieties can be formed by using more than one type of atom in a moiety, then the bond formed between the moieties is through the atom specified. For example, a nitrogen-linked amino acid would be bonded through a nitrogen atom of the amino acid rather than through an oxygen or carbon atom of the amino acid.

In some embodiments of the compounds of Formulas I-III, one or more Z¹ or Z² are independently a radical of a nitrogen-linked naturally occurring α-amino acid ester. Examples of naturally occurring amino acids include isoleucine, leucine, lysine, methionine, phenylalanine, threonine, tryptophan, valine, alanine, asparagine, aspartic acid, cysteine, glutamic acid, glutamine, glycine, proline, selenocysteine, serine, tyrosine, arginine, histidine, ornithine and taurine. The esters of these amino acids comprise any of those described for the substituent R, particularly those in which R is optionally substituted. (C₁-C₈) alkyl.

The term “purine” or “pyrimidine” base comprises, but is not limited to, adenine, N⁶-alkylpurines, N⁶-acylpurines (wherein acyl is C(O)(alkyl, aryl, alkylaryl, or arylalkyl), N⁶-benzylpurine, N⁶-halopurine, N⁶-vinylpurine, N⁶-acetylenic purine, N⁶-acyl purine, N⁶-hydroxyalkyl purine, N⁶-allylaminopurine, N⁶-thioallyl purine, N²-alkyl purine, N²-alkyl-6-thiopurine, thymine, cytosine, 5-fluorocytosine, 5-methylcytosine, 6-azapyrimidine, including 6-azacytosine, 2- and/or 4-mercaptopyrimidine, uracil, 5-halouracil, including 5-fluorouracil, C⁵-alkylpyrimidines, C⁵-benzylpyrimidines, C⁵-halopyrimidines, C⁵-vinylpyrimidine, C⁵-acetylenic pyrimidine, C⁵-iodopyrimidine, C⁶-iodo-pyrimidine, C⁵—Br-vinyl pyrimidine, C⁵—Br-vinyl pyriniidine, C⁵-nitropyrimidine, C⁵-amino-pyrimidine, N²-alkylpurines, N²-alkyl-6-thiopurines, 5-azacytidinyl, 5-azauracilyl, triazolopyridinyl, imidazolopyridinyl, pyrrolopyrimidinyl, and pyrazolopyrimidinyl. Purine bases include, but are not limited to, guanine, adenine, hypoxanthine, 2,6-diaminopurine, and 6-chloropurine. The purine and pyrimidine bases of Formulas I-III are linked to a ribose sugar, or analogue thereof, through a nitrogen or carbon atom of the base. Functional oxygen and nitrogen groups on the base can be protected as necessary or desired. Suitable protecting groups are well-known to those skilled in the art, and include trimethylsilyl, dimethylhexylsilyl, t-butyldimethylsilyl, and t-butyldiphenylsilyl, trityl, alkyl groups, and acyl groups such as acetyl, and propionyl, methanesulfonyl, and p-toluenesulfonyl.

Unless otherwise specified, the carbon atoms of the compounds of Formulas I-III are intended to have a valence of four. In some chemical structure representations where carbon atoms do not have a sufficient number of variables attached to produce a valence of four, the remaining carbon substituents need to provide a valence of four should be assumed to be hydrogen. For example,

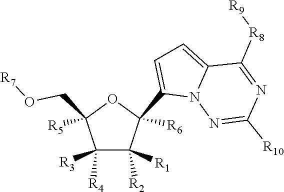

has the same meaning as

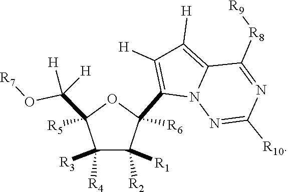

“Protecting group” refers to a moiety of a compound that masks or alters the properties of a functional group or the properties of the compound as a whole. The chemical substructure of a protecting group varies widely. One function of a protecting group is to serve as an intermediate in the synthesis of the parental drug substance. Chemical protecting groups and strategies for protection/deprotection are well-known in the art. See: “Protective Groups in Organic Chemistry”, Theodora W. Greene (John Wiley & Sons, Inc., New York, 1991). Protecting groups are often used to mask the reactivity of certain functional groups, to assist in the efficiency of desired chemical reactions, e.g., making and breaking chemical bonds in an ordered and planned fashion. Protection of functional groups of a compound alters other physical properties besides the reactivity of the protected functional group, such as the polarity, lipophilicity (hydrophobicity), and other properties which can be measured by common analytical tools. Chemically protected intermediates may themselves be biologically active or inactive. “Hydroxy protecting groups” refers to those protecting useful for protecting hydroxy groups (—OH).

Protected compounds may also exhibit altered, and in some cases, optimized properties in vitro and in vivo, such as passage through cellular membranes and resistance of enzymatic degradation or sequestration. In this role, protected compounds with intended therapeutic effects may be referred to as prodrugs. Another function of a protecting group is to convert the parental drug into a prodrug, whereby the parental drug is released upon conversion of the prodrug in vivo. Because active prodrugs may be absorbed more effectively than the parental drug, prodrugs may possess greater potency in vivo than the parental drug. Protecting groups are removed either in vitro, in the instance of chemical intermediates, or in vivo, in the case of prodrugs. With chemical intermediates, it is not particularly important that the resulting products after deprotection, e.g., alcohols, be physiologically acceptable, although in general it is more desirable if the products are pharmacologically innocuous.

The term “chiral” refers to molecules which have the property of non-superimposability of the mirror image partner, while the term “achiral” refers to the molecules which are superimposable on their mirror image partner.

The term “stereoisomers” refers to compounds which have identical chemical constitution but differ with regard to the arrangement of the atoms or groups in space.

“Diastereomer” refers to a stereoisomer with two or more centers of chirality and whose molecules are not mirror images of one another. Diastereomers have different physical properties, e.g., melting points, boiling points, spectral properties, reactivities and biological properties. For example, the compounds of Formulas I-III may have a chiral phosphorous atom when R⁷ is

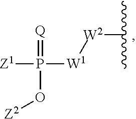

and Z¹ and Z² are different. When at least one of either Z¹ or Z² also has a chiral center, for example with Z¹ or Z² is a nitrogen-linked chiral, naturally occurring α-amino acid ester, then the compound of Formulas I-III will exist as diastereomers because there are two centers of chirality in the molecule. All such diastereomers and their uses described herein are encompassed by the instant invention. Mixtures of diastereomers may be separate under high resolution analytical procedures such as electrophoresis, crystallization and/or chromatography. Diastereomers may have different physical attributes such as, but not limited to, solubility, chemical stabilities and crystallinity and may also have different biological properties such as, but not limited to, enzymatic stability, absorption and metabolic stability.

“Enantiomers” refer to two stereoisomers of a compound which are non-superimposable mirror images of one another.

The modifier “about” used in connection with a quantity is inclusive of the stated value and has the meaning dictated by the context (e.g., includes the degree of error associated with measurement of the particular quantity).

The term “treating”, as used herein, unless otherwise indicated, means reversing, alleviating, inhibiting the progress of, or preventing the disorder or condition to which such term applies, or one or more symptoms of such disorder or condition. The term “treatment”, as used herein, refers to the act of treating, as “treating” is defined immediately above.

The term “therapeutically effective amount”, as used herein, is the amount of compound of Formula I-III present in a composition described herein that is needed to provide a desired level of drug in the secretions and tissues of the airways and lungs, or alternatively, in the bloodstream of a subject to be treated to give an anticipated physiological response or desired biological effect when such a composition is administered by the chosen route of administration. The precise amount will depend on numerous factors, for example, the particular compound of Formula I-III, the specific activity of the composition, the delivery device employed, the physical characteristics of the composition, its intended use, as well as patient considerations such as severity of the disease state, patient cooperation, etc., and can readily be determined by one skilled in the art based upon the information provided herein.

The term “normal saline” means a water containing 0.9% (w/v) NaCl.

The term “hypertonic saline” means a water solution containing greater than 0.9% (w/v) NaCl. For example, 3% hypertonic saline would contain 3% (w/v) NaCl.

“Forming a reaction mixture” refers to the process of bringing into contact at least two distinct species such that they mix together and can react. It should be appreciated however, the resulting reaction product can be produced directly from a reaction between the added reagents or from an intermediate from one or more of the added reagents which can be produced in the reaction mixture.

“Coupling agent” refers to an agent capable of coupling tow disparate compounds. Coupling agents can be catalytic or stoichiometric. For example, coupling agents can be a lithium-based coupling agent or a magnesium-based coupling agent such as a Grignard reagent. Exemplary coupling agents include, but are not limited to, n-BuLi, MgCl₂, iPrMgCl, tBuMgCl, PhMgCl or combinations thereof.

“Silane” refers to a silicon-containing group having the formula SiR₄, where each R group can be alkyl, alkenyl, cycloalkyl, phenyl, or other silicon-containing groups. When the silane is linked to another compound, the silane is referred to as “silyl” and has the formula —SiR₃—.

“Halo-silane” refers to a silane having at least one halogen group linked to the silicon atom. Representative hal-silanes have the formula Halo-SiR₃, where each R group can be alkyl, alkenyl, cycloalkyl, phenyl, or other silicon-containing groups. Specific halo-silanes include Cl—Si(CH₃)₃, and Cl—Si(CH₃)₂CH₂CH₂Si(CH₃)₂—Cl.

“Non-nucleophilic base” refers to an electron donor, a Lewis base, such as nitrogen bases including triethylamine, diisopropylethylamine, N,N-diethylaniline, pyridine, 2,6-lutidine, 2,4,6-collidine, 4-dimethylaminopyridine, and quinuclidine.

“Leaving group” refers to groups that maintain the bonding electron pair during heterocyclic bond cleavage. For example, a leaving group is readily displaced during a nucleophilic displacement reaction. Suitable leaving groups include, but are not limited to, chloride, bromide, mesylate, tosylates, triflate, 4-nitrobenzenesulfonate, 4-chlorobenzenesulfonate, 4-nitrophenoxy, pentafluorophenoxy, etc. One skilled in the art will recognize other leaving groups useful in the present invention.

“Deprotection agent” refers to any agent capable of removing a protecting group. The deprotection agent will depend on the type of protecting group used. Representative deprotection agents are known in the art and can be found in Protective Groups in Organic Chemistry, Peter G. M. Wuts and Theodora W. Greene, 4^{th} Ed., 2006.

It is to be noted that all enantiomers, diastereomers, and racemic mixtures, tautomers, polymorphs, pseudopolymorphs of compounds within the scope of Formulas I-III and pharmaceutically acceptable salts thereof are embraced by the present invention. All mixtures of such enantiomers and diastereomers are within the scope of the present invention.

A compound of Formula I-III and its pharmaceutically acceptable salts may exist as different polymorphs or pseudopolymorphs. As used herein, crystalline polymorphism means the ability of a crystalline compound to exist in different crystal structures. The crystalline polymorphism may result from differences in crystal packing (packing polymorphism) or differences in packing between different conformers of the same molecule (conformational polymorphism). As used herein, crystalline pseudopolymorphism means the ability of a hydrate or solvate of a compound to exist in different crystal structures. The pseudopolymorphs of the instant invention may exist due to differences in crystal packing (packing pseudopolymorphism) or due to differences in packing between different conformers of the same molecule (conformational pseudopolymorphism). The instant invention comprises all polymorphs and pseudopolymorphs of the compounds of Formulas I-III and their pharmaceutically acceptable salts.

A compound of Formula I-III and its pharmaceutically acceptable salts may also exist as an amorphous solid. As used herein, an amorphous solid is a solid in which there is no long-range order of the positions of the atoms in the solid. This definition applies as well when the crystal size is two nanometers or less. Additives, including solvents, may be used to create the amorphous forms of the instant invention. The instant invention comprises all amorphous forms of the compounds of Formulas I-III and their pharmaceutically acceptable salts.

### III. METHODS OF INHIBITION OF SARS-COV-2 POLYMERASE

Another aspect of the invention relates to methods of inhibiting activity of the SARS-CoV-2 RNA-dependent RNA-polymerase (RdRp) comprising the step of treating a sample suspected of containing SARS-CoV-2 with a composition of the invention.

Compositions of the invention may act as inhibitors of SARS-CoV-2 RdRp, as intermediates for such inhibitors or have other utilities described below. The inhibitors will bind to locations on the surface or in a cavity of the RdRp having a geometry unique to SARS-CoV-2 RdRp. Compositions binding SARS-CoV-2 RdRp may bind with varying degrees of reversibility. Those compounds binding substantially irreversibly are ideal candidates for tuse in this method of the invention. Once labeled, the substantially irreversibly binding compositions are useful as probes for the detection of SARS-CoV-2 polymerase. Accordingly, the invention relates to methods of detecting SARS-CoV-2 polymerase in a sample suspected of containing SARS-CoV-2 polymerase comprising the steps of: treating a sample suspected of containing SARS-CoV-2 polymerase with a composition comprising a compound of the invention bound to a label; and observing the effect of the sample on the activity of the label. Suitable labels are well-known in the diagnostics field and include stable free radicals, fluorophores, radioisotopes, enzymes, chemiluminescent groups and chromogens. The compounds herein are labeled in conventional fashion using functional groups such as hydroxyl, carboxyl, sulfhydryl, or amino.

Within the context of the invention, samples suspected of containing SARS-CoV-2 polymerase include natural or man-made materials such as living organisms; tissue or cell cultures; biological samples such as biological material samples (blood, serum, urine, cereberospinal fluid, tears, sputum, saliva, tissue samples, and the like); laboratory samples; food, water, or air samples; bioproduct samples such as extracts of cells, particularly recombinant cells synthesizing a desired glycoprotein; and the like. Typically, the sample will be suspected of containing an organism which produces SARS-CoV-2 polymerase, frequently a pathogenic organism such as SARS-CoV-2. Samples can be contained in any medium including water and organic solvent\water mixtures. Samples include living organism such as humans, and man-made materials such as cell cultures.

The treating step of the invention comprises adding the composition of the invention to the sample or it comprises adding a precursor of the composition to the sample. The addition step comprises any method of administration as described above.

If desired, the activity of SARS-CoV-2 polymerase after application of the composition can be observed by any method including direct and indirect method for detecting SARS-CoV-2 polymerase activity. Quantitative, qualitative and semiquantitative methods of determining SARS-CoV-2 polymerase activity are all contemplated. Typically, one of the screening methods described above are applied, however, any other method such as observation of the physiological properties of a living organism are also applicable.

Organisms that contain SARS-CoV-2 polymerase include SARS-CoV-2 virus. The compounds of this invention are useful in the treatment or prophylaxis of SARS-CoV-2 infections in animals or in man.

### IV. SCREENS FOR SARS-COV-2 POLYMERASE INHIBITORS

Compositions of the invention are screened for inhibitory activity against SARS-CoV-2 polymerase by any of the conventional techniques for evaluating enzyme activity. Within the context of this invention, typically compositions are first screened for inhibition of SARS-CoV-2 polymerase in vitro and compositions showing inhibitory activity are then screened for activity in vivo. Compositions having in vitro Ki (inhibitory constants) of less than about 5×10⁻⁶ M, typically less than about 1×10⁻⁷ M and preferably less than about 5×10⁻⁸ M are preferred for in vivo use.

### V. PHARMACEUTICAL FORMULATIONS

The compounds of this invention are formulated with conventional carriers and excipients, which will be selected in accord with ordinary practice. Tablets will contain excipients, glidants, fillers, binders, and the like. Aqueous formulations are prepared in sterile form, and when intended for delivery by a route other than oral administration will generally be isotonic. All formulations will optionally contain excipients such as those set forth in the “Handbook of Pharmaceutical Excipients” (1986). Excipients include ascorbic acid and other antioxidants, chelating agents such as EDTA, carbohydrates such as dextran, hydroxyalkylcellulose, hydroxyalkylmethylcellulose, stearic acid and the like. The pH of the formulations ranges from about 3 to 11, but is ordinarily about 7 to 10.

While it is possible for the active ingredients to be administered alone, it may be preferable to present them as pharmaceutical formulations. The formulations, both for veterinary and for human use, of the invention comprise at least one active ingredient, as defined above, together with one or more acceptable carriers thereof and optionally other therapeutic ingredients. The carrier(s) must be “acceptable” in the sense of being compatible with the other ingredients of the formulation and physiologically innocuous to the recipient thereof.

The formulations include those suitable for the forgoing administration routes. The formulations may conveniently be presented in unit dosage form and may be prepared by any of the methods well-known in the art of pharmacy. Techniques and formulations are found in Remington's Pharmaceutical Sciences (Mack Publishing Co., Easton, Pa.). Such methods include the step of bringing into association the active ingredient with the carrier which constitutes one or more accessory ingredients. In general, the formulations are prepared by uniformly and intimately bringing into association the active ingredient with liquid carriers or finely divided solid carriers or both, and then, if necessary, shaping the product.

Formulations of the present invention suitable for oral administration may be presented as discrete units such as capsules, cachets, or tablets each containing a predetermined amount of active ingredient; as a powder or granules; as a solution or suspension in an aqueous or non-aqueous liquid; or as an oil-in-water liquid emulsion or a water-in-oil liquid emulsion. The active ingredient may also be administered as a bolus, electuary, or paste.

A tablet is made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, preservative, surface active or dispersing agent. Molded tablets may be made by molding in a suitable machine a mixture of the powdered active ingredient moistened with an inert liquid diluent. The tablets may optionally be coated or scored and optionally are formulated so as to provide slow or controlled release of the active ingredient therefrom.

For infections of the eye or other external tissues e.g. mouth and skin, the formulations are preferably applied as a topical ointment or cream containing the active ingredients in an amount of, for example, 0.075% to 20% w/w (including active ingredient(s)) in a range between 0.01% and 20% in increments of 0.1% and 20% in increments of 0.1% 2/2 such as 0.6% w/w, 0.7% w/w, etc.), preferably 0.2 to 15% w/w and most preferably 0.5 to 10% w/w. When formulated in an ointment, the active ingredients may be employed with either a paraffinic or water-miscible ointment base. Alternatively, the active ingredients may be formulated in a cream with an oil-in-water cream base.

If desired, the aqueous phase of the cream base may include, for example, at least 30% w/w of a polyhydric alcohol, i.e. an alcohol having two or more hydroxyl groups such as propylene glycol, butane 1,3-diol, mannitol, sorbitol, glycerol and polyethylene glycol (including PEG 400) and mixtures thereof. The topical formulations may desirably include a compound which enhances absorption or penetration of the active ingredient through the skin or other affected areas. Examples of such dermal penetration enhancers include dimethyl sulfoxide and related analogues.

Emulgents and emulsion stabilizers suitable for use in the formulation of the intervention include Tween® 60, Span® 80, cetostearyl alcohol, benzyl alcohol, myristyl alcohol, glyceryl mono-stearate and sodium lauryl sulfate.

The choice of suitable oils or fats for the formulation is based on achieving the desired cosmetic properties. The cream should preferably be a non-greasy, non-staining and washable product with suitable consistency to avoid leakage from tubes or other containers. Straight or branched chain, mono- or dibasic alkyl esters such as diisoadipate, isocetyl stearate, propylene glycol diester of coconut fatty acids, isopropyl myristate, decyl oleate, isopropyl palmitate, butyl stearate, 2-ethylhexyl palmitate, or a blend of branched chain esters known as Cromadol CAP may be used, the last three being preferred esters. These may be used alone or in combination depending on the properties required. Alternatively, high melting point lipids such as white soft paraffin and/or liquid paraffin or other mineral oils are used.

Pharmaceutical formulations according to the present invention comprise a combination according to the invention together with one or more pharmaceutically acceptable carriers or excipients and optionally other therapeutic agents. Pharmaceutical formulations containing the active ingredient may be in any form suitable for the intended method of administration. When used for oral use for example, tablets, troches, lozenges, aqueous or oil suspensions, dispersible powders or granules, emulsions, hard or soft capsules, syrups or elixirs may be prepared. Compositions intended for oral use may be prepared according to any method known to the art for the manufacture of pharmaceutical compositions and such compositions may contain one or more agents including sweetening agents, flavoring agents, coloring agents and preserving agents, in order to provide a palatable preparation. Tablets containing the active ingredient in admixture with non-toxic pharmaceutically acceptable excipient which are suitable for manufacture of tablets are acceptable. These excipients may be, for example, inert diluents such as calcium or sodium carbonate, lactose, calcium or sodium phosphate; granulating and disintegrating agents such as maize starch, or alginic acid; binding agents, such as starch, gelatin or acacia; and lubricating agents, such as magnesium stearate, stearic acid, or talc. Tablets may be uncoated or may ne coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

Formulations for oral use may also be presented as hard gelatin capsules where the active ingredient is mixed with an inert solid diluent, for example calcium phosphate or kaolin, or as soft gelatin capsules wherein the active ingredient is mixed with water or an oil medium, such as peanut oil, liquid paraffin, or olive oil.

Aqueous suspensions of the invention contain the active materials in admixture with excipients suitable for the manufacture of aqueous suspensions. Such excipients include a suspending agent, such as sodium carboxymethylcellulose, methylcellulose, hydroxypropyl methylcellulose, sodium alginate, polyvinylpyrrolidone, gum tragacanth and gum acacia, and dispersing or wetting agents such as a naturally occurring phosphatide (e.g., lecithin), a condensation product of alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyethylene sorbitan monooleate). The aqueous suspension may also contain one or more preservatives such as ethyl or n-propyl p-hydroxy-benzoate, or one or more coloring agents, one or more flavoring agents and one or more sweetening agents, such as sucrose or saccharin.

Oil suspensions may be formulated by suspending the active ingredient in a vegetable oil, such as arachis oil, olive oil, sesame oil, or coconut oil, or in a mineral oil such as liquid paraffin. The oral suspensions may contain a thickening agent, such as beeswax, hard paraffin, or cetyl alcohol. Sweetening agents, such as those set forth above, and flavoring agents may be added to provide a palatable oral preparation. These compositions may be preserved by the addition of an antioxidant such as ascorbic acid.

Dispersible powders and granules of the intervention suitable for preparation of an aqueous suspension by the addition of water provide the active ingredient in admixture with a dispersing or wetting agent, a suspending agent, and one or more preservatives. Suitable dispersing or wetting agents and suspending agents are exemplified by those disclosed above. Additional excipients, for example sweetening, flavoring and coloring agents may also be present.

The pharmaceutical compositions of the invention may also be in the form of oil-in-water emulsions. The oily phase may eb a vegetable oil, such as olive oil or arachis oil, a mineral oil such as liquid paraffin, or mixture of these. Suitable emulsifying agents include naturally occurring gums, such as gum acacia and gum tragacanth, naturally occurring phosphatides such as soybean lecithin, esters or partial esters derived from fatty acids and hexitol anhydrides, such as sorbitan monooleate, and condensation products of these partial esters with ethylene oxide, such as polyoxyethylene sorbitan monooleate. The emulsion may also contain sweetening and flavoring agents. Syrups and elixirs may be formulated with sweetening agents, such as glycerol, sorbitol, or sucrose. Such formulations may also contain a demulcent, a preservative, a flavoring or coloring agent.

The pharmaceutical compositions of the invention may be in the form of a sterile injectable preparation, such as a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parentally acceptable diluent or solvent such as a solution in 1,3-butanediol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose, any bland fixed oil may be employed including synthetic mono- or diglycerides. In addition, fatty acids such as oleic acid may likewise be used in the preparation of injectables.

The amount of active ingredients that may be combined with the carrier material to produce a single dosage form will vary depending upon the host treated and the particular mode of administration. For example, a time-release formulation intended for oral administration to humans may contain approximately 1 to 1000 mg of active material with an appropriate and convenient carrier material which may vary from about 5% to 95% of the total compositions (weight: weight). The pharmaceutical composition can be prepared to provide easily measurable amounts for administration. For example, an aqueous solution intended for intravenous infusion may contain from about 3 to 500 μg of the active ingredient per millimeter of solution to ensure that an infusion of a suitable volume at a rate of about 30 mL/hr can occur

Formulations suitable for topical administration to the eye also include eye drops wherein the active ingredient is dissolved or suspended in a suitable carrier, especially an aqueous solvent for the active ingredient. The active ingredient is preferably present in such formulations in a concentration of 0.5 to 20%, advantageously 0.5 to 10% and particularly about 1.5% w/w.

Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Formulations for rectal administration may be presented as a suppository with a suitable base comprising, for example, cocoa butter or a salicylate.

Formulations suitable for intrapulmonary or nasal administration have a particle size for example in the range of 0.1 to 500 microns, such as 0.5, 1, 30, 35, etc., which is administered by rapid inhalation through the nasal passage or by inhalation through the mouth so as to reach the alveolar sacs. Suitable formulations include aqueous or oily solutions of the active ingredient. Formulations suitable for aerosol or dry powder administration may be prepared according to conventional methods and may be delivered with other therapeutic agents such as compounds heretofore used in the treatment or prophylaxis of SARS-CoV-2 infections as described below.

Formulations suitable for vaginal administration may be presented as pessaries, tampons, creams, gels, pastes, foams, or spray formulations containing in addition to the active ingredient such carriers as are known in the art to be appropriate.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

The formulations are presented in unit-dose or multi-dose containers, for example sealed ampoules and vials, and may be stored in a freeze-dried (lyophilized) condition requiring only the addition of the sterile liquid carrier, for example water for injection, immediately prior to use. Extemporaneous injection solutions and suspensions are prepared from sterile powders, granules and tablets of the kind previously described. Preferred unit dosage formulations are those containing a daily dose or unit daily sub-dose, as herein above recited, or an appropriate fraction thereof, of the active ingredient.

It should be understood that in addition to the ingredients particularly mentioned above the formulations of this invention may include other agents conventional in the art having regard to the type of formulation in question, for example those suitable for oral administration may include flavoring agents.

The invention further provides veterinary compositions comprising at least one active ingredient as above defined together with a veterinary carrier thereof. Veterinary carriers are materials useful for the purpose of administering the composition and may be solid, liquid or gaseous materials which are otherwise inert or acceptable in the veterinary art and are compatible with the active ingredient. These veterinary compositions may be administered orally, parenterally or by any other desired route.

Compounds of the invention are used to provide controlled release pharmaceutical formulations containing as active ingredient one or more compounds of the invention (“controlled release formulations”) in which the release of the active ingredient are controlled and regulated to allow less frequency dosing or to improve the pharmacokinetic or toxicity profile of a given active ingredient.

Effective dose of active ingredient depends at least on the nature of the condition being treated, toxicity, whether the compound is being used prophylactically (lower doses) or against an active viral infection, the method of delivery, and the pharmaceutical formulation, and will be determined by the clinician using conventional dose escalation studies. It can be expected to be from about 0.0001 to about 100 mg/kg body weight per day; typically, from about 0.01 to about 10 mg/kg body weight per day; more typically from about 0.05 to about 0.5 mg/kg body weight per day. For example, the daily candidate dose for an adult human of approximately 70 kg body weight will range from 1 mg to 1000 mg, preferably between 5 mg and 500 mg, and may take the form of single or multiple doses.

### VI. ROUTES OF ADMINISTRATION

One or more compounds of the invention (herein referred to as the active ingredients) are administered by any route appropriate to the condition being treated. Suitable routes include oral, rectal, nasal, topical (including buccal and sublingual), vaginal and parenteral (including subcutaneous, intramuscular, intravenous, intradermal, intrathecal and epidural), and the like. It will be appreciated that the preferred route may vary with for example the condition of the recipient. An advantage of the compounds of this invention is that they are orally bioavailable and can be dosed orally.

### VII. COMBINATION THERAPY

Compositions of the invention are also used in combination with other active ingredients. Preferably, the other active therapeutic ingredients or agents are interferons, monoclonal antibodies, 3CL protease inhibitors, corticosteroids, RNA-dependent RNA polymerase inhibitors, selective serotonin reuptake inhibitors (SSRIs), antihyperuricemic agents, JAK inhibitors, non-nucleoside inhibitors of SARS-CoV-2 and other drugs for treating SARS-CoV-2.

Combinations of the compounds of Formula I-III are typically selected based on the condition to be treated, cross-reactivities of ingredients and pharmaco-properties of the combination. For example, when treating an infection (e.g., SARS-CoV-2), the compositions of the invention are combined with other active therapeutic agents (such as those described herein).

Suitable active therapeutic agents or ingredients which can be combined with the compounds of Formula I-III can include interferons, e.g., pegylated rIFN-alpha 2b, pegylated rIFN-alpha 2a, rIFN-alpha 2a, IFN alpha-2b XL, rIFN-alpha 2a, consensus IFN alpha, corticosteroids, e.g., dexamethasone; monoclonal antibodies, e.g., REGN-COV2, LY-CoV555; SSRIs, e.g., fluoxetine; 3CL protease inhibitors, e.g., PF-00835231, GC376; IL-6 inhibitors, e.g., tocilizumab; anti-inflammatory agents, e.g., amodiaquine; RdRp inhibitors, e.g.; molnupiravir, remdesivir; JAK inhibitors, e.g., baracitinib; antihyperuricemic agents, e.g., probenecid.

In yet another embodiment, the present application discloses pharmaceutical compositions comprising a compound of the present invention, or a pharmaceutically acceptable salt, solvate, and/or ester thereof, in combination with at least one additional therapeutic agent, and a pharmaceutically acceptable carrier or excipient.

According to the present invention, the therapeutic agent used in combination with the compound of this present invention can be any agent having therapeutic effect when used in combination with the compound of the present invention. For example, the therapeutic agent used in combination with the compound of the present invention can be monoclonal antibodies, interferons, 3CL protease inhibitors, RdRp inhibitors, SSRIs, anti-inflammatory agents, antihyperuricemic agents, JAK inhibitors, non-nucleoside inhibitors of SARS-CoV-2, and other drugs for treating SARS-CoV-2.

In another embodiment, the present application provides pharmaceutical compositions comprising a compound of the present invention, or a pharmaceutically acceptable salt, solvate, and/or ester thereof in combination with at least one additional therapeutic agent selected from the group consisting of pegylated rIFN-alpha 2b, pegylated rIFN-alpha 2a, rIFN-alpha 2a, IFN alpha-2b XL, rIFN-alpha 2a, consensus IFN alpha, corticosteroids, e.g., dexamethasone; monoclonal antibodies, e.g., REGN-COV2, LY-CoV555; SSRIs, e.g., fluoxetine; 3CL protease inhibitors, e.g., PF-00835231, GC376; IL-6 inhibitors, e.g., tocilizumab; anti-inflammatory agents, e.g., amodiaquine; RdRp inhibitors, e.g.; molnupiravir, remdesivir; JAK inhibitors, e.g., baracitinib; antihyperuricemic agents, e.g., probenecid, and a pharmaceutically acceptable carrier or excipient.

In yet another embodiment, the present application provides a combination pharmaceutical agent comprising:

a.) a first pharmaceutical composition comprising a compound of the present invention, or a pharmaceutically acceptable salt, solvate, or ester thereof; and

b.) a second pharmaceutical composition comprising at least one additional therapeutic agent selected from the group consisting of SARS-CoV-2 protease inhibiting compounds, SARS-CoV-2 non-nucleoside inhibitors of RdRp, SARS-CoV-2 nucleoside inhibitors of SARS-CoV-2, SARS-CoV-2 3CL protease inhibitors, monoclonal antibodies against the SARS-CoV-2 spike protein, SSRIs, IL-6 inhibitors, anti-inflammatory agents, antihyperuricemic agents, JAK inhibitors, and other drugs for treating SARS-CoV-2, and combinations thereof.

Combinations of the compounds in Formulas I-III and additional active therapeutic agents may be selected to treat patients infected with SARS-CoV-2 and other conditions such as HCoV-229E, HCoV-OC43, or Ebola virus infections. Accordingly, the compounds of Formulas I-III may be combined with one or more compounds useful in treating Ebola virus (EBOV) virus, for example EBOV monoclonal antibodies, SARS-CoV-2 protease inhibiting compounds, SARS-CoV-2 non-nucleoside inhibitors of RdRp, SARS-CoV-2 nucleoside inhibitors of SARS-CoV-2, SARS-CoV-2 3CL protease inhibitors, monoclonal antibodies against the SARS-CoV-2 spike protein, SSRIs, IL-6 inhibitors, anti-inflammatory agents, antihyperuricemic agents, JAK inhibitors, and other drugs for treating SARS-CoV-2, and combinations thereof.

More specifically, one or more compounds of the present invention may be combined with one or more compounds selected from the group consisting of 1.) monoclonal antibodies, e.g. REGN-COV2, LY-CoV555; 2.) corticosteroids, e.g., dexamethasone; 3.) 3CL protease inhibitors, e.g., PF-00835231, GC376; 4.) nucleoside inhibitors of RdRp, e.g., molnupiravir, remdesivir; 5.) SSRIs, e.g., fluoxetine; 6.) IL-6 inhibitors, e.g., tocilizumab; 7.) anti-inflammatory agents, e.g., amodiaquine; 8.) JAK inhibitors, e.g., baracitinib; 9.) antihyperuricemic agents, e.g., probenecid.

It is also possible to combine any compound of the invention with one or more other active therapeutic agents in a unitary dosage form for simultaneous or sequential administration to a patient. The combination therapy may be administered as a simultaneous or sequential regimen. When administered sequentially, the combination may be administered in two or more administrations.

Co-administration of a compound of the invention with one or more active therapeutic agents generally refers to simultaneous or sequential administration of a compound of the invention and one or more of the other active therapeutic agents, such that therapeutically effective amounts of the compound of the invention and one or more of the other active therapeutic agents are both present in the body of the patient.

Co-administration includes administration of unit dosages of the compounds of the inventio before or after administration of unit dosages of one or more other active therapeutic agents, for example, administration of the compounds of the invention within seconds, minutes, or hours of the administration of one or more other active therapeutic agents. For example, a unit dose of a compound of the invention can be administered first, followed within seconds or minutes by administration of a unit dose of one or more of the other active therapeutic agents. Alternatively, a unit dose of one or more other therapeutic agents can be administered first, followed by administration of a unit dose of a compound of the invention within seconds or minutes. In some cases, it may be desirable to administer a unit dose of a compound of the invention first, followed after a period of hours (e.g., 1-12 hours), by administration of a unit dose of one or more other active therapeutic agents. In other cases, it may be desirable to administer a unit dose of one or more other active therapeutic agents first, followed, after a period of hours (e.g., 1-12 hours) by administration of a unit dose of a compound of the invention.

The combination therapy may provide “synergy” and “synergistic”, i.e. the effect achieved when the active ingredients used together is greater than the sum of the effects that results from using the compounds separately. A synergistic effect may be attained when the active ingredients are: (1) co-formulated and administered or delivered simultaneously in a combined formulation; (2) delivered by alteration or in parallel as separate formulations; or (3) by some other regimen. When delivered in alternation therapy, a synergistic effect may be attained when the compounds are administered or delivered sequentially, e.g., in separate tablets, pills or capsules, or by different injections in separate syringes. In general, during alternation therapy, an effective dosage of each active ingredient is administered sequentially, i.e. serially, whereas in combination therapy, effective dosages of two or more active ingredients are administered together. A synergistic anti-viral effect denotes an antiviral effect which is greater than the predicted purely additive effects of the individual compounds of the combination.

In still yet another embodiment, the present application provides for methods of inhibiting SARS-CoV-2 polymerase in a cell comprising: contacting a cell infected with SARS-CoV-2 with an effective amount of compound of Formula I-III or a pharmaceutically acceptable salt, solvate, and/or ester thereof, whereby SARS-CoV-2 polymerase is inhibited.

In still yet another embodiment, the present application provides for methods of inhibiting SARS-CoV-2 polymerase in a cell, comprising: contacting a cell infected with SARS-CoV-2 with an effective amount of compound of Formula I-III, or a pharmaceutically acceptable salt, solvate, and/or ester thereof, and at least one additional active therapeutic agent whereby SARS-CoV-2 polymerase is inhibited.

In still yet another embodiment, the present application provides for methods of inhibiting SARS-CoV-2 polymerase in a cell comprising: contacting a cell infected with SARS-CoV-2 with an effective amount of compound of Formula I-III or a pharmaceutically acceptable salt, solvate, and/or ester thereof, and at least one additional active therapeutic agent selected from the group consisting of interferons, monoclonal antibodies, corticosteroids, 3CL protease inhibitors, SSRIs, IL-6 inhibitors, JAK inhibitors, antihyperuricemic agents, non-nucleoside inhibitors of SARS-CoV-2, and other drugs for treating SARS-CoV-2.

In still yet another embodiment, the present application provides for the use of a compound of the present invention, or a pharmaceutically acceptable salt, solvate, and/or ester thereof, for the preparation of a medicament for treating a SARS-CoV-2 infection in a patient.

### VIII. METABOLITES OF THE COMPOUNDS OF THE INVENTION

Also falling within the scope of this invention are the in vivo metabolic products of the compounds described herein, to the extent such products are novel and unobvious over the prior art. Such products may result for example from the oxidation, reduction, hydrolysis, amidation, esterification and the like of the administered compound, primarily due to enzymatic processes. Accordingly, the invention includes novel and unobvious compounds produced by a process comprising contacting a compound of this invention with a mammal for a period of time sufficient to yield a metabolic product thereof. Such products are typically identified by preparing a radiolabeled (e.g., ¹⁴C or ³H) compound of the invention, administering it parenterally in a detectable dose (e.g., greater than about 0.5 mg/kg) to an animal such as rat, mouse, guinea pig, monkey, or to human, allowing sufficient time for metabolism to occur (typically about 30 seconds to 30 hours) and isolating its conversion products from the urine, blood, or other biological samples. These products are easily isolated since they are labeled (others are isolated by the use of antibodies capable of binding epitopes surviving in the metabolite). The metabolite structures are determined in conventional fashion, e.g., by MS or NMR analysis. In general, analysis of metabolites is done in the same was as conventional drug metabolism studies well-known to those skilled in the art. The conversion products, so long as they are not otherwise found in vivo, are useful in diagnostic assays for therapeutic dosing of the compounds of the invention even inf they possess no SARS-CoV-2 polymerase inhibitor activity of their own.

Recipes and methods for determining stability of compounds in surrogate gastrointestinal secretions are known. Compounds are defined herein as stable in the gastrointestinal tract where less than about 50 mole percent of the protected groups are deprotected in surrogate intestinal or gastric juice upon incubation for 1 hour at 37C. Simply because the compounds are stable in the gastrointestinal tract does not mean that they cannot be hydrolyzed in vivo The prodrugs of the invention typically will be stable in the digestive system but may be substantially hydrolyzed to the parental drug in the digestive lumen, liver or other metabolic organ, or within cells in general

### IX. EXAMPLES

Certain abbreviations and acronyms are used in describing the experimental details. Although most of these would be understood by one skilled in the art, Table 7 contains a list of many of these abbreviations and acronyms.

TABLE 7
List of abbreviations and acronyms.

| Abbreviation | Meaning |
|---|---|
| Ac | acetyl |
| Ac₂O | acetic anhydride |
| Bn | benzyl |
| DCM | dichloromethane |
| DMAP | 4-dimethylaminopyridine |
| DMF | dimethylformamide |
| DMSO | dimethylsulfoxide |
| EDC | ethyl-3-(3-dimethylaminopropyl)carbodiimide |
| EtOAc | ethyl acetate |
| Fmoc | fluorenylmethoxycarbonyl |
| HCl | hydrochloric acid |
| HPLC | High pressure liquid chromatography |
| LCMS | Liquid chromatography mass spectrometry |
| LRMS | Low resolution mass spectrum |
| MeCN | acetonitrile |
| MeOH | methanol |
| MH⁺ | Mass plus 1 |
| MH⁻ | mass minus 1 |
| MS or ms | mass spectrum |
| m/z or m/e | mass to charge ratio |
| rt or r.t. | room temperature |
| TEA | triethylamine |
| TLC or tlc | thin layer chromatography |
| TFA | trifluoroacetic acid |
| UPLC | Ultra-performance liquid chromatography |
| δ | Parts per million down field from tetramethylsilane |

### A. Preparation of Compounds

### Example 1: ((2R,3S,4R,5R)-5-(4-aminopyrrolo[2,1-f][1,2,4]triazin-7-yl)-5-cyano-3,4-dihydroxytetrahydrofuran-2-yl)methyl D-valinate (Compound 1c)

Compound 1a was commercially available from MedChemExpress and synthesized according to previously published procedures (Siegel et al. J. Med. Chem. 2017).

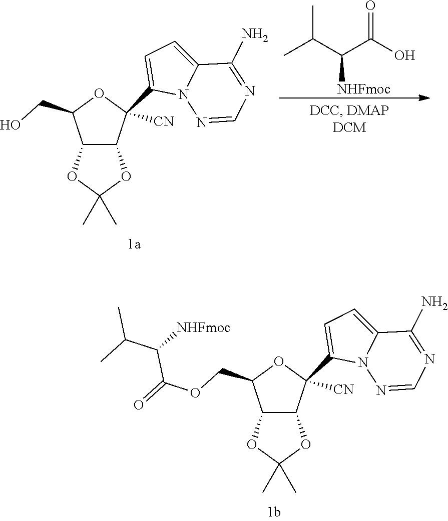

To a solution of (((9H-fluoren-9-yl)methoxy)carbonyl)-L-valine (Fmoc-Val-OH, 7.68 g, 22.6 mmol, 1.50 equiv.) in DCM (150 mL), 1a (5.00 g, 15.0 mmol, 1.0 equiv.), DMAP (2.77 g, 22.6 mmol, 1.5 equiv.), and DCC (4.67 g, 22.6 mmol, 4.58 mL, 1.50 eq) were added sequentially. The reaction was stirred at 25° C. for 1 hour. Then, the crude solution was filtered and the filtrate was isolated and washed sequentially with 15% aq.citric acid (500 mL), brine (500 mL). The crude product was then purified by silica chromatography (SiO₂, DCM/Ethyl acetate=50/1 to 5/1). The purified product 1b was isolated in vacuo as a white solid. Analysis by ESI⁺ (Expected [M+H]⁺=653.71. Observed [M+H]⁺=653.11). ¹H NMR: ET50461-27-P1N1 (400 MHz DMSO-d₆) δ 7.96-7.87 (m, 5H), 7.78-7.69 (m, 3H), 7.42-7.28 (m, 4H), 6.87 (dd, J₁=29.6 Hz, J₂=4.8 Hz, 2H), 5.41 (d, J=6.4 Hz, 1H), 4.92 (dd, J₁=6.4 Hz, J₂=2.8 Hz, 1H), 4.57-4.54 (m, 1H), 4.31-4.16 (m, 5H), 3.91-3.87 (m, 1H), 1.96-1.91 (m. 1H), 1.62 (s, 3H), 1.34 (s, 3H), 0.82 (dd, J₁=17.6 Hz, J₂=6.8 Hz, 2H). ¹³C NMR (125 MHz, CDCl₃) δ 171.59 (s, 1C), 156.13 (s, 1C), 155.21 (s, 1C), 147.48 (s, 1C), 143.69 (s, 2C), 141.26 (s, 2C), 127.69 (s, 2C), 127.05 (s, 2C), 125.03 (d, J=6.16 Hz, 2C), 123.13 (s, 1C), 119.95 (s, 2C), 117.60 (s, 1C), 116.70 (s, 1C), 115.50 (s, 1C), 112.55 (s, 1C), 99.89 (s, 1C), 83.06 (s, 1C), 81.96 (s, 1C), 81.07 (s, 1C), 80.63 (s, 1C), 66.57 (s, 1C), 63.33 (s, 1C), 58.93 (s, 1C), 47.11 (s, 1C), 26.36 (s, 1C), 24.60 (s, 1C), 18.96 (s, 1C), 18.76 (s, 1C).

Five reactions were conducted in parallel.

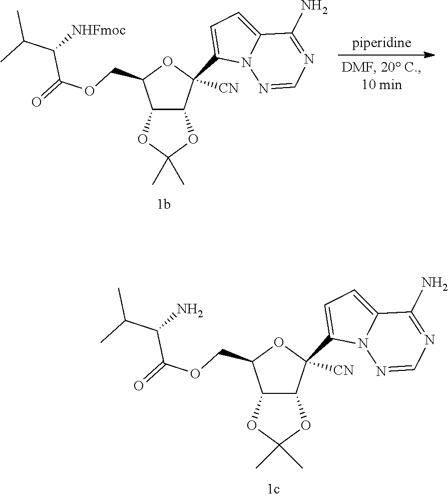

To a solution of 1b (5.00 g, 7.66 mmol, 1.00 eq) in acetonitrile (50.0 mL), piperidine (8.62 g, 101 mmol, 10.0 mL, 13.2 eq) was added. The reaction was allowed to stir at 20° C. for 10 minutes. The mixtures from each of the five reactions were combined and EtOAc (300 mL) was added and the mixture was then washed with twice with brine (300 mL). The organic layer was then dried over Na₂SO₄ and concentrate in vacuo. The crude product was then twice purified by silica chromatography (SiO₂, Dichloromethane/ACN=50/1 to 1/2) and concentrated in vacuo to a white solid. ¹H NMR: (400 MHz DMSO-d₆) δ 7.95 (s, 1H), 6.91 (d, J=4.8 Hz, 1H), 6.83 (d, J=5.2 Hz, 1H), 5.43 (d, J=5.6 Hz, 1H), 4.94 (dd, J₁=6.0 Hz, J₂=2.4 Hz, 1H), 4.59-4.56 (m, 1H), 4.23-4.12 (m, 2H), 2.97 (d, J=5.2 Hz, 1H), 1.74-1.55 (m, 6H), 1.37 (s, 3H), 0.76 (dd, J₁=16.8 Hz, J₂=6.8 Hz, 3H).

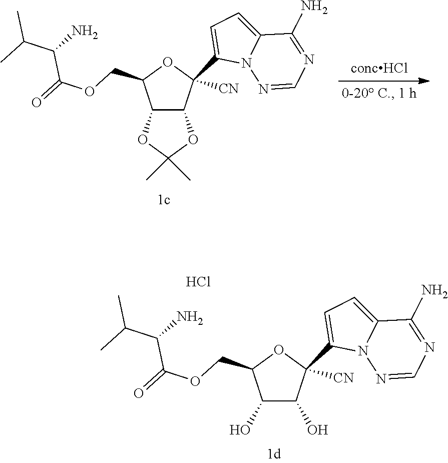 [SMILES: CC(C)[C@H](N)C(=O)OC[C@H]1O[C@@](C#N)(c2ccc3c(N)ncnn23)[C@@H]2OC(C)(C)O[C@@H]21 ; Cl ; CC(C)[C@H](N)C(=O)OC[C@H]1O[C@@](C#N)(c2ccc3c(N)ncnn23)[C@H](O)[C@@H]1O]

Four reactions were conducted in parallel. A solution of 1c (3.00 g, 6.97 mmol, 1.00 eq) in THF (90.0 mL) was cooled to 0° C. Then, 12M HCl (14.5 mL, 25.0 eq) was added, and the solution was allowed to stir at 20° C. for 1 hour. The mixtures from each of the four reactions were then combined and concentrated in vacuo to remove THF. The pH was then adjusted to 2˜3 with NH₃.H₂O and the crude product was purified by preparatory HPLC (Phenomenex luna C18 (250*70mm, 15 um); mobile phase: [water(HCl)-acetonitrile]; B: 0-5%, 20 min). The pH was then adjusted to 4.0 with alkaline resin and dry by lyophilization. 1d was obtained as a white solid (5.80 g, 13.40 mmol, 48.0% yield, 99.3% purity, HCl). Analysis by ESI⁺ (Expected [M+H]⁺=391.40. Observed [M+H]⁺=391.28). ¹H NMR (400 MHz MeOD-d₄) δ 7.90 (s, 1H), 7.01 (d, J=4.8 Hz, 1H), 7.95 (d, J=4.8 Hz, 1H), 4.93 (d, J=5.6 Hz, 1H), 4.65-4.60 (m, 1H), 4.53-4.49 (m, 1H), 4.45-4.41 (m, 1H), 4.13 (dd, J₁=6.4 Hz, J₂=5.2 Hz, 1H), 3.96 (d, J=4.4 Hz, 1H), 2.31-2.23 (m, 1H), 1.03 (dd, J₁=16.4 Hz, J₂=2.4 Hz, 6H). ¹³C NMR (125 MHz, DMSO-d6) δ 175.41 (s, 1C), 156.55 (s, 1C), 147.50 (s, 1C), 123.10 (s, 1C), 116.50 (s, 1C), 116.40 (s, 1C), 109.85 (s, 1C), 100.35 (s, 1C), 80.70 (s, 1C), 78.66 (s, 1C), 73.58 (s, 1C), 69.77 (s, 1C), 62.62 (s, 1C), 59.33 (s, 1C), 31.31 (s, 1C), 18.71 (s, 1C), 16.82 (s, 1C).

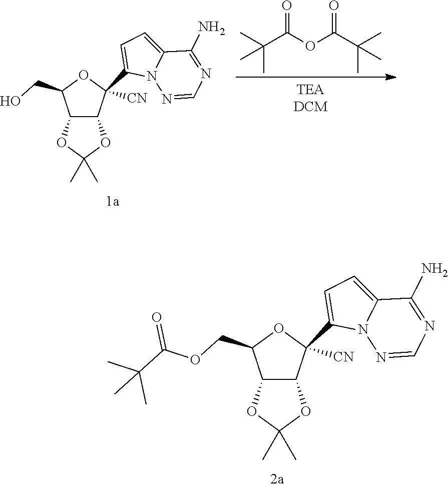

### Example 2: ((3aR,4R,6R,6aR)-6-(4-aminopyrrolo[2,1-f][1,2,4]triazin-7-yl)-6-cyano-2,2-dimethyltetrahydrofuro[3,4-d][1,3]dioxol-4-yl)methyl pivalate (Compound 2b)

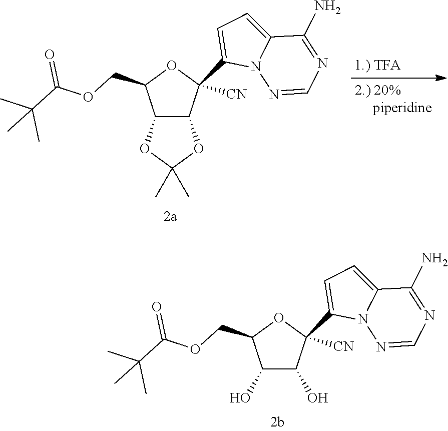 [SMILES: CC1(C)O[C@H]2[C@@H](O1)[C@](C#N)(c1ccc3c(N)ncnn13)O[C@@H]2COC(=O)C(C)(C)C ; CC(C)(C)C(=O)OC[C@H]1O[C@@](C#N)(c2ccc3c(N)ncnn23)[C@H](O)[C@@H]1O]

To a solution of 1a (35 mg, 105.63 μmol) in dichloromethane (1 mL), trimethylacetic anhydride (42.86 μL, 211.27 μmol) and triethylamine (14.33 μL, 211.27 μmol) were added. The crude reaction was allowed to stir at 50° C. for 12 h. Then, the reaction was purified with the sequential addition of the following (1 equivalent each): H₂O, 1M HCl, saturated NaHCO₃, brine, H₂O. The organic layer was then concentrated under reduced pressure and purified by flash chromatography (0-25% Buffer B over 8 minutes. Buffer A: hexanes; Buffer B: EtOAc). Fractions containing the desired compound were combined and concentrated under reduced pressure to afford a colorless oil. Analysis by ESI+. Expected M+H: 416.45. Observed M+H: 416.30. ¹H NMR (300 MHz, CDCl₃) δ 8.18 (s, 1H), 7.33 (d, J=4.87 Hz, 1H), 7.07 (d, J=4.97 Hz, 1H), 5.31 (d, J=6.89 Hz, 1H), 4.86 (m, 1H), 4.62 (m, 1H), 4.39 (dd, J=12.34 Hz 1H), 4.28 (dd, J=12.34 Hz, 1H), 1.79 (s, 3H), 1.39 (s, 3H), 1.35 (s, 3H). ¹³C NMR (75 MHz, CDCl₃) δ 177.76 (s, 1C), 151.21 (s, 1C), 145.49 (s, 1C), 123.11 (s, 1C), 117.42 (s, 1C), 114.96 (s, 1C), 112.78 (s, 1C), 107.98 (s, 1C), 83.93 (s, 1C), 82.67 (s, 1C), 81.62 (s, 1C), 80.88 (s, 1C), 77.01 (s, 1C), 62.07 (s, 1C), 38.60 (s, 1C), 27.13 (s, 3C), 26.20 (s, 1C), 25.28 (s, 1C).

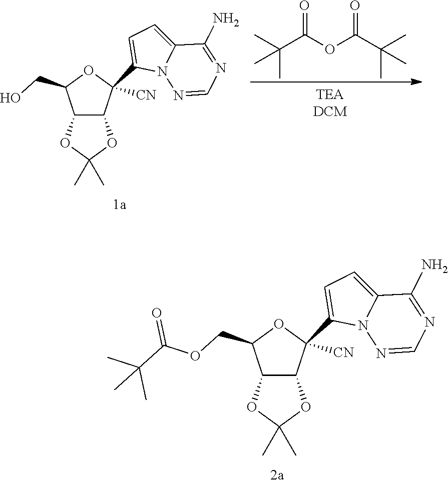

To a solution of 2a in methylene chloride (2 mL), trifluoroacetic acid (50% v/v) was added. The reaction was allowed to proceed with end-over-end rotation at ambient temperature for 12 hours. Then, the reaction was concentrated under reduced pressure and purified by flash chromatography (20-100% Buffer B over 20 minutes; Buffer A: hexanes, Buffer B: EtOAc). The resulting product was then re-dissolved in 20% piperidine in DMF (v/v) and allowed to react with end-over-end rotation for 30 minutes. Then, 5 volumes of DCM were added, and the crude mixture was concentrated under reduced pressure and purified via flash chromatography (20-100% Buffer B over 20 minutes; Buffer A: hexanes, Buffer B: EtOAc). The product was obtained in the acetone fractions, which were combined and concentrated under reduced pressure to a white solid. Analysis by ESI+. Expected M+H: 376.39. Observed M+H: 376.31.

### Example 3: ((3aR,4R,6R,6aR)-6-(4-aminopyrrolo[2,1-f][1,2,4]triazin-7-yl)-6-cyano-2,2-dimethyltetrahydrofuro[3,4-d][1,3]dioxol-4-yOmethyl acetate (Compound 3a)

To a solution of 1a (100 mg, 301.81 μmol) in dichloromethane (2 mL), acetic anhydride (57.06 μL, 603.62 μmol) and triethylamine (81.88 μL, 603.62 μmol) were added. The crude reaction was allowed to stir at 50° C. for 12 h. Then, the reaction was purified with the sequential addition of the following (1 equivalent each): H₂O, 1M HCl, saturated NaHCO₃, brine, H₂O. The organic layer was then concentrated under reduced pressure and purified by flash chromatography (0-25% Buffer B over 8 minutes. Buffer A: hexanes; Buffer B: EtOAc). Fractions containing the desired compound were combined and concentrated under reduced pressure to afford a colorless oil. Analysis by ESI+. Expected M+H: 374.37. Observed M+H: 374.26. ¹H NMR (600 MHz, CDCl₃) δ 8.19 (s, 1H), 7.18 (d, J=4.80 Hz, 1H), 7.18 (d, J=7.46 Hz, 1H), 5.39 (d, J=6.98 Hz, 1H), 4.89 (t, J=5.56, 5.65 Hz, 1H), 4.61 (m, 1H), 4.43 (dd, J=12.42 Hz, 1H), 4.24 (dd, J=12.48 Hz, 1H), 2.60 (s, 3H), 1.74 (s, 3H), 1.39 (s, 3H). ¹³C NMR (125 MHz, CDCl₃) δ 170.50 (s, 1C), 150.94 (s, 1C), 146.11 (s, 1C), 124.39 (s, 1C), 117.28 (s, 1C), 115.18 (s, 1C), 113.74 (s, 1C), 100.51 (s, 1C), 83.85 (s, 1C), 82.92 (s, 1C), 81.95 (s, 1C), 81.20 (s, 1C), 63.28 (s, 1C), 60.37 (s, 1C), 26.31 (s, 1C), 25.01 (s, 1C), 20.71 (s, 1C).

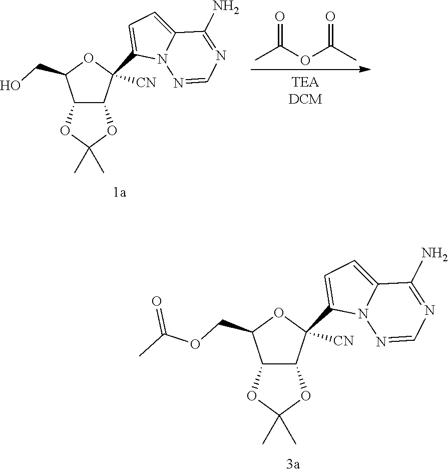

### B. Pharmacological Characterization of Compounds

### Example 4. Bioavailability of CAT002

CAT002 was synthesized and tested as an orally bioavailable prodrug of GS-441524, which is intracellularly converted to GS-443902 an ATP analogue having 3 phosphate groups attached to the 5′ OH of GS-441524. GS-441524 is the core nucleoside of remdesivir. GS-441524 is also the core nucleoside of CAT002. GS-441524 is structurally identical to CAT002 except that GS-441524 does not have the 5′ amino acid ester in CAT002.

CAT002 demonstrated improved water solubility, oral absorption, rapid distribution and elimination, and extensive biotransformation to the core nucleoside, GS-441524, in rats, cynomolgus macaques, and humans. GS-441524 was the predominant species in plasma across all species tested. In rats and cynomolgus macaques, plasma exposures to GS-441524 with respect to Cₘₐₓ were approximately 6-fold higher after oral administration of CAT002 in aqueous solution than those previously reported for equivalent doses of oral GS-441524 in organic solution. In humans, oral administration of CAT002 using a powder-in-capsule formulation resulted in plasma exposures of the core nucleoside approximately 4-fold higher with respect to Cₘₐₓ compared to administration of an oral solution of GS-441524 at an equivalent per-molar dose.

The half-life of GS-441524 derived from CAT002 was 3 hours in rats, 8 hours in cynomolgus macaques, and 7.3 hours in humans; plasma levels of CAT002 were either too low or below the limit of quantification to determine its half-life. Across all species tested, CAT002 was efficiently metabolized to the core nucleoside GS-441524 when administered orally, indicating efficient first-pass intestinal or hepatic metabolism. Rapid hydrolysis of CAT002 in human intestinal homogenates indicated intestinal metabolism as the primary mechanism of CAT002 hydrolysis.

Further in vitro studies indicated the stability of CAT002 and the 87-fold greater aqueous solubility at neutral pH than GS-441524. As summarized in Table 8, these studies indicate that CAT002 is an efficient prodrug of GS-441524 that is well-suited for clinical development as an oral agent.

TABLE 8
In vitro ADME parameters of CAT002 and GS-441524.

| Property | GS-441524 | CAT002 |
|---|---|---|
| Structure | 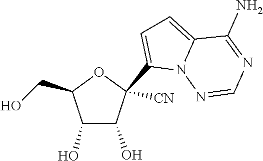 | 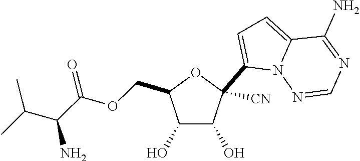 |
| Thermodynamic solubility at pH | 0.04 mg/mL | 3.4 mg/mL |
| 7.4 | | |
| Phosphate buffer stability at | Stable | pH 7.4: 30 hours |
| 25° C., T_{1/2} | | pH 3.0: Stable |
| Caco-2 permeability AB/BA | 0.17/2.1 (Efflux 12.4x) | 0.55/0.45 (Efflux 0.8) |
| (cm/sec•10⁶) | | |
| Human plasma stability at 37° C. | Stable | EDTA plasma: 195 minutes |
| | | Heparin plasma: >289.1 minutes |
| Human intestinal S9 stability at | Stable | 19.5 minutes |
| 37° C. | | |
| Human hepatic microsomal | Stable (+ cofactors) | 67 minutes (− cofactors) |
| stability, T1/2 | | 58 minutes (+ cofactors) |
| (+/− mixed cofactors)* | | |

*NADPH (CYP450) and UDPGA (glucuronidation) cofactors

### 1. Plasma Pharmacokinetics of GS-441524 Following Oral Administration of CAT002 in Sprague-Dawley Rats. In-Life PK

CAT002 (16 mg/kg; 10.8 mg/kg-equivalent of the parent nucleoside, GS-441524) was formulated in saline (1.60 mg/mL) and orally administered to fasted male rats (N=2). Whole blood samples were collected at the following timepoints (hours) in K₂EDTA collection tubes: 0.25, 0.50, 1.00, 2.00, 4.00, 8.00, 24.0, 48.0.

Sample preparation. Plasma was isolated by protein precipitation in a 96-well plate at 0° C. An aliquot of 20 μL unknown sample, calibration standard, quality control and dilution quality control (if have), single blank, and double blank sample was added to the 96-well plate respectively. Each sample (except the double blank) was quenched with 100 μL of IS1 respectively (double blank sample was quenched with 100 μL of MeOH), and then the mixture was vortex-mixed for 10 min at 800 rpm and centrifuged for 15 min at 3220×g, 4° C. An aliquot of 60 μL supernatant was transferred to another clean 96-well plate and centrifuged for 5 min at 3220×g, 4° C., then the supernatant was directly injected for LC-MS/MS analysis.

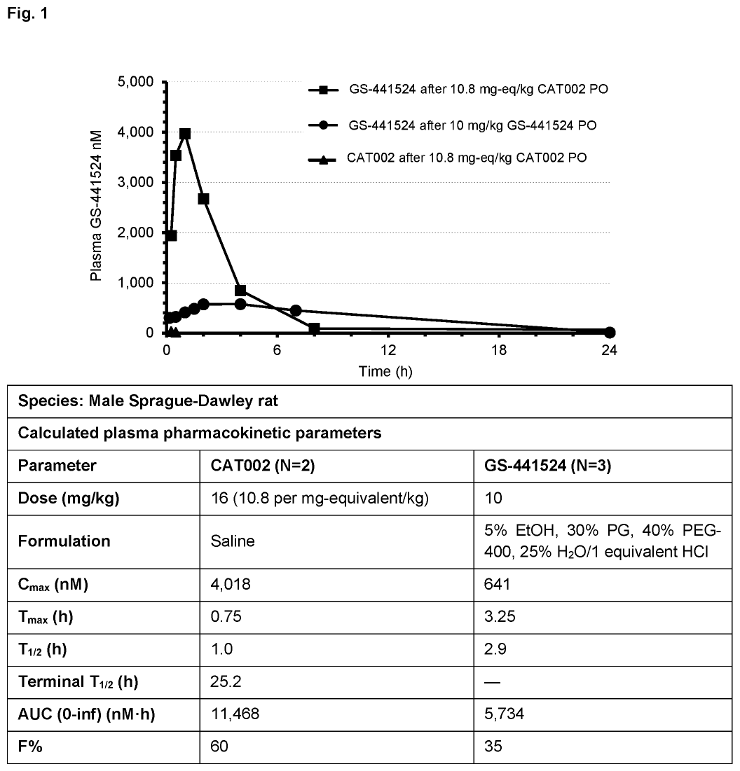

LC-MS/MS analysis. Metabolites of interest (CAT002, GS-441524) were then quantified by LC-MS/MS (SCIEX LC-MS/MS-BZ Triple Quad 6500+, ACQUITY UPLC HSS T3 1.8 μm 2.1×50 mm Column; mobile phase A: 0.1% formic acid in water, mobile phase B: 0.1% formic acid in acetonitrile; flow rate: 0.65 mL/min.). Gradient conditions were as follows: 2% B over 0.2 minutes, 2-25% B from 0.2-1.2 minutes, 25-95% from 1.2-1.4 minutes, 95% from 1.4-2.1 minutes, 95-2% from 2.1-2.11 minutes, 2% from 2.11-2.2 minutes). The lower limit of quantification for both CAT002 and GS-441524 was 2.00 ng/mL. Data for both CAT002 and GS-441524 were fitted and analyzed by Phoenix NonWinLin 6.3 using noncompartmental model 200 (extravascular output). Data on directly administered oral GS-441524 were obtained from the NCATS OpenData Portal and are plotted alongside data generated from CAT002 for comparison (FIG. 1).

### 2. Plasma Pharmacokinetics of GS-441524 Following Oral Administration of CAT002 in Cynomolgus Macaques

CAT002 (7.8 mg/kg; 4.9 mg/kg-equivalent of the parent nucleoside, GS-441524) was formulated in saline (1.60 mg/mL) and orally administered to fasted male cynomolgus macaques (N=2). GS-441524 is in turn intracellularly converted to the bioactive ATP analogue, GS-443902. GS-443902 is known to have an antiviral activity and affinity for viral polymerase. See Gordon et al. JBC 2020; 295(20): 6785-6797; Tchesnokov et al. JBC 2020: 295(47):16156-16165; Yin et al. Science 2020: 368(6498): 1499-1504. Whole blood samples were collected at the following timepoints (hours) in K₂EDTA collection tubes: 0.25, 0.50, 1.00, 2.00, 4.00, 8.00, 24.0, 48.0.

Sample preparation. Plasma was isolated by protein precipitation in a 96-well plate at 0° C. An aliquot of 20 μL unknown sample, calibration standard, quality control and dilution quality control (if have), single blank, and double blank sample was added to the 96-well plate respectively. Each sample (except the double blank) was quenched with 100 μL of IS1 respectively (double blank sample was quenched with 100 μL of MeOH), and then the mixture was vortex-mixed for 10 min at 800 rpm and centrifuged for 15 min at 3220×g, 4° C. An aliquot of 60 μL supernatant was transferred to another clean 96-well plate and centrifuged for 5 min at 3220×g, 4° C., then the supernatant was directly injected for LC-MS/MS analysis.

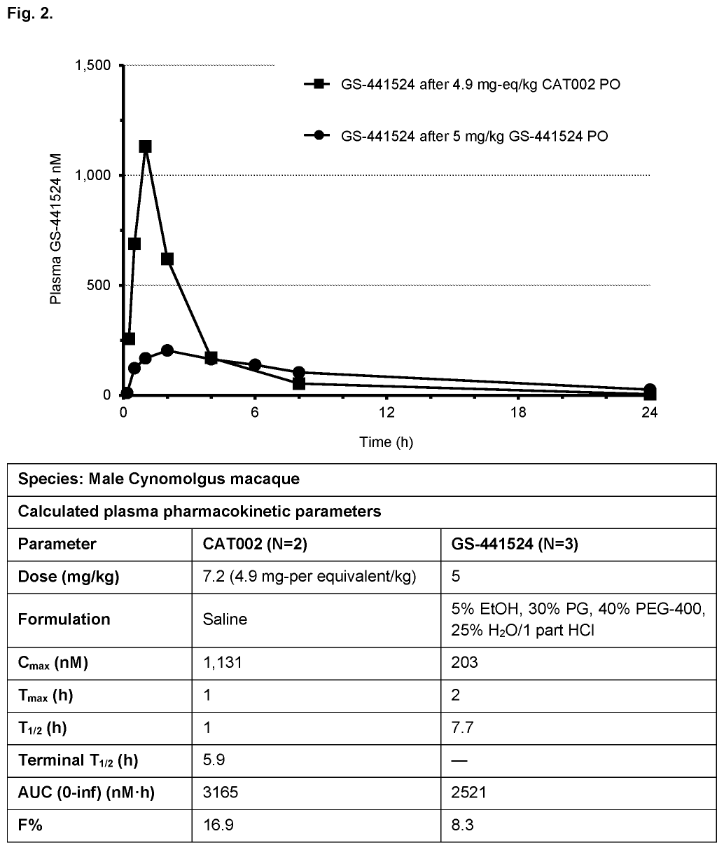

LC-MS/MS analysis. Metabolites of interest (CAT002, GS-441524) were then quantified by LC-MS/MS (SCIEX LC-MS/MS-BZ Triple Quad 6500+, ACQUITY UPLC HSS T3 1.8 μm 2.1×50 mm Column; mobile phase A: 0.1% formic acid in water, mobile phase B: 0.1% formic acid in acetonitrile; flow rate: 0.65 mL/min.). Gradient conditions were as follows: 2% B over 0.2 minutes, 2-25% B from 0.2-1.2 minutes, 25-95% from 1.2-1.4 minutes, 95% from 1.4-2.1 minutes, 95-2% from 2.1-2.11 minutes, 2% from 2.11-2.2 minutes). The lower limit of quantification for 1.00 ng/mL for CAT002 and 2.00 ng/mL for GS-441524. Data for both CAT002 and GS-441524 were fitted and analyzed by Phoenix NonWinLin 6.3 using noncompartmental model 200 (extravascular output). Data on directly administered oral GS-441524 were obtained from the NCATS OpenData Portal and are plotted alongside data generated from CAT002 for comparison (FIG. 2).

### 3. Plasma Pharmacokinetics of GS-441524 Following Oral Administration of CAT002 in Humans. In-Life PK of Orally Administered CAT002

CAT002 (HCl salt) was administered as excipient-less gelatin capsules to healthy participants (N=2) in the fasted state. Participant 1 (Subject 1) is a healthy female and Participant 2 (Subject 2) is a healthy male. Both Participants 1 and 2 were administered 5.4 mg/kg CAT002 (3 mg/kg-equivalent GS-441524). Whole blood samples were collected at the following timepoints (hours) in K₂EDTA collection tubes: 0.5, 1, 3.6, 8, 24, 43. Plasma was isolated by centrifugation and samples were stored at −78° C. before analysis.

In-life PK of orally administered GS-441524. GS-441524 was administered as an excipient-less gelatin capsule (200 mg) to Participant 1 in the fasted state. Whole blood samples were collected at the following timepoints (hours) in K₂EDTA collection tubes: 0.5, 1, 3.6, 8, 24, 43. Plasma was isolated by centrifugation and samples were stored at −78° C. before analysis. Sample preparation. An aliquot of 20 μL unknown sample, calibration standard, quality control and dilution quality control (if have), single blank, and double blank sample was added to the 96-well plate respectively. Each sample (except the double blank) was quenched with 100 μL of IS1 respectively (double blank sample was quenched with 100 μL of MeOH), and then the mixture was vortex-mixed for 10 min at 800 rpm and centrifuged for 15 min at 3220×g, 4° C. An aliquot of 60 μL supernatant was transferred to another clean 96-well plate and centrifuged for 5 min at 3220×g, 4° C., then the supernatant was directly injected for LC-MS/MS analysis.

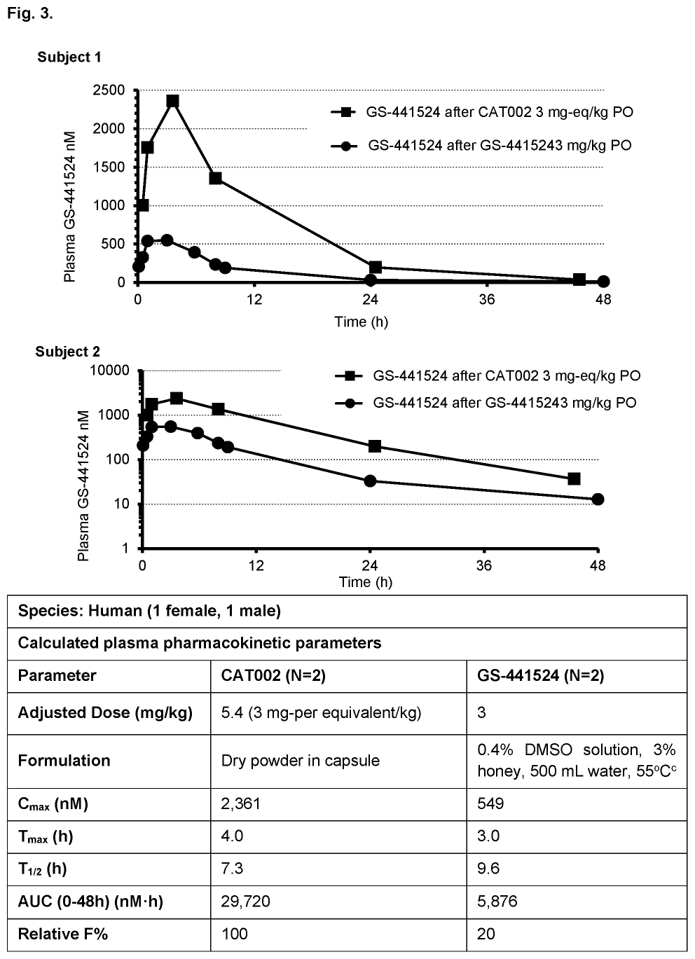

LC-MS/MS analysis. Metabolites of interest (GS-441524) were then quantified by LC-MS/MS (SCIEX LC-MS/MS-BZ Triple Quad 6500+, ACQUITY UPLC HSS T3 1.8 μm 2.1×50 mm Column; mobile phase A: 0.1% formic acid in water, mobile phase B: 0.1% formic acid in acetonitrile; flow rate: 0.65 mL/min.). Gradient conditions were as follows: 2% B over 0.2 minutes, 2-25% B from 0.2-1.2 minutes, 25-95% from 1.2-1.4 minutes, 95% from 1.4-2.1 minutes, 95-2% from 2.1-2.11 minutes, 2% from 2.11-2.2 minutes). The lower limit of quantification was 1.00 ng/mL. Data for both CAT002 and GS-441524 were fitted and analyzed by Phoenix NonWinLin 6.3 using noncompartmental model 200 (extravascular output) (FIG. 3).

All documents, books, manuals, papers, patents, published patent applications, guides, abstracts, and/or other references cited herein are incorporated by reference in their entirety. Other embodiments of the invention will be apparent to those skilled in the art from consideration of the specification and practice of the invention disclosed herein. It is intended that the specification and examples be considered as exemplary only, with the true scope and spirit of the invention being indicated by the following claims.

What is claimed is:

1. A compound or a pharmaceutically acceptable salt or ester thereof, wherein the compound has the structure of Formula I: 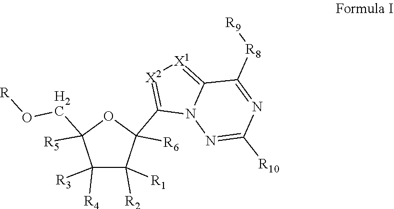
wherein:
R¹, R², R³, R⁴, and R⁵ are each independently H, OR^{a}, N(R^{a})₂, N₃, CN, NO₂, S(O)ₙR^{a}, halogen, (C₁-C₈)alkyl, (C₄-C₈)carbocyclalkyl, (C₁-C₈) substituted alkyl, (C₂-C₈) alkenyl, (C₂-C₈)substituted alkenyl, (C₂-C₈)alkynyl, (C₂-C₈)substituted alkynyl or aryl(C₂-C₈)alkyl; or any two R¹, R², R³, R⁴, or R⁵ on adjacent carbon atoms when taken together are —O(CO)O— or when taken together with the ring carbon atoms to which they are attached form a double bond;
R⁶ is OR^{a}, N(R^{a})₂, N₃, CN, NO₂, S(O)ₙR^{a}, —C(═O)OR¹¹, —C(═O)NR¹¹R¹², —C(═O)SR¹¹, ═S(O)R¹¹, ═S(O)₂R¹¹, —S(O)(OR¹¹), —S(O)₂(OR¹¹), —SO₂NR¹¹R¹², halogen, (C₁-C₈)alkyl, (C₁-C₈)carbocyclylalkyl, (C₁-C₈)substituted alkyl, (C₂-C₈)alkenyl, (C₂-C₈)substituted alkenyl, (C₂-C₈)alkynyl, (C₂-C₈)substituted alkynyl, or (C₆-C₂₀)aryl(C₁-C₈)alkyl;
R⁷ and R⁹ are each independently selected from a group consisting of:
a) H, —C(═O)R¹¹, —C(═O)NR¹¹R¹², C(═O)SR¹¹, ═S(O)R¹¹, ═S(O)₂R¹¹, —S(O)(OR¹¹), —S(O)₂(OR¹¹), —SO₂NR¹¹R¹²; and
b) 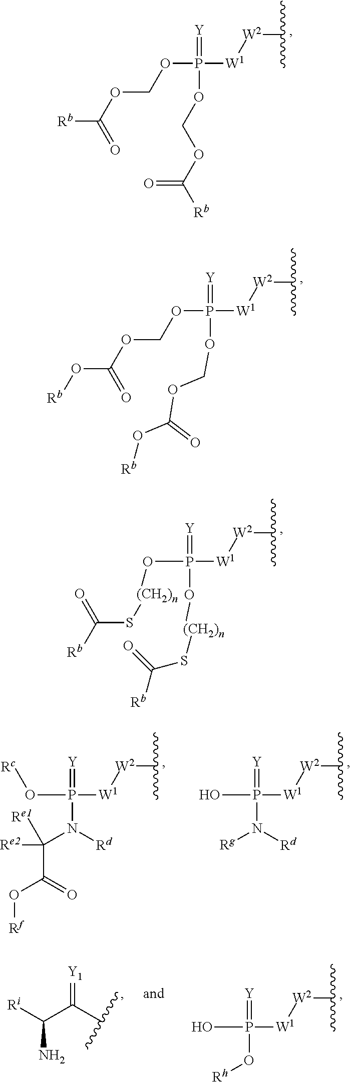
wherein:
Y is O, S, NR, ⁺N(O)(R), N(OR), ⁺N(O)(OR), or N—NR₂;
Y¹ is O or S;
W¹ and W² are each independently O, S, NR, N(OR), CR₂, or C(X⁴)₂;
R^{b} is (C₁-C₈)alkyl, (C₁-C₈)carbocyclylalkyl, (C₁-C₈)substituted alkyl, or (C₆-C₂₀)aryl(C₁-C₈)alkyl;
R^{c} is phenyl, 1-naphthyl, 2-naphthyl, 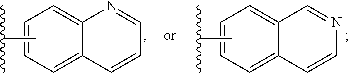
R^{d} is H or CH₃;
R^{e1} and R^{e2} are each independently H, (C₁-C₆)alkyl, benzyl, or halogen;
R^{f} is H, (C₁-C₈)alkyl, benzyl, (C₃-C₆)cycloalkyl, or —CH₂—(C₃-C₆)cycloalkyl;
R^{g} is H, CH₃, (C₁-C₁₂)alkyl,(C₁-C₈)carbocyclylalkyl, (C₁-C₈)substituted alkyl or (C₆-C₂₀)aryl(C₁-C₈)alkyl; alkyl;
R^{h} is H or 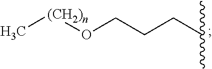
Rⁱ is (C₁-C₈)alkyl, (C₁-C₈)substituted alkyl, (C₁-C₈)carbocyclylalkyl, or (C₆-C₂₀)aryl(C₁-C₈)alkyl;
R⁸ is NH, or NR;
R¹⁰ is H, halogen, NR¹¹R¹², N(R¹¹)OR¹¹, N R¹¹N R¹¹1R¹², N₃, NO, NO₂, CHO, CN, —CH(═NR¹¹), —CH═NHNR¹¹, —CH═N(OR¹¹), —CH(OR¹¹)₂, —C(═O)NR¹¹R¹², —C(═S)NR¹¹R¹², —C(═OR)OR¹¹, R¹¹, OR¹¹, or SR¹¹;
R^{a} is H, (C₁-C₈)alkyl, (C₂-C₈) alkenyl, (C₂-C₈)alkynyl, aryl(C₁-C₈)alkyl, (C₄-C₈)carbocyclylalkyl, (C₁-C₈)substituted alkyl, (C₆-C₂₀)aryl(C₁-C₈)alkyl; alkyl, —C(═O)R¹¹, —C(═O)OR¹¹, —C(═O)NR¹¹R¹², —C(═O)SR¹¹, —S(O)R¹¹, —S(O)₂R¹¹, —S(O)(OR¹¹), —S(O)₂(OR¹¹), or SO₂NR¹¹R¹²;
R¹¹ and R¹² are each independently H, (C₁-C₈)alkyl, (C₂-C₈)alkenyl, or (C₁-C₈)carbocyclylalkyl;
n is 0, 1, or 2;
X¹ and X² are each independently C, C—R¹³, or N; and
R¹³ is H or halogen.

2. A compound or a pharmaceutically acceptable salt or ester thereof, wherein the compound has the structure of Formula II: 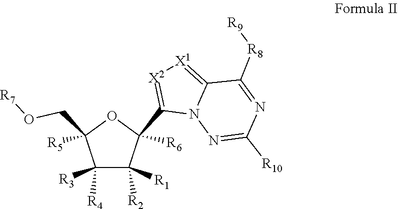
wherein:
R¹, R², R³, R⁴, and R⁵ are each independently H, OR^{a}, N(R^{a})₂, N₃, CN, NO₂, S(O)ₙR^{a}, halogen, (C₁-C₈)alkyl, (C₄-C₈)carbocyclalkyl, (C₁-C₈) substituted alkyl, (C₂-C₈) alkenyl, (C₂-C₈)substituted alkenyl, (C₂-C₈)alkynyl, (C₂-C₈)substituted alkynyl or aryl(C₂-C₈)alkyl; or any two R¹, R², R³, R⁴, or R⁵ on adjacent carbon atoms when taken together are —O(CO)O— or when taken together with the ring carbon atoms to which they are attached form a double bond;
R⁶ is OR^{a}, N(R^{a})₂, N₃, CN, NO₂, S(O)ₙR^{a}, —C(═O)OR¹¹, —C(═O)NR¹¹R¹², —C(═O)SR¹¹, ═S(O)R¹¹, ═S(O)₂R¹¹, —S(O)(OR¹¹), —S(O)₂(OR¹¹), —SO₂NR¹¹R¹², halogen, (C₁-C₈)alkyl, (C₁-C₈)carbocyclylalkyl, (C₁-C₈)substituted alkyl, (C₂-C₈)alkenyl, (C₂-C₈)substituted alkenyl, (C₂-C₈)alkynyl, (C₂-C₈)substituted alkynyl, or (C₆-C₂₀)aryl(C₁-C₈)alkyl;
R⁷ and R⁹ are each independently selected from a group consisting of:
a) H, —C(═O)R¹¹, —C(═O)NR¹¹R¹², C(═O)SR¹¹, ═S(O)R¹¹, ═S(O)₂R¹¹, —S(O)(OR¹¹), —S(O)₂(OR¹¹), —SO₂NR¹¹R¹²; and
b) 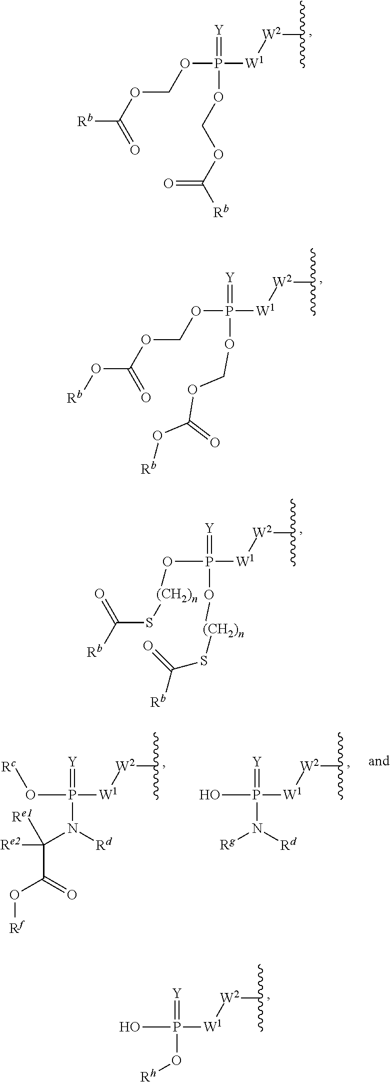
and
c.) 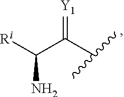
wherein:
Y is O;
Y¹ is O;
W¹ and W² are each independently O, S, NR, N(OR), CR₂, or C(X⁴)₂;
R^{b} is (C₁-C₈)alkyl, (C₁-C₈)carbocyclylalkyl, (C₁-C₈)substituted alkyl, or (C₆-C₂₀)aryl(C₁-C₈)alkyl;
R^{c} is phenyl, 1-naphthyl, 2-naphthyl, 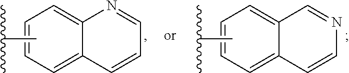
R^{d} is H or CH₃;
R^{e1} and R^{e2} are each independently H, (C₁-C₆)alkyl, benzyl, or halogen;
R^{f} is H, (C₁-C₈)alkyl, benzyl, (C₃-C₆)cycloalkyl, or —CH₂—(C₃-C₆)cycloalkyl;
R^{g} is H, CH₃, (C₁-C₁₂)alkyl,(C₁-C₈)carbocyclylalkyl, (C₁-C₈)substituted alkyl or (C₆-C₂₀)aryl(C₁-C₈)alkyl; alkyl;
R^{h} is H or 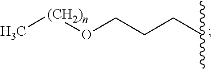
Rⁱ is (C₁-C₈)alkyl, (C₁-C₈)substituted alkyl, (C₁-C₈)carbocyclylalkyl, or (C₆-C₂₀)aryl(C₁-C₈)alkyl;
R⁸ is NH, or NR;
R¹⁰ is H, halogen, NR¹¹R¹², N(R¹¹)OR¹¹, N R¹¹N R¹¹1R¹², N₃, NO, NO₂, CHO, CN, —CH(═NR¹¹), —CH═NHNR¹¹, —CH═N(OR¹¹), —CH(OR¹¹)₂, —C(═O)NR¹¹R¹², —C(═S)NR¹¹R¹², —C(═OR)OR¹¹, R¹¹, OR¹¹, or SR¹¹;
R^{a} is H, (C₁-C₈)alkyl, (C₂-C₈) alkenyl, (C₂-C₈)alkynyl, aryl(C₁-C₈)alkyl, (C₄-C₈)carbocyclylalkyl, (C₁-C₈)substituted alkyl, (C₆-C₂₀)aryl(C₁-C₈)alkyl; alkyl, —C(═O)R¹¹, —C(═O)OR¹¹, —C(═O)NR¹¹R¹², —C(═O)SR¹¹, —S(O)R¹¹, —S(O)₂R¹¹, —S(O)(OR¹¹), —S(O)₂(OR¹¹), or SO₂NR¹¹R¹²;
R¹¹ and R¹² are each independently H, (C₁-C₈)alkyl, (C₂-C₈)alkenyl, or (C₁-C₈)carbocyclylalkyl;
n is 0, 1, or 2;
X¹ is C—R¹³, or N;
X² is C—R¹³; and
R¹³ is H or halogen.

3. The compound or a pharmaceutically acceptable salt or ester thereof according to claim 1, wherein the compound has the structure of Formula III: 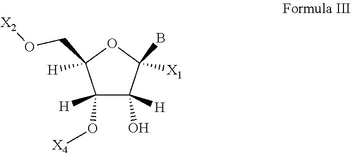
wherein X1 is defined in table 1, X2 is defined in table 2, X4 is defined in table 4, B1 is defined in table 5 and X3 is defined in table 3.

4. The compound or a pharmaceutically acceptable salt or ester thereof according to claim 3, wherein X2 is selected from the group consisting of X2a, X2b, X2c and X2d as defined in table 2.

5. A compound or a pharmaceutically acceptable salt or ester thereof, wherein the compound has the structure of Formula V:

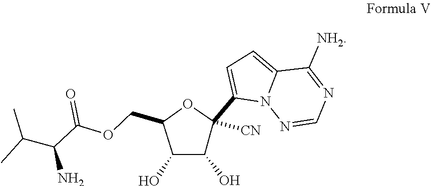

6. The compound or a pharmaceutically acceptable salt or ester thereof according to claim 1, wherein the compound is

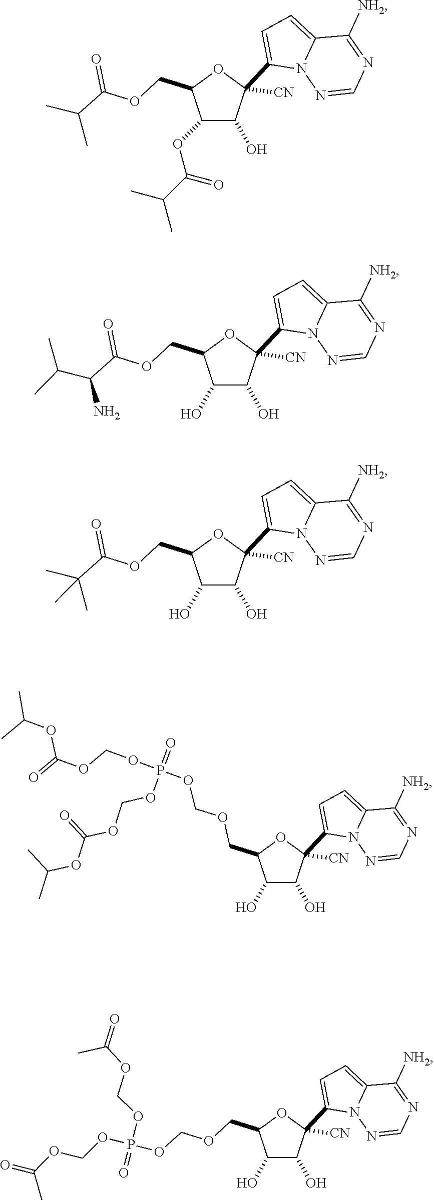

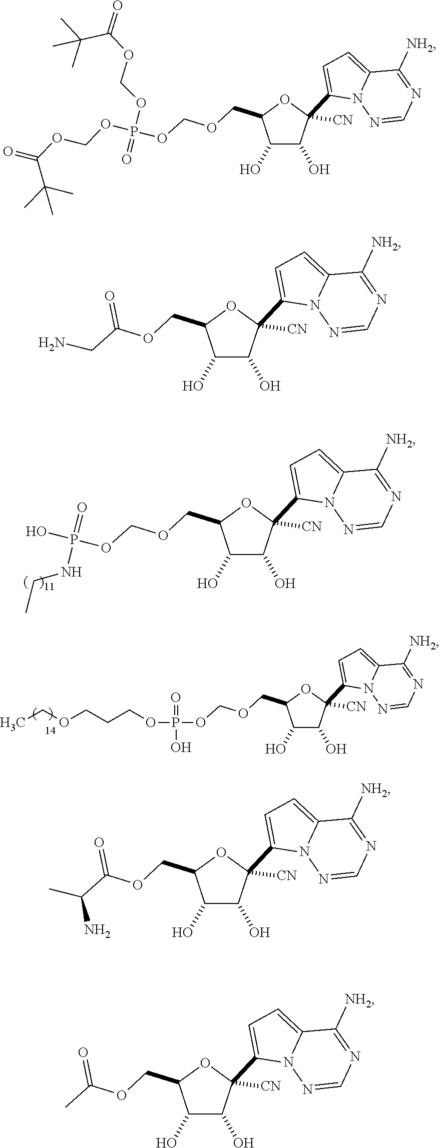

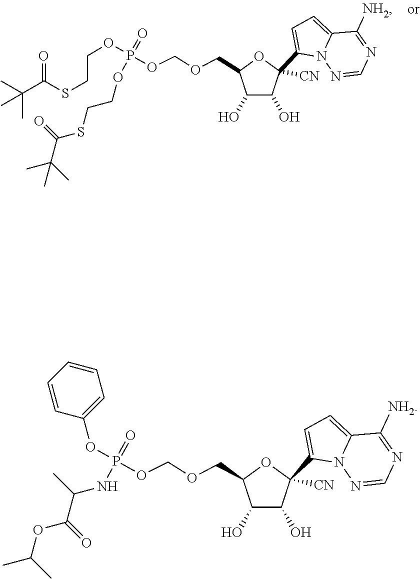

7. A pharmaceutical composition for inhibiting a polymerase of Severe acute respiratory syndrome coronavirus 2 (SARS-CoV-2) (SARS-CoV-2 polymerase), comprising an effective amount of the compound or a pharmaceutically acceptable salt or ester thereof according to claim 5 and a pharmaceutically acceptable carrier.

8. The pharmaceutical composition of claim 7, further comprising an additional therapeutic agent selected from the group consisting of interferons, monoclonal antibodies, corticosteroids, 3CL protease inhibitors, SSRIs, IL-6 inhibitors, JAK inhibitors, antihyperuricemic agents, non-nucleoside inhibitors of SARS-CoV-2, and other drugs for treating SARS-CoV-2.

9. A pharmaceutical composition for treating a viral infection caused by a virus of the Coronaviridae family, comprising an effective amount of the compound or a pharmaceutically acceptable salt or ester thereof according to claim 5 and a pharmaceutically acceptable carrier.

10. The pharmaceutical composition of claim 9, further comprising an additional therapeutic agent selected from the group consisting of interferons, monoclonal antibodies, corticosteroids, 3CL protease inhibitors, SSRIs, IL-6 inhibitors, JAK inhibitors, antihyperuricemic agents, non-nucleoside inhibitors of Severe acute respiratory syndrome coronavirus 2 (SARS-CoV-2), and other drugs for treating SARS-CoV-2.

11. A method of inhibiting a polymerase of Severe acute respiratory syndrome coronavirus 2 (SARS-CoV-2) (SARS-CoV-2 polymerase) in a subject in need thereof, comprising administering to the subject an effective amount of the pharmaceutical composition of claim 7.

12. The method of claim 11, wherein the pharmaceutical composition is administered to the subject orally.

13. The method of claim 11, wherein the effective amount of the pharmaceutical composition is selected to provide a maximum plasma concentration (Cmax) of the compound of Formula V in the subject in the range of 3-10 μM.

14. A method of treating a viral infection in a subject in need thereof, wherein the viral infection is caused by a virus of the Coronaviridae family, comprising administering to the subject an effective amount of the pharmaceutical composition of claim 9.

15. The method of claim 14, wherein the pharmaceutical composition is administered to the subject orally.

16. The method of claim 14, wherein the effective amount of the pharmaceutical composition is selected to provide a maximum plasma concentration (Cmax) of the compound of Formula V in the subject in the range of 3-10 μM.

17. The method of claim 14, wherein the virus is selected from the group consisting of dengue virus, yellow fever virus, West Nile virus, Japanese encephalitis virus, St. Louis encephalitis virus, Omsk hemorrhagic fever, Ebola virus, bovine viral diarrhea virus, Zika virus, Marburg virus, Hepatitis C virus, human coronavirus 229E, human coronavirus OC_{43,} Middle East Respiratory Syndrome virus, Severe Acute Respiratory Syndrome virus, and Severe Acute Respiratory Syndrome coronavirus 2 (SARS-CoV-2).

18. The method of claim 14, further comprising administering to the subject an additional therapeutic agent selected from the group consisting of interferons, monoclonal antibodies, corticosteroids, 3CL protease inhibitors, SSRIs, IL-6 inhibitors, JAK inhibitors, antihyperuricemic agents, non-nucleoside inhibitors of SARS-CoV-2, and other drugs for treating SARS-CoV-2.

19. A method for preparing a composition, comprising mixing the compound or a pharmaceutically acceptable salt or ester thereof according to claim 5 in an effective amount for inhibiting a polymerase of Severe acute respiratory syndrome coronavirus 2 (SARS-CoV-2) (SARS-CoV-2 polymerase) with a pharmaceutically acceptable carrier.

20. A method for preparing a composition, comprising mixing the compound or a pharmaceutically acceptable salt or ester thereof according to claim 5 in an effective amount for treating a viral infection caused by a virus of the Coronaviridae family with a pharmaceutically acceptable carrier.
